(19) United States
(12) Patent Application Publication
(10) Pub. No.: US 2024/0335508 A1
(43) Pub. Date: Oct. 10, 2024

(54) HYBRID GOLD NANOPARTICLE-LIPID NANOPARTICLES AND METHODS OF USE AND PRODUCTION THEREOF

(71) Applicant: THE BOARD OF REGENTS OF THE UNIVERSITY OF OKLAHOMA, Norman, OK (US)

(72) Inventors: Priyabrata MUKHERJEE, Edmond, OK (US); Joshua SEABERG, Oklahoma City, OK (US); Resham BHATTACHARYA, Edmond, OK (US)

(73) Assignee: THE BOARD OF REGENTS OF THE UNIVERSITY OF OKLAHOMA, Norman, OK (US)

(21) Appl. No.: 18/627,146

(22) Filed: Apr. 4, 2024

Related U.S. Application Data
(60) Provisional application No. 63/494,627, filed on Apr. 6, 2023.

(51) Int. Cl. A61K 38/17; A61K 9/107; A61K 9/51; A61K 31/704; A61P 35/00

(52) U.S. Cl. CPC A61K 38/1758; A61K 9/1075; A61K 9/5115; A61K 31/704; A61P 35/00

(57) ABSTRACT

Provided herein are gold nanoparticles (AuNPs) and therapeutics agents co-encapsulated within non-ionic surfactant vehicles (AuNSVs) as well as therapeutic methods of using AuNSVs. Also provided herein are a millifluidic synthesis apparatus and process using ultrasonic mixing for producing AuNSVs encapsulating therapeutic or diagnostic agents, such as chemotherapeutics and/or mRNA.

### REFERENCE TO RELATED APPLICATIONS

The present application claims the priority benefit of U.S. provisional application No. 63/494,627, filed Apr. 6, 2023, the entire contents of which are incorporated herein by reference.

### STATEMENT OF FEDERALLY SPONSORED RESEARCH

This invention was made with government support under grant numbers RO1CA253391-01A1 and 2CA136494 awarded by the National Institutes of Health. The government has certain rights in the invention.

### BACKGROUND

Biomedical nanomaterials display properties distinct from bulk samples of identical materials that offer advantages in therapeutic applications ranging from tissue engineering to drug delivery. While numerous drug and gene delivery systems have been developed as therapeutics, there exists a continual need for increasingly effective nanosystems that can expand the therapeutic envelope in difficult-to-treat conditions. Moreover, a disconnect exists between the laboratory development of novel nanotherapies and the practical implementation of these technologies in clinical care. Lipid-based nanoparticle systems have been shown to effectively overcome numerous hurdles to therapeutic delivery of biologics and small molecule drugs, but challenges in loading, storage stability, and encapsulation efficiency limit the translational potential of these nanotherapeutics. If notable improvements can be made in these areas, lipid-based nanoparticle delivery of therapeutics could be advanced to dramatically alter the paradigm of treatment for numerous conditions.

Lipid-based nanosystems display broad versatility in delivery applications. Liposomes (composed of a lipid bilayer encapsulating an aqueous core) were among the earliest nanosystems and have been utilized to deliver hydrophobic and hydrophilic drugs along with biologics. These systems provide facile surface modification opportunities to promote active targeting and extend circulation time via conjugation with hydrophilic polymers. More recently, researchers have begun development of lipid nanoparticles (LNPs) as delivery vehicles. In contrast to liposomes, LNPs display a primarily hydrophobic lipid core that can retain hydrophobic drugs or nucleic acids through electrostatic complexation between lipids and the encapsulated cargo. These vehicles offer several improvements over liposomes in delivery applications, including improved stability of encapsulated nucleic acids, drug loading, and case of synthesis. A third lipid-based delivery system, the non-ionic surfactant vesicle (niosome), form micelles or lipid bilayers (like liposomes) but are comprised of non-ionic surfactants in place of the phospholipids. These offer notable benefits in material stability and handling but can be limited in delivery capabilities (FIG. 1).

LNPs can display varying internal structures. The first LNPs incorporated a solid lipid core of long-range crystallized lipid structures with emulsifiers and co-surfactants included for structural stabilization; however, the rigidity of the crystal matrix led to reduced drug loading capacity and instability at room temperature. Next generation LNPs, termed nanostructured lipid carriers (NLCs), were developed with varying core structures including imperfect lipid crystal, amorphous, and multiple oil nanodroplet. NLCs generally display increased carrying capacity and improved storage stability over solid LNPs and form the basis of several advanced therapeutic delivery vehicles.

With the development of ionizable lipids, LNPs have become the vehicle of choice for mRNA delivery applications. mRNA provides the information required for ribosomal protein translation, and exogenous mRNA hijacks this cellular machinery to synthesize a specified protein. When utilized medicinally, therapeutic mRNA acts within the cytosol to induce a transient protein expression. Though this strategy has been implemented in COVID-19 vaccines, mRNA technology is not limited to disease prevention. mRNA delivery has been employed in personalized cancer therapy, in wound healing and tissue regeneration, and in treatment of genetic conditions. Nevertheless, rapid mRNA degradation and instability have been a considerable challenge in translating mRNA therapy to the clinic, and optimization of targeted delivery and endosomal escape have yet to be achieved. Improvements in materials for LNP-mediated delivery of mRNA offers potential to overcome these limitations.

LNPs are typically composed of a blend of lipidic components: ionizable lipids complex with mRNA to promote stability and encapsulation, cholesterol and helper lipids form the LNP bulk structure, and PEGylated lipids improve circulation time and pharmacokinetics. Emulsifiers and co-surfactants can be incorporated to maintain structural stability and promote favorable physicochemical properties, and conjugation of targeting moieties to PEG allow for active targeting nanotherapy. The chemical structures and ratios of LNP components affect delivery efficacy; consequently, rigorous effort is expended to develop optimized formulations.

Though heretofore not utilized for mRNA therapy, niosomes (non-ionic surfactant vesicles) have shown promise in delivery of small-molecule therapeutics and offer enhanced stability, biocompatibility, and cost-efficacy in comparison to alternative lipid nanosystems (Kazi et al., J Adv Pharm Technol Res 1, 374-380 (2010)). When modified with cationic lipids, niosomes have been used to deliver macromolecules including antisense oligonucleotides, siRNA, aptamers, and plasmid DNA (Grijalvo et al., Pharmaceutics 11 (2019)). The stability and translational limitations posed by LNP systems could be overcome if a non-ionic surfactant vehicle (NSV) were shown effective for mRNA delivery.

Whereas lipid-based nanosystems are effective for delivery applications, other biomedical nanomaterials display intrinsic properties by which they modulate cellular signaling pathways, catalyze bioreactions, or induce immunological responses. These properties, designated “self-therapeutic” properties, are based purely on material composition, geometry, and functionalization in the absence of alternative therapeutics. Gold nanoparticles (AuNPs) are among the most biologically active self-therapeutic nanomaterials and have been identified to inhibit multicellular crosstalk within the tumor microenvironment, transform cancer-associated fibroblasts to a quiescent phenotype, and inhibit cancer cell growth and migration. Additionally, AuNPs have been shown to sensitize cancer cells to chemotherapeutics and enhance the efficacy of alternative therapeutic strategies. As such, self-therapeutic AuNPs offer significant potential for augmenting and overcoming barriers with existing medical treatments.

In biomedical applications, hybrid nanosystems containing both an inorganic and an organic component outperform single-material analogs. Hybridization between lipid-based materials and inorganic materials offers broad potential for improving delivery characteristics; lipid-based components offer improved solubility, biocompatibility, colloidal stability, and stealth properties while inorganic nanomaterials are included to alter the cellular fusion/uptake interactions, alter carrying capacity, and allow for stimulus-responsivity. siRNA delivered by AuNP-doped liposomes exhibit reduced lysosomal degradation and an altered cellular uptake pathway in comparison to siRNA-loaded control liposomes. MICU1 is a protein that plays a role in cisplatin resistance in ovarian cancer. Dual therapy of MICU1-siRNA-AuroLPs with cisplatin significantly reduced tumor growth in comparison to dual therapy of MICU1-siRNA-cLPs with cisplatin. From this evidence, doping lipid-based nanosystems with AuNPs can greatly improve the delivery efficacy of biologic therapeutics.

Ovarian cancer is the fifth-leading cause of cancer death among women, and we yet lack widely effective therapies to counteract disease progression. High-grade serous ovarian cancer (HGSOC) is the most common type of ovarian cancer, and 95% contain mutations in the tumor suppressor gene TP53. TP53 codes for p53, a protein critical to cancer prevention by regulating the cellular response to stress through activating diverse cellular pathways such as DNA repair, cellular senescence, or apoptosis. Restoration of wild type p53 function has already shown efficacy in treating p53-null prostate cancer, hepatocellular carcinoma (HCC), and non-small cell lung cancer; consequently, delivery of p53 mRNA to TP53-mutant HGSOC with AuNP-doped LNPs could inhibit tumor growth and progression.

### BRIEF DESCRIPTION OF DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIG. 1 shows a comparison and contrast of the features of the main types of lipid-based nanosystems (liposomes, lipid nanoparticles, and niosomes). The non-ionic surfactant vehicles (NSVs) and gold-nanoparticle-doped non-ionic surfactant vehicles (AuNSVs) described herein contain features most associated with niosomes and lipid nanoparticles.

FIGS. 2A-2K show non-ionic surfactant vehicle (NSV) development and characterization. (FIG. 2A) Schematic of AuNSV synthesis process. Lipid components were dissolved in ethanol and aqueous components were dissolved in 25 mM sodium acetate buffer (pH 4). Non-ionic surfactant vehicles were formed by feeding the streams together through a millifluidic tube under sonication. (FIG. 2B) Hydrodynamic diameter of NSVs produced with anionic (stearic acid) or cationic (DDAB) lipid components with varying ratios of TWEEN® 80 (T80). Ratios used (Lipid:cholesterol:T80): 45:50:5; 45:45:10; 40:45:15; 40:40:20; 35:40:25; 35:35:30. (FIG. 2C) Polydispersity index (PDI) of NSVs produced with anionic (stearic acid) or cationic (DDAB) lipid components with varying ratios of TWEEN® 80. (FIG. 2D) Zeta potential of NSVs produced with anionic (stearic acid) or cationic (DDAB) lipid components with varying ratios of TWEEN® 80 measured in 25 mM sodium acetate (pH 4). (FIG. 2E) Hydrodynamic diameter of DDAB NSVs with varying DDAB composition. Ratios used (DDAB:cholesterol:T80): 0:75:25; 10:65:25; 20:55:25; 30:45:25; 40:35:25. (FIG. 2F) PDI of DDAB NSVs with varying DDAB composition. (FIG. 2G) Zeta potential of DDAB NSVs with varying DDAB composition measured in 25 mM sodium acetate (pH 4). (FIG. 2H) Dynamic light scattering (DLS) report of DDAB NSVs with varying DDAB composition. (FIG. 2I) Cell viability of TOV-112D cells exposed to bulk products of NSVs with varying DDAB composition. Viability was assessed using an MTS assay. (FIG. 2J) Hydrodynamic diameter and PDI of NSVs composed with varying lipid components. T80 and cholesterol composition were held constant. (FIG. 2K) Transmission electron micrograph of DDAB NSVs formed with a composition of 35:40:25 DDAB:cholesterol:T80. This ratio was used for all subsequent studies.

FIG. 3 shows additional characterization for NSV development. Composition, size, PDI, and zeta potential of NSVs synthesized with varying ratios of lipid mixture to maleimide-functionalized DSPE-PEG (2 kDa) (wt/wt).

FIGS. 4A-4M show AuNSV characterization and development. (FIG. 4A) Transmission electron micrograph of 20 nm gold nanoparticles (AuNPs). (FIG. 4B) DLS readout of monodisperse 20 nm AuNPs. (FIG. 4C) Zeta potential of 20 nm AuNPs. (FIG. 4D) Diagram showing relative composition of AuNSVs with varying lipid:AuNP ratios. (FIG. 4E) Hydrodynamic diameter of bulk and washed AuNSVs at varying lipid:AuNP ratio. Washing was accomplished using a 30 kDa centrifugal concentrator. (FIG. 4F) PDI of bulk and washed AuNSVs at varying lipid:AuNP ratio. (FIG. 4G) Zeta potential of bulk and washed AuNSVs at varying lipid:AuNP ratio measured in 25 mM sodium acetate (pH 4). (FIG. 4H) Absorbance of bulk and washed AuNSVs (2.5:1 lipid:AuNP) show a distinct AuNP peak over 520 nm. (FIG. 4I) Retention of AuNPs within AuNSVs through the washing process as measured by the ratio of washed to bulk absorbance peaks. (FIG. 4J) Absorbance peak shift when incorporating AuNPs into NSVs. A mixture of AuNPs and NSVs, corresponding to complete AuNP surface association, showed a peak shift of 30 nm. The peak shift for AuNSVs decreased with increasing lipid content, corresponding to enhanced encapsulation of AuNPs. (FIG. 4K) Transmission electron micrograph of AuNSV (2.5:1 lipid:AuNP ratio). (FIG. 4L) Salt stability study of AuNSVs. AuNPs exposed to 750 mM NaCl will aggregate and lose the characteristic absorbance peak, while protected AuNPs do not aggregate and maintain the characteristic absorbance. AuNSVs (2.5:1 lipid:AuNP ratio) show strong protection against AuNP aggregation. (FIG. 4M) Absorbance of AuNSVs loaded with p53-coding mRNA. Around 60% of AuNPs are maintained through the vector washing process.

FIG. 5 shows composition, size, PDI, and zeta potential (measured in sodium acetate buffer pH 4) of AuNSVs (2.5:1 lipid:AuNP wt:wt ratio) synthesized with varying ratios of maleimide-functionalized DSPE-PEG (2 kDa).

FIGS. 6A-6K show storage stability and conditions of AuNSVs. (FIGS. 6A-6F) Characteristic dynamic light scattering plots for NSVs and AuNSVs stored in varying conditions (−20° C. frozen in sodium acetate buffer (25 mM, pH4) or lyophilized and stored at −20° C. with rehydration in either deionized water or normal 0.9% saline). Room temperature samples were measured immediately upon synthesis. Storage of lyophilized samples cryostabilized with 5% sucrose followed with saline rehydration was selected as optimal storage and handling procedure for all AuNSVs used in this study. (FIGS. 6G-6I) Normalized hydrodynamic diameter, PDI, and zeta potential for NSVs and AuNSVs that were stabilized with 5% sucrose, lyophilized, rehydrated in normal saline, and exposed to repetitive freeze/thaw cycles. NSVs and AuNSVs display remarkable retention of physicochemical properties despite exposure to sequential phase changes, allowing for repetitive use of NSV and AuNSV aliquots. (FIG. 6J) Absorbance of AuNSVs through the lyophilization process. While some AuNPs are lost in freeze drying, the majority are retained. (FIG. 6K) Effect of rehydration solution NaCl concentration on measured zeta potential. Rehydrating NSVs and AuNSVs in normal saline (154 mM) decreases the zeta potential of each into the range of positive single digits.

FIGS. 7A-7D show Doxorubicin (DOX)-loaded gold nanoparticle-doped non-ionic surfactant vehicles (AuNSVs). (FIG. 7A) Composition of DOX NSVs and DOX AuNSVs. (FIG. 7B) Absorbance spectra of bulk and washed DOX NSVs and DOX AuNSVs. (FIG. 7C) Encapsulation efficiency of DOX NSVs and DOX AuNSVs. (FIG. 7D) Viability of TOV-112D cells treated with DOX, DOX NSVs, or DOX AuNSVs for 24-, 48-, or 72-hours. DOX AuNSVs showed greater significance in comparison to vehicle controls than either DOX alone or DOX NSVs at low treatment concentrations. Significance at 72-hours was determined using ordinary one-way ANOVA with Tukey's multiple comparisons test. Symbols beside a data point designate a significant difference between that treatment and the matched vehicle control. p<0.05; ** p<0.01; *** p<0.001; **** p<0.0001.

FIG. 8 shows encapsulation and efficacy of doxorubicin (DOX)-loaded AuNSVs. A-C) Size, PDI, and zeta potential of DOX NSVs and DOX AuNSVs.

FIGS. 9A-9H show design and synthesis of mRNA-loaded auro-non-ionic-surfactant vehicles (AuNSVs) for cancer therapy. (FIG. 9A) Schematic illustration of the study. (FIG. 9B) Dynamic light scattering (DLS) readout for p53-mRNA-loaded non-ionic-surfactant vehicles (p53 NSVs) and auro-non-ionic-surfactant vehicles (p53 AuNSVs). For DLS and electrophoretic light scattering (ELS) experiments, nanoparticle samples were measured as diluted 1:100 in rehydration buffer immediately after purification. (FIG. 9C) Mean hydrodynamic diameter of mRNA-loaded lipid nanoparticles and auro-lipid nanoparticles. Each formulation retained a mean hydrodynamic diameter between 125 and 150 nm. (FIG. 9D) Mean polydispersity index (PDI) of mRNA NSVs and AuNSVs. (FIG. 9E) Mean zeta potential for mRNA NSVs and AuNSVs. (FIG. 9F) Encapsulation efficiency of mRNA NSVs and AuNSVs. Co-encapsulation of AuNPs and mRNA did not significantly reduce the carrying capacity of the lipid structure. (FIG. 9G) Absorbance spectrum of p53 AuNSVs displays a peak near 534 nm characteristic of 20 nm gold nanoparticles (AuNPs). (FIG. 9H) Transmission electron micrograph of a p53 AuNSV displays 20 nm AuNPs encapsulated within an amorphous core.

FIGS. 10A-10D show that AuNSVs enhance translation of GFP mRNA in cancer cells. (FIG. 10A) Flow cytometry histograms of GFP expression in TOV-112D cells treated for 24 hours with varying concentrations of GFP mRNA delivered by non-ionic surfactant vehicles (GFP NSVs) or auro-non-ionic surfactant vehicles (GFP AuNSVs). At each concentration, there were greater numbers of cells with higher GFP fluorescence intensity in the groups treated with GFP AuNSVs in comparison to GFP NSVs. (FIG. 10B) Percentage of TOV-112D cells expressing GFP after 24 hours of treatment with varying concentrations of GFP mRNA delivered by GFP NSVs and GFP AuNSVs. The difference in GFP+ cells between treatment groups showed decreasing p-value with increasing concentration. Significance was determined using two-way ANOVA followed by Šídák's multiple comparisons test. (FIG. 10C) Fluorescence microscopy images of GFP delivery in three cancer cell lines (TOV-112D, OVCAR8, and Hep3B) show similar GFP expression profiles when treated with GFP NSVs or GFP AuNSVs at 0.25 μg/mL for 24 hours. (FIG. 10D) Normalized GFP expression in TOV-112D, OVCAR8, and Hep3B cells as measured by image analysis. Five images were taken for each biological replicate. Significance was determined using two-way ANOVA followed by Šídák's multiple comparisons test. n=5 images for TOV-112D; n=15 images for OVCAR8 and Hep3B.

FIGS. 11A-11F shows GFP mRNA delivery using NSVs and AuNSVs. (FIG. 11A) Merged phase contrast and GFP micrographs of GFP-coding mRNA delivery with NSVs or AuNSVs at varying concentrations for 24 hours. (FIG. 11B) Fold increase in proportion of GFP+ cells when GFP mRNA is delivered using AuNSVs vs NSVs. Significance determined using two-way ANOVA variation due to column factor. (FIG. 11C) Merged phase contrast and GFP micrographs of GFP-coding mRNA delivery with NSVs or AuNSVs over time with constant exposure to GFP NSVs or GFP AuNSVs. (FIG. 11D) Quantification of images in FIG. 11C. GFP AuNSV treatment corresponds to greater proportions of GFP+ cells at early time points. Significance determined using two-way ANOVA with Tukey's multiple comparisons test. (FIG. 11E) Merged images of 3-day delivery of Cy5-labeled GFP NSVs and AuNSVs (0.5 μg/mL) to OVCAR8 spheroids. Cy5 GFP AuNSV delivery corresponded to greater Cy5 signal and GFP expression in comparison to Cy5 GFP NSVs. (FIG. 11F) Cy5 and GFP intensity spectra of composite spheroid images (n=3 images). Cy5 GFP AuNSVs show more uniform spheroid distribution in both vector uptake and protein expression.

FIGS. 12A-12N show AuNSV delivery of p53 mRNA activates apoptosis in TP53-null Hep3b cancer cells. (FIG. 12A) Normalized p53 mRNA expression in Hep3B cells after treatment with 1 μg/mL p53 mRNA as delivered by non-ionic surfactant vehicles (p53 NSVs) and auro-non-ionic surfactant vehicles (p53 AuNSVs). p53 AuNSVs delivered twice the quantity of mRNA as a comparable p53 NSV dose. Relative mRNA expression was measured by RT-qPCR. Significance determined using an unpaired two-tailed t-test with Welch's correction. (FIG. 12B) Western blot of p53 in Hep3B cells treated for 24 hours with 1 μg/mL p53 mRNA delivered with p53 NSVs or p53 AuNSVs. (FIG. 12C) Exemplary immunofluorescence microscopy images of Hep3B cells after 24-hour treatment with 1 μg/mL p53 mRNA delivered by p53 NSVs or p53 AuNSVs. (FIG. 12D) Quantification of Hep 3B p53 immunofluorescence images. Five images were taken of each of three biological replicates and significance was determined using an unpaired two-tailed t-test with Welch's correction. (FIG. 12E) Normalized BrdU incorporation in Hep3B cells treated with 1 μg/mL p53 mRNA for 24 hours as measured by ELISA. p53 AuNSV delivery resulted in significantly reduced cell proliferation in comparison to p53 NSV delivery. Empty NSVs and AuNSVs served as vehicle controls while cisplatin (10 μM) served as a positive control. Significance was determined using ordinary one-way ANOVA followed by Tukey's multiple comparisons test. (FIG. 12F) Cell cycle histograms of Hep3B cells treated with 0.25 μg/mL p53 mRNA delivered by p53 NSVs or p53 AuNSVs for 48 hours. p53 AuNSV treatment corresponded to a notable spike in the <G1 population at the expense of the G1 population, suggesting that p53 mRNA therapy acts on cells in the G1 phase to activate apoptosis. (FIG. 12G) Quantification of Hep3B cell populations as determined using a univariate DJF model. NSVs and AuNSVs were utilized as vehicle controls while cisplatin (10 μM) was utilized as a positive control that is known to chelate DNA and retain cells in S phase. (FIG. 12H) Quantification of the <G1 phase cells treated with p53 NSVs and p53 AuNSVs. p53 AuNSV treatment showed significantly increased percentages of <G1 cells, suggesting that p53 AuNSV treatment is more effective at shifting Hep3B cells towards apoptosis than p53 NSV treatment. Significance was determined using an unpaired two-tailed t-test with Welch's correction. (FIG. 12I) Western blot quantification of downstream proteins in the apoptotic and cell-cycle arrest pathways in Hep3B cells treated for 24 hours with 1 μg/mL p53 mRNA delivered with p53 NSVs or p53 AuNSVs. (FIG. 12J) TUNEL apoptosis assay images show greater TUNEL signal in Hep3B cells treated with p53 AuNSVs (24 hours, 1 μg/mL) in comparison to p53 NSVs. (FIG. 12K) Percent of Hep3B cells that are TUNEL positive as quantified by image analysis using ImageJ FIJI. Five images were analyzed for each of three biological replicates. Significance determined using ordinary one-way ANOVA followed by Tukey's multiple comparisons test. (FIG. 12L) Representative colony formation assay for Hep3B cells treated for 10 days with 0.1 μg/mL p53 NSVs or p53 AuNSVs. (FIG. 12M) Histogram of normalized colony area. p53 AuNSV treatment corresponded to fewer and smaller colonies than did p53 NSV treatment. (FIG. 12N) Quantification of Hep3B colony formation assays using image analysis in ImageJ FIJI. p53 AuNSV treatment significantly reduced the clonogenic capacity of Hep3B cells in comparison to both untreated controls and p53 NSV treatment. Three wells were imaged per biological replicate. Significance was determined using ordinary one-way ANOVA followed by Tukey's multiple comparisons test. NT=no treatment.

FIGS. 13A-13L show effects of p53 AuNSV therapy in vitro. (FIG. 13A) Western blot of p53 expression in OVCAR5 cells (1 μg/mL, 24 hours) with treatment of p53 NSVs and p53 AuNSVs. Alpha tubulin was utilized as a loading control. (FIG. 13B) Quantification of p53 expression in OVCAR5 cells. Significance was determined using a two-tailed t-test with Welch's correction. (FIG. 13C) Viability of OV90 cells exposed to a 6-hour pulse of p53 NSVs or p53 AuNSVs with varying concentrations. Viability measured after 24 hours. Significance determined using two-way ANOVA with Šídák's multiple comparisons test. (FIG. 13D) Viability of OVCAR8 cells exposed to a 6-hour pulse of p53 NSVs or p53 AuNSVs (1 μg/mL) with viability measured at 24 and 48 hours. Unloaded NSVs and AuNSVs were used as vehicle controls, while cisplatin (10 μM) was used as a positive control. Significance was determined using two-way ANOVA with Tukey's multiple comparisons test. (FIG. 13E) Caspase 3/7 activation in OVCAR8 cells at the 24-hour time point in experiment (e). Significance determined using one-way ANOVA with Tukey's multiple comparisons test. (FIGS. 13F-13H) Normalized TUNEL signal of OVCAR8, OV90, and Hep3B cells exposed to 1 μg/mL p53 NSVs or p53 AuNSVs. TUNEL area quantified after 24 hours as a ratio to total DAPI area. (FIG. 13I) Colony formation assay for OVCAR8 cells exposed to p53 NSVs or p53 AuNSVs for 10 days. Cells were seeded at a density of 800 per well. (FIG. 13J) OVCAR8 spheroid growth assay. OVCAR8 cells were cultured in non-adherent 96-well plates for 4 days and then exposed to media containing 0.5 μg/mL p53 mRNA delivered with p53 NSVs or p53 AuNSVs. Spheroid exposed to p53 AuNSVs showed delayed growth 48 hours earlier than those exposed to p53 NSVs. Significance determined using two-way ANOVA with Tukey's multiple comparisons test. (FIG. 13K) Viability of OVCAR8 spheroids exposed to p53 NSVs or p53 AuNSVs for 5 days. Significance determined with one-way ANOVA with Tukey's multiple comparisons test. (FIG. 13L) Normalized caspase 3/7 activity in spheroids treated with p53 NSVs or p53 AuNSVs for 5 days. Significance determined with one-way ANOVA with Tukey's multiple comparisons test.

FIGS. 14A-14D show that AuNSVs improve efficacy of p53 mRNA therapy in p53-null Hep3B subcutaneous tumor models. (FIG. 14A) Tumor growth curves throughout the study. Tumor volume was measured with calipers and calculated as V=½lw². (FIG. 14B) Survival curves for tumor growth study. Animals were euthanized on day 26 if they had not reached IACUC-mandated endpoints (tumor volume>1,000 mm³, tumor length>1.5 cm, quality of life). (FIG. 14C) Tumor mass at endpoint. Significance determined using one-way ANOVA with Tukey's multiple comparisons test. (FIG. 14D) Images of tumors collected by treatment group. Scale bar 1 cm.

FIGS. 15A-15E show the effects of p53 AuNSV therapy in vivo. (FIG. 15A) Individual tumor growth curves for the saline control group. (FIG. 15B) Individual tumor growth curves for the AuNSV control group. (FIG. 15C) Individual tumor growth curves for the p53 NSV treatment group. (FIG. 15D) Individual tumor growth curves for the p53 AuNSV treatment group. (FIG. 15E) Tumor volume at the end of treatment for each group. Significance determined using one-way ANOVA with Tukey's multiple comparisons test.

FIGS. 16A-16J show that AuNSVs enhance vector uptake in cancer cells by preferentially activating caveolae-mediated endocytosis. (FIGS. 16A-16C) Flow cytometry histograms showing time-dependent uptake of NBD-labeled non-ionic surfactant vehicles (NBD NSVs) and auro-non-ionic surfactant vehicles (AuNSVs) in TOV-112D cells. Surface NBD fluorescence was quenched using trypan blue to ensure that only endocytosed nanoparticles were included in the analysis. TOV 112D cells showed similar fluorescence intensity between NBD NSV and NBD AuNSV treatment groups at 10 and 30 minutes of uptake, but after 2 hours the cells treated with NBD AuNSVs showed increased uptake in comparison to NBD NSVs. (FIG. 16D) Normalized mean fluorescence intensity (MFI) of TOV-112D cells treated with NBD NSVs or NBD AuNSVs. There was no significant difference in MFI between NBD NSV and NBD AuNSV treatment groups at 10 or 30 minutes of treatment, but there was a significant increase in MFI with NBD AuNSV treatment at 2 hours. Significance determined using two-way ANOVA followed by Šídák's multiple comparisons test. (FIG. 16E) Quantification of TOV-112D uptake inhibition microscopy images for 30-minute treatment with Cy5 NSV and Cy5 AuNSV treatment. Five images were analyzed for each of three biological replicates. Cy5 was utilized in microscopy experiments for reduced autofluorescence. Cy5 signal was normalized to DAPI and compared to that of cells with the same treatment that had no inhibitor. NI=no inhibitor; CPZ=chlorpromazine, clathrin-mediated endocytosis (CME) inhibitor; mβCD=methyl-β-cyclodextrin, caveolin-mediated endocytosis (CvME) inhibitor; filipin, CvME inhibitor; EIPA=5-(N-ethyl-N-isopropyl)-Amiloride, macropinocytosis inhibitor; NaN₃=sodium azide, active transport inhibitor. Significance determined using one-way ANOVA with Dunnett's multiple comparison test. (FIG. 16F) Quantification of TOV-112D uptake inhibition microscopy images for 2-hour treatment with Cy5 NSV and Cy5 AuNSV treatment. Five images were analyzed for each of three biological replicates. (FIG. 16G) Normalized uptake (2-hour delivery) of Cy5 NSVs and Cy5 AuNSVs with no inhibitor, chlorpromazine, or mβCD pretreatment in four cancer cell lines. All four show a preferential activation of CvME for AuNSV uptake. (FIG. 16H) Quantification of fluorescence microscopy images of wild-type (wt) TOV-112D and TOV-112D Cav-1 knockdown (kd) cells treated with Cy5 GFP NSVs and Cy5 GFP AuNSVs for 24 hours. There was a significant increase in Cy5 GFP AuNSV uptake after 24 hours with wt cells, but not in the Cav-1 kd cells. No change was observed with Cy5 GFP NSV treatment. Five images were analyzed for each biological replicate with significance determined using one-way ANOVA with Tukey's multiple comparisons test. (FIG. 16I) Quantification of Cy5 NSV and Cy5 AuNSV uptake in TOV-112D Cav-1 kd cells after 2 hours. Five images were analyzed for each biological replicate with significance determined using an unpaired two-tailed 1-test with Welch's correction. (FIG. 16J) Quantification of TOV-112D Cav-1 kd uptake inhibition microscopy images for 2-hour treatment with Cy5 NSV and Cy5 AuNSV treatment. Five images were analyzed for each of three biological replicates. Significance determined using one-way ANOVA with Dunnett's multiple comparison test.

FIGS. 17A-17J show additional data supporting the AuNSV uptake pathway mechanism. (FIG. 17A) Flow histogram (OVCAR8 cells, 2-hour treatment) showing the effect of trypan blue (TB) surface quenching on nanoparticle uptake quantification. Staining cells with trypan blue causes a left shift in the cell uptake histogram as surface fluorescence is quenched, leaving only internalized fluorescence. (FIG. 17B) Pie charts showing cell populations with internalized, surface adsorbed, or no NBD signal (OVCAR8, 2-hour treatment). (FIG. 17C) Proportion of OVCAR8 cells that are NBD+ for internalized nanoparticles after 2 hours. Significance determined using an unpaired two-tailed t-test with Welch's correction. (FIG. 17D) Normalized mean fluorescence intensity (MFI) of OVCAR8 cells treated with NBD NSVs or NBD AuNSVs for 2 hours. Though the same proportion of cells were NBD+ between treatments, cells treated with NBD AuNSVs showed significantly increased MFI in comparison to NBD NSVs. Significance determined using an unpaired two-tailed t-test with Welch's correction. (FIG. 17E) Uptake inhibition studies in Hep3B cells performed after 2 hours of uptake. Significance calculated using one-way ANOVA with Dunnett's multiple comparisons test. (FIGS. 17F-17G) Characteristic images of Cy5 NSV and Cy5 AuNSV uptake in TOV-112D cells with inhibitor pretreatment. (FIG. 17H) Uptake inhibition studies in OVCAR5 cells performed after 2 hours of uptake. Significance calculated using one-way ANOVA with Dunnett's multiple comparisons test. (FIG. 17I) Uptake inhibition studies in OVCAR8 cells performed after 2 hours of uptake. Significance calculated using one-way ANOVA with Dunnett's multiple comparisons test. (FIG. 17J) Normalized MFI of TOV-112D Cav-1 kd cells treated with NBD NSVs or NBD AuNSVs for 30 minutes. Significance determined using an unpaired, two tailed t-test with Welch's correction.

FIGS. 18A-18J show that AuNSVs promote endosomal escape by inhibiting Rab7-mediated endo-lysosomal fusion. (FIG. 18A) Representative micrographs of TOV-112D cells treated with Cy5-labeled GFP-mRNA-loaded non-ionic surfactant vehicles (Cy5 GFP NSVs) or auro-non-ionic surfactant vehicles (Cy5 GFP AuNSVs) for 24 hours (0.5 μg/mL). Cy5 GFP AuNSVs showed reduced colocalization with Lysotracker™ in comparison to Cy5 GFP NSVs. (FIG. 18B) Normalized Cy5 signal in TOV-112D cells treated with Cy5 GFP NSVs or Cy5 GFP AuNSVs. Increased Cy5 signal corresponds to increased uptake of Cy5 GFP AuNSVs in comparison to Cy5 GFP NSVs. Five images were analyzed for each biological replicate with significance determined using an unpaired two-tailed t-test with Welch's correction. (FIG. 18C) Proportion of Cy5 that colocalized to Lysotracker™ (Manders' colocalization coefficient M1) in TOV-112D cells treated with Cy5 GFP NSVs or Cy5 GFP AuNSVs. Despite increased uptake of Cy5 GFP AuNSVs, fewer of them colocalized to the lysosome in comparison to Cy5 GFP NSVs. Five images were analyzed for each biological replicate with significance determined using an unpaired two-tailed t-test with Welch's correction. (FIG. 18D) Western blot of 20 nm gold nanoparticle (AuNP) and PEGylated 20 nm gold nanoparticle (PEG-AuNP) protein coronas. Both PP2A and Rab7 are enriched in the AuNP protein corona while PEG-AuNPs show reduced binding due to steric inhibition of the AuNP surface. (FIG. 18E) Rab7 activation assay in TOV-112D cells treated with AuNPs and PEG-AuNPs. The GST-RILP fusion protein binds only active Rab7 was immobilized on GSH beads and used to pulldown active Rab7. AuNP treatment reduces the proportion of active Rab7 to total Rab7 while PEGylation of the AuNP surface prevents this from occurring. (FIG. 18F) Quantification of RILP-Rab7 pulldown band intensity with AuNP treatment. Active Rab7 was normalized to total Rab7 for each sample. Significance determined using an unpaired two-tailed t-test with Welch's correction. (FIG. 18G) RILP-Rab7 pulldown assay with NSV and AuNSV treatment. (FIG. 18H) Quantification of RILP-Rab7 pulldown band intensity with NSV and AuNSV treatment. Significance determined using an unpaired two-tailed t-test with Welch's correction. (FIG. 18I) Normalized PP2A activity in TOV-112D cells treated with NSVs or AuNSVs for 24 hours. AuNSV treatment corresponded to a significant reduction in PP2A activity in comparison to NSV treatment. Significance determined using an unpaired two-tailed t-test with Welch's correction. (FIG. 18J) Schematic illustration of the “Trojan horse” mechanism of AuNP-enhanced delivery of mRNA. AuNSVs initially enter the cell non-specifically (CME, macropinocytosis). After an initial endosomal escape, AuNPs within the cytoplasm adsorb and inhibit Rab7 and PP2A, which result in enhanced endosomal escape and a preferential activation of CvME respectively. A flood of additional AuNSVs enter the cell by CvME and escape the trafficking system, allowing ribosomes to bind the delivered mRNA and translate a protein.

FIG. 19 shows a scheme of the design, synthesis, and effects of AuNSV-mediated delivery of therapeutics. (A) Scheme describing the millifluidic process for synthesizing auro-non-ionic surfactant vehicles (AuNSVs). (B) Materials characterization studies undertaken for AuNSV development. (C) In vitro studies characterizing the interactions and effects of AuNSV therapy on cancer cells. (D) In vivo studies confirming p53 AuNSV efficacy as a therapeutic.

FIGS. 20A-20K show additional data supporting the AuNSV trafficking mechanism. (FIGS. 20A-20C) Pearson correlation coefficient, Manders' 2 coefficient, and confirmatory custom-developed % Cy5 on Lysotracker™ calculations for colocalization of Cy5 GFP NSVs and Cy5 GFP AuNSVs with Lysotracker™. TOV-112D cells were treated with 0.5 μg/mL GFP mRNA equivalents for 24 hours before fixation and imaging. Significance determined using unpaired two-tailed t-tests with Welch's correction. (FIG. 20D) DLS chart of AuNPs and PEGylated AuNPs (PEG-AuNPs). PEGylation caused a minor increase in AuNP hydrodynamic diameter. (FIG. 20E) Zeta potential of AuNPs and PEG-AuNPs. Significance determined using an unpaired two-tailed t-test with Welch's correction. (FIGS. 20F-20G) stability of AuNPs and PEG-AuNPs in the presence of 750 mM NaCl. PEGylation protected AuNPs from aggregation. (FIGS. 20H-201) Protein association assays for Rab7 with Hep3B and OVCAR8 cell lysates. (FIG. 20J) PP2A activity assay with 24-hour AuNP and PEG-AuNP treatment. AuNPs significantly reduce the activity of PP2A, and this loss of activity is rescued with AuNP PEGylation. Significance determined using one-way ANOVA with Tukey's multiple comparisons test. (FIG. 20K) Expression of GST-RILP in BL21 cells with 4-hour induction of IPTG at 37° C. in BL21 cells. GST-RILP was isolated using GSH beads.

FIGS. 21A-21J show AuNSV trafficking studies in OVCAR8 cells. (FIG. 21A) Characteristic images of OVCAR8 cells treated with Cy5 GFP NSVs or Cy5 GFP AuNSVs (0.5 μg/mL) for 6- and 24-hours. (FIGS. 21B-21G) Comparisons of Cy5 GFP NSV and Cy5 GFP AuNSV delivery and lysosomal colocalization over time. Significance determined using two-way ANOVA with Šídák's multiple comparisons test. (FIG. 21H) Characteristic images of Cy5 NSV and Cy5 AuNSV trafficking with immunofluorescent staining of Caveolin-1 and Rab7. (FIGS. 21I-21J) Cy5 AuNSVs colocalized to Caveolin-1 and Rab7 to a significantly greater proportion than do Cy5 NSVs as determined by Pearson Correlation Coefficient (PCC). Significance determined using an unpaired two-tailed t-test with Welch's correction.

FIG. 22 shows select lipid components utilized in the present disclosure.

FIG. 23 shows a flow chart for synthesis of AuLNPs in accordance with an embodiment of the present disclosure.

FIG. 24 shows a flow chart for synthesis of AuNSVs in accordance with an embodiment of the present disclosure. Compounds, compositions, quantities, and flow rates shown in the figure are listed only as examples for the purpose of providing context and are not intended to be limiting.

FIG. 25 is a schematic representation of a nanoparticle synthesis apparatus with a membrane filtration system constructed in accordance with the present disclosure.

FIG. 26 is a schematic representation of a nanoparticle synthesis apparatus with a centrifugal concentrator washing system constructed in accordance with the present disclosure.

FIG. 27 shows a syringe pump assembly such as can be used in the nanoparticle synthesis apparatus of FIGS. 25-26.

FIG. 28 shows a reactant feed tube and adapter such as can be used in the nanoparticle synthesis apparatus of FIGS. 25-26.

FIG. 29 shows a tube junction (Y-junction assembly) such as can be used in the nanoparticle synthesis apparatus of FIGS. 25-26.

FIG. 30 shows a nanoparticle synthesis tube such as can be used in the nanoparticle synthesis apparatus of FIGS. 25-26.

FIG. 31 shows an ultrasonic water bath assembly such as can be used in the nanoparticle synthesis apparatus of FIGS. 25-26.

FIG. 32 shows a filtration unit assembly such as can be used in the nanoparticle synthesis apparatus of FIGS. 25-26.

FIG. 33 shows a filtrate tube such as can be used in the nanoparticle synthesis apparatus of FIGS. 25-26.

### DETAILED DESCRIPTION

Delivery of messenger RNA (mRNA) can be utilized to ameliorate numerous pathologies by translation of therapeutic proteins. Researchers have applied this technology throughout the biomedical field, but advances in delivery efficiency and vehicle stability are required to maximize translational potential. Provided herein are gold nanoparticles (AuNPs) and mRNA co-encapsulated within non-ionic surfactant vehicles (AuNSVs) which enhance vector uptake, protein expression, and therapeutic effect in cancer cells as determined using both in vitro and in vivo techniques. This is accomplished through a “Trojan horse” mechanism in which endocytosed AuNPs bind and inactivate PP2A and Rab7, resulting in a preferential shift in uptake toward caveolae-mediated endocytosis and inhibition of endosomal maturation. The present disclosure also describes a millifluidic synthesis apparatus and process for producing non-ionic surfactant vehicles (NSVs) and gold nanoparticle-doped non-ionic surfactant vehicles (AuNSVs) encapsulating a compound for therapeutic or diagnostic delivery. This novel millifluidic NSV synthesis process and apparatus utilizes ultrasonic mixing to induce NSV self-assembly under acoustic cavitation, wherein NSVs are doped with AuNPs to create AuNSVs. NSVs and AuNSVs display low polydispersity, tunable size and zeta potential, and high stability through freeze/thaw phase changes and lyophilization. Within this synthesis process, NSVs and AuNSVs may be loaded with a chemotherapeutic and/or a biologic (i.e. mRNA). In a non-limiting embodiment, doxorubicin (DOX)-loaded AuNSVs display greater differences in cytotoxicity from controls than DOX NSVs or free DOX at low concentrations.

Before further describing various embodiments of the present disclosure in more detail by way of exemplary description, examples, and results, it is to be understood that the compounds, compositions, and methods of present disclosure are not limited in application to the details of specific embodiments and examples as set forth in the following description. The description provided herein is intended for purposes of illustration only and is not intended to be construed in a limiting sense. As such, the language used herein is intended to be given the broadest possible scope and meaning, and the embodiments and examples are meant to be exemplary, not exhaustive. Also, it is to be understood that the phraseology and terminology employed herein is for the purpose of description only and should not be regarded as limiting unless otherwise indicated as so. Moreover, in the following detailed description, numerous specific details are set forth in order to provide a more thorough understanding of the present disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications, and equivalents apparent to those having ordinary skill in the art are included within the scope of the present disclosure. Thus, while the compounds, compositions, and methods of the present disclosure have been described in terms of particular embodiments, it will be apparent to those of skill in the art that variations may be applied to the compounds, compositions, and methods and in the steps or in the sequence of steps of the methods described herein without departing from the concept, spirit, and scope of the inventive concepts.

All patents, published patent applications, and non-patent publications including published articles mentioned in the specification or referenced in any portion of this application are herein expressly incorporated by reference in their entirety to the same extent as if each individual patent or publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular. Where used herein, the specific term “single” is limited to only “one.”

As utilized in accordance with the methods, compounds, and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc. all the way down to the number one (1).

Any numerical range listed or described herein is intended to include, implicitly or explicitly, any number or sub-range within the range, particularly all integers, including the end points, and is to be considered as having been so stated. For example, “a range from 1.0 to 10.0” is to be read as indicating each possible number, including integers and fractions, along the continuum between and including 1.0 and 10.0, that is, having a minimum value equal to or greater than 1.0 and a maximum value equal to or less than 10.0, such as, for example, 3.25 to 8.65. Any maximum numerical limitation recited herein is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein. Thus, even if a particular data point within the range is not explicitly identified or specifically referred to, it is to be understood that any data points within the range are to be considered to have been specified, and that the inventor(s) possessed knowledge of the entire range and the points within the range.

As used in this specification and claims, the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The terms “about” or “approximately,” where used herein when referring to a measurable value such as an amount, a temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment. Further, all references to one or more embodiments or examples are to be construed as non-limiting to the claims.

As used herein, “pure” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

The term “pharmaceutically acceptable” refers to compounds and compositions which are suitable for administration to humans and/or animals without undue adverse side effects such as toxicity, irritation and/or allergic response commensurate with a reasonable benefit/risk ratio. The compounds or conjugates of the present disclosure may be combined with one or more pharmaceutically acceptable excipients, including carriers, vehicles, and diluents which may improve solubility, deliverability, dispersion, stability, and/or conformational integrity of the compounds or conjugates thereof.

The term “active agent” as used herein is intended to refer to a substance which possesses a biological activity relevant to the present disclosure, and particularly refers to therapeutic and diagnostic substances which may be used in methods described in the present disclosure. “Biologically active” refers to the ability of a substance to modify the physiological system of a cell, tissue, or organism without reference to how the substance has its physiological effects.

As used herein any reference to “one embodiment” or “an embodiment” means that a particular element, feature, composition, structure, or characteristic described in connection with the embodiment is included in at least one embodiment. The appearances of the phrase “in one embodiment” in various places in the specification are not necessarily all referring to the same embodiment.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

Where used herein the term “drug combination” refers to a combination of drugs (compounds) which can be conjointly administered.

As used herein the terms “conjoint,” “conjointly,” “conjointly administered,” or “conjoint administration” refers to any form of administration of a combination of two or more different therapeutic compounds (also referred to herein as drugs or active agents) such that the second compound is administered while the previously administered therapeutic compound is still effective in the body, whereby the two or more compounds are simultaneously active in the patient, enabling a synergistic interaction of the compounds. For example, the different therapeutic compounds can be administered either together in the same formulation (i.e., as a physical mixture), or in separate formulations, either concomitantly (at the same time) or sequentially. When administered sequentially the different compounds may be administered immediately in succession, or separated by a suitable duration of time, as long as the active agents function together in a synergistic manner.

The term “effective amount” refers to an amount of an active agent which is sufficient to exhibit a detectable therapeutic effect without excessive adverse side effects (such as toxicity, irritation and allergic response) commensurate with a reasonable benefit/risk ratio when used in the manner disclosed herein. The effective amount for a patient will depend upon the type of patient, the patient's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

The term “ameliorate” means a detectable or measurable improvement in a subject's condition, disease or symptom thereof. A detectable or measurable improvement includes a subjective or objective decrease, reduction, inhibition, suppression, limit or control in the occurrence, frequency, severity, progression, or duration of the condition or disease, or an improvement in a symptom or an underlying cause or a consequence of the disease, or a reversal of the disease. A successful treatment outcome can lead to a “therapeutic effect,” or “benefit” of ameliorating, decreasing, reducing, inhibiting, suppressing, limiting, controlling or preventing the occurrence, frequency, severity, progression, or duration of a disease or condition, or consequences of the disease or condition in a subject.

A decrease or reduction in worsening, such as stabilizing the condition or disease, is also a successful treatment outcome. A therapeutic benefit therefore need not be complete ablation or reversal of the disease or condition, or any one, most or all adverse symptoms, complications, consequences or underlying causes associated with the disease or condition. Thus, a satisfactory endpoint may be achieved when there is an incremental improvement such as a partial decrease, reduction, inhibition, suppression, limit, control, or prevention in the occurrence, frequency, severity, progression, or duration, or inhibition or reversal of the condition or disease (e.g., stabilizing), over a short or long duration of time (hours, days, weeks, months, etc.). Effectiveness of a method or use, such as a treatment that provides a potential therapeutic benefit or improvement of a condition or disease, can be ascertained by various methods and testing assays.

The compounds of the present disclosure may be combined with one or more pharmaceutically acceptable excipients, including carriers, vehicles, diluents, and adjuvants which may improve solubility, deliverability, dispersion, stability, and/or conformational integrity of the compounds or conjugates thereof.

“Pharmaceutically acceptable salts” means salts of active agent compounds disclosed herein which are pharmaceutically acceptable, as defined above, and which possess the desired pharmacological activity. Such salts include (but are not limited to) acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include (but are not limited to) base addition salts which may be formed when acidic protons present are capable of reacting with inorganic or organic bases. Acceptable inorganic bases include (but are not limited to) sodium hydroxide, sodium carbonate, potassium hydroxide, aluminum hydroxide and calcium hydroxide. Acceptable organic bases include (but are not limited to) ethanolamine, diethanolamine, triethanolamine, tromethamine, N-methylglucamine and the like. It should be recognized that the particular anion or cation forming a part of any salt of the present disclosure is not critical, so long as the salt, as a whole, is pharmacologically acceptable. Additional non-limiting examples of pharmaceutically acceptable salts and their methods of preparation and use are shown in Handbook of Pharmaceutical Salts: Properties. and Use (P. H. Stahl & C. G. Wermuth eds., Verlag Helvetica Chimica Acta, 2002).

Where used herein, the terms “sonication” or “sonicating” refer to using sound energy to agitate or deagglomerate nanoparticles in a suspension, generally at rates/frequencies<20 kHz (such as but not limited to 1 Hz-1000 Hz, 10 Hz-500 Hz, 10 Hz-100 Hz, 20 Hz-80 Hz, 25 Hz-75 Hz, 40 Hz-70 Hz, or 50 Hz-60 Hz).

Sonication (or probe sonication) is a process in which sound energy is directly administered to the media/sample by inserting a probe in it. Since the probe is in direct contact with the media/sample, the particles/solvent directly surrounding the probe are hit directly with large amounts of energy causing the formation of bubbles that form and collapse in the surrounding solution, an event known as “cavitation.” These systems require less power input and can deliver up to 20,000 W/L of energy into the processed medium. The properties of the sound energy (power and amplitude) can be controlled.

Where used herein, the terms “ultrasonication” or “ultrasonicating” refer to using rate/frequencies>20 kHz (e.g., in a range of 20 kHz-40 kHz), for homogenization of a fluid. Ultrasonication (or bath sonication), is an indirect sonication method in which a water bath is used. Using this method, the ultrasonic energy is transmitted to a water bath and then into a vessel containing the media/sample. Since the bath sonicator separates/isolates samples from the energy source, it spreads the energy more diffusely throughout the bath and sample. Thus, higher power is required for the process, but the system only delivers 20-40 W/L of energy into the medium.

The term “coadministration” refers to administration of two or more active agents, e.g., a cardiac-targeted composition as described herein and another active agent. The timing of coadministration depends in part on the combination and compositions administered and can include administration at the same time, just prior to, or just after the administration of one or more additional therapies. “Coadministration” is meant to include simultaneous or sequential administration of the compound and/or composition individually or in combination. Thus, the preparations can also be combined, when desired, with other active substances (e.g., to reduce metabolic degradation). For example, the compositions described herein can be used in combination with one another, or with other active agents known to be useful in treating MI, and co-occurring conditions thereof.

The active agents of the present disclosure may be present in the pharmaceutical compositions (alone or in combination) at any concentration that allows the pharmaceutical composition to function in accordance with the present disclosure; for example, but not by way of limitation, the compound(s) may be present in a carrier, diluent, or buffer solution in a wt/wt or vol/vol range having a lower level selected from 0.00001%, 0.0001%, 0.005%, 0.001%, 0.005%, 0.01%, 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9% and 2.0%; and an upper level selected from 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, and 95%. Non-limiting examples of particular wt/wt or vol/vol ranges include a range of from about 0.0001% to about 95%, a range of from about 0.001% to about 75%; a range of from about 0.005% to about 50%; a range of from about 0.01% to about 40%; a range of from about 0.05% to about 35%; a range of from about 0.1% to about 30%; a range of from about 0.1% to about 25%; a range of from about 0.1% to about 20%; a range of from about 1% to about 15%; a range of from about 2% to about 12%; a range of from about 5% to about 10%; and the like. Any other range that includes a lower level selected from the above-listed lower level concentrations and an upper level selected from the above-listed upper level concentrations also falls within the scope of the present disclosure. Percentages used herein may be weight percentages (wt %) or volume percentages (vol %).

The terms long-chain lipid or long-chained lipid refers to a lipid molecule comprising at least one long-chain fatty acid, where long-chain refers to a saturated or unsaturated chain having 13-21 carbon atoms.

Surfactants that may be used in the present disclosure include non-ionic surfactants and ionic surfactants (anionic, cationic, and amphoteric). Examples of non-ionic surfactants which may be used herein include, but are not limited to, Alcohol ethoxylates, Polyethoxylated alcohols, Aliphatic alcohol ethoxylates, Alkyl phenol ethoxylates. Fatty acid etboxylates, Fatty amine ethoxylates, Monoalkanolamide ethoxylates, Sorbitan ester ethoxylates. Ethoxylated fatty alcohols such as BRIJ™-type surfactants (Croda, Wilmington, DE), Ethylene oxide-propylene oxide block copolymers such as PLURONIC™-type and TETRONICT-type copolymers (BASF Corp., Florham Park, NJ), and Alkyl polyglycosides, the following of which are non-limiting examples: Cetomacrogol 1000, Cetostearyl alcohol, Cetyl alcohol, Cocamide DEA, Cocamide MEA, Decyl glucoside, Decyl polyglucose, Glycerol monostearate, IGEPAL® CA-630 (Solvay, Brussels, Belgium), Isoceteth-20, Lauryl Glucoside, Maltoside, Monolaurin, Mycosubtilin, Nonidet P-40™ (Shell Chemical Co., Houston, TX), Nonoxynol-9, Nonoxynols, NP-40, Octacthylene glycol monododecyl ether, N-Octyl beta-D-thioglucopyranoside, Octyl glucoside, Oleyl alcohol, Pentaethylene glycol monododecyl ether, Polidocanol, Poloxamer, Poloxamer 407, Polyethoxylated tallow amine, Polyglycerol polyricinoleate, Polysorbates, Sorbitan, Sorbitan monolaurate, Sorbitan monostearate, Sorbitan tristearate, Stearyl alcohol, Surfactin, Polyoxyethylene sorbitan esters, Polyoxyethylene sorbitan Octoxynol (Triton X-100™, Dow Chemical Co., Midland, MI), Polyoxyl castor oil (CREMOPHOR™, BASF), and Nonylphenol ethoxylate (TERGITOL™, Dow Chemical Co.).

Polysorbate non-ionic surfactants which can be used in the methods of the present disclosure include, but are not limited to, Polysorbate 20 (polyoxyethylene (20) sorbitan monolaurate), a polyoxyethylene sorbitol ester with lauric acid as the primary fatty acid (≥40%) with the balance comprising mainly palmitic, myristic and stearic acids, Polysorbate 40 (polyoxyethylene (20) sorbitan monopalmitate), a polyoxyethylene sorbitol ester with palmitic acid as the primary fatty acid, Polysorbate 60 (polyoxyethylene (20) sorbitan monostearate), a polyoxyethylene sorbitol ester with stearic acid as the primary fatty acid, and Polysorbate 80 (polyoxyethylene (20) sorbitan monooleate), a polyoxyethylene sorbitol ester with oleic acid as the primary fatty acid (≥58%). These are commercially available under the trade names TWEEN® 20, TWEEN® 40, TWEEN® 60, and TWEEN® 80, respectively (Croda).

Examples of ionic surfactants which may be used herein include, but are not limited to, anionic surfactants, cationic surfactants and amphoteric surfactants. Anionic types of surfactants include, for example, Carboxylates, Sulfonates, Petroleum sulfonates, Alkylbenzene sulfonates, Naphthalene sulfonates, Olefin Sulfonates, Sulfates, Alkyl sulfates, Sulfated natural oils and fats, Sulfated esters, Sulfated alkanolamides, and Sulfated alkylphenols. Cationic types of surfactants include, for example, Quaternary ammonium salts, Amines with amide linkages, Polyoxyethylene alkyl amines, Polyoxyethylene alicyclic amines, N,N,N′,N′ Tetrakis substituted ethylenediamines, and Alkyl 1-hydroxyethyl 2-imidazolines. Non-limiting examples of anionic, cationic and amphoteric types of surfactants include the following: Sodium dodecyl sulfate (sodium lauryl sulfate), Sodium laureth sulfate, Lauryl dimethyl amine oxide, Cetyltrimethylammonium bromide (CTAB), Hexadecyltrimethylammonium bromide (HTAB), dodecyltrimethylammonium bromide, Polyoxyl 10, lauryl ether, Bile salts (e.g., sodium deoxycholate, sodium cholate), Methylbenzethonium chloride (HYAMINE™, Lonza Group LLC, Basel, Switzerland), N-Coco 3-aminopropionic acid/sodium salt, N-Tallow 3-Iminodipropionate, disodium salt, N-Carboxymethyl N-dimethyl N-9 octadecenyl ammonium hydroxide, N-Cocoamidethyl N-hydroxyethylglycine, sodium salt, N,N-dimethyldodecylamine-N-oxides, Phosphatidylcholine, and lecithins.

Examples of compounds that can be used as dispersants in the present methods include, but are not limited to, sodium deoxycholate, sodium phosphate, sodium pyrophosphate, potassium pyrophosphate, sodium potassium pyrophosphate, tetrasodium pyrophosphate, tetrapotassium pyrophosphate, potassium citrate buffer, phosphate buffered saline (PBS), sodium bicarbonate, potassium bicarbonate, sodium potassium bicarbonate, sodium carbonate, potassium carbonate, sodium potassium carbonate, amino acids (e.g., glycine, cysteine), sodium phosphate, potassium phosphate, sodium potassium phosphate, sodium acetate, potassium acetate, sodium potassium acetate, tricine, and glycerol.

Where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

Where used herein, the pronoun “we” is intended to refer to all persons involved in a particular aspect of the work disclosed herein and as such may include non-inventor laboratory assistants and collaborators working under the supervision of the inventors.

The following abbreviations may be used herein:
ANOVA: Analysis of Variance,
AuNSV: auro-non-ionic surfactant vehicle,
AuNP: gold nanoparticle,
DAPI: 4,6-diamidino-2-phenylindole,
DDAB: Dimethyldioctadecylammonium bromide,
DLS: Dynamic light scattering,
DMEM: Dulbecco's Modified Eagle medium,
DOPE: 1,2-dioleoyl-sn-glycero-3-phosphoethanolamine,
DOTAP: Dioleoyl-3-trimethylammonium propane,
DOX: Doxorubicin,
DSPE-PEG: 1,2-disteroyl-sn-glycero-3-phosphoethanolamine-N [amino](polyethylene glycol-2000)],
ELS: Electrophoretic light scattering,
EtOH: ethanol,
FBS: fetal bovine serum,
GAPDH: Glyceraldehyde-3-phosphate dehydrogenase,
GFP: Green fluorescent protein,
HGSOC: High-grade serous ovarian cancer,
HRP: horseradish peroxidase,
LNP: lipid nanoparticle,
Mal: Maleimide,
MICU1: Mitochondrial calcium uptake protein 1,
mRNA: messenger RNA,
NBD: nitrobenzoxadiazole,
NLC: nanostructured lipid carrier,
NSV: non-ionic surfactant vehicle
NT: no treatment
PBS: Phosphate-buffered saline,
PDI: Polydispersity Index,
PEG: poly-ethylene glycol,
PFTE: Poly TetraFluoroEthylene
PP2A: protein phosphatase 2A,
RT: room temperature,
SDS: Sodium dodecyl sulfate,
siRNA: small interfering ribonucleic acid,
T80: TWEEN® 80
TBST: Tris Buffered Saline with Tween 20,
TEM: Transmission electron microscopy.

### II. EXAMPLES

Certain embodiments of the present disclosure will now be discussed in terms of several specific, non-limiting, examples. The examples described below will serve to illustrate the general practice of the present disclosure, it being understood that the particulars shown are merely exemplary for purposes of illustrative discussion of particular embodiments of the present disclosure only and are not intended to be limiting of the claims of the present disclosure.

### Example 1

### Methods

### Methods

### Chemicals and Media

Dimethyldioctadecylammonium bromide (98%, DDAB), TWEEN® 80 (viscous liquid, T80), sodium acetate anhydrous, sucrose, stearic acid (175366), stearyl alcohol, hydrochloric acid (HCl), doxorubicin hydrochloride fluorescence grade (DOX), tetrachloroauric acid (HAuCl₄·3H₂O) (520918), sodium citrate tribasic trihydrate (S4641), goat anti-mouse IgG HRP conjugate (H+L, 71045), and rabbit anti-glyceraldehyde-3-phosphate dehydrogenase monoclonal antibody (GAPDH, G9545-100UL) were purchased from Sigma-Aldrich (St. Louis, MO, USA). Dioleoyl-3-trimethylammonium propane (DOTAP), 1,2-dioleoyl-sn-glycero-3-phosphoethanolamine (DOPE), 1,2-dioleoyl-sn-glycero-3-phosphoethanolamine-N-(7-nitro-2-1,3-benzoxadiazol-4-yl) (ammonium salt) (810145, NBD PE), and 1,2-distearoyl-sn-glycero-3-phosphoethanolamine-N-[maleimide (polyethylene glycol)-2000] (ammonium salt) (880126, DSPE-PEG (2000)) were purchased from Avanti Polar Lipids (Alabaster, AL, USA). Cell culture media RPMI 1640 (10-040-CV) and DMEM (10-013-CV) was obtained from Corning Inc. (Corning, NY, USA). FBS (16000-044), Penn-Strep (15140-122), and Goat anti-Mouse IgG (H+L) Secondary Antibody, Alexa Fluor 546 conjugate (A-11030) were purchased from Life Technologies (Grand Island, NY, USA). p53 primary antibody (SC-126, anti-p53 Mouse monoclonal antibody [DO-1]) was purchased from Santa Cruz Biotechnology (Dallas, TX, USA). Anti-alpha-tubulin mouse monoclonal antibody [DM1A] was purchased from Cell Signaling Technology (Danvers, MA, USA). Rabbit IgG HRP conjugated antibody (HAF008) was purchased from Fisher Scientific (Hampton, NH, USA).

### AuNP Synthesis

Colloidal AuNPs (20 nm) were synthesized from a bottom-up process as previously reported (Hossen, M. N. et al. Switching the intracellular pathway and enhancing the therapeutic efficacy of small interfering RNA by auroliposome. Science Advances 6, caba5379, doi: 10.1126/sciadv.aba5379 (2020)). Briefly, a stock solution of 10 mM gold (III) chloride trihydrate was diluted 40× in water and heated up until boiling at 340° C. A prewarmed and filtered solution of 1% sodium citrate tribasic trihydrate was added to the gold chloride solution and allowed to boil for 12-15 min until a red-purple hue was achieved. The AuNPs were removed from the hot plate and stirred overnight on a cool plate. AuNPs were characterized using UV-vis spectroscopy (SPECTROstar Nano®, BMG Labtech), DLS/ELS (Malvern Zetasizer Nano ZS), and TEM (JEOL, Tokyo, Japan).

### Construction of the NSV Synthesis Apparatus

The millifluidic synthesis apparatus was constructed from a NE-1600 syringe pump (New Era Pump Systems, East Farmingdale, NY), an EP30H 280W ultrasonic water bath (Elma, Singen am Hohentwiel, Germany), and PFTE tubing (58700-U, 1/16″ OD×0.031″ ID, Sigma Aldrich, St. Louis, MO). One end of the 260 cm millifluidic synthesis tube was press-fit into the wide end of a 10 μL pipette tip and the narrow end was inserted into the outlet of a polypropylene Nalgene™ ⅛″ ID Y-Type connector (6152-0125PK, Thermo Scientific, Waltham, MS) with a silicone bushing (4 mm×⅛″ OD× 1/16″ ID) placed to ensure liquid seal. Feed tubes were constructed to connect the Y junction to syringes on the syringe pump. Two 10 cm tubes were cut from narrow diameter PFTE tubing (58702, 10 cm× 1/16″×0.012″ ID, Sigma Aldrich, St. Louis, MO), and each tube had a 10 μL tip press fit onto each end. Each of the pipette tips were then trimmed 1 cm from the end, and one end of one tube each was placed in the inlet portions of the Y connector along with a silicone bushing to prevent leaks. Next, the bottom of a 10 cm cell culture dish was removed and the length of the 0.031″ ID tubing was wrapped around the remaining ring and secured in place with parafilm. The millifluidic tube assembly was suspended within the ultrasonic bath beneath a 10 cm by 10 cm foam float held down with a cross support level with the top of the water bath. Approximately 10 cm of tubing was visible above the water at the inlet and 20 cm was visible above the water at the outlet. Bulk LNP products were collected in 15 mL centrifuge tubes suspended within the ultrasonic bath. The system was pressure tested with DI water.

### Lipid Nanoparticle Synthesis

Lipid nanoparticles were synthesized from stock solutions of stearic acid, stearyl alcohol, DDAB, T80, DOPE, DOTAP, and cholesterol. Stearic acid, stearyl alcohol, DDAB, and cholesterol were each dissolved in ethanol (10 mg/mL, 200 proof ethanol) while DOTAP and DOPE were dissolved in tertbutanol to 10 mg/mL and vortexed to mix. Liquid T80 was added to ethanol to achieve a concentration of 10 mg/mL (calculated by density) and vortexed to mix. Varying ratios of lipids were added to a microcentrifuge tube and diluted in ethanol (0.25 mg/mL for low concentration, 5 mg/mL for high concentration). A 1 mL syringe (Beckton Dickinson, 4.78 mm ID) was filled with 500 mL of lipid solution and loaded onto the syringe pump of the millifluidic synthesis apparatus and the plunger was carefully advanced until liquid reached the Y junction.

Next, varying amounts of AuNPs (20 nm) were pelleted from solution (20 minutes at 10k rpm, 8° C.) and the supernatant was removed. The AuNPs were resuspended and added to 4.2 mL of sodium acetate (25 mM, pH 4) in a 10 mL syringe (Beckton Dickinson, 14.5 mm ID) with gentle vortex to mix. The 10 mL syringe was subsequently loaded onto the millifluidic synthesis apparatus. The syringe pump was set to flow at 0.5 mL/min in reference to the smaller syringe, which corresponded to an outlet flow rate of 5.1 mL/min. When the pump and ultrasonic bath were turned on, the lipid and aqueous streams flowed together into the millifluidic tube suspended in the ultrasonic bath to form AuNP-doped NSVs (AuNSVs). At the outlet of the tube, the AuNSVs were collected in a 15 mL centrifuge tube. Newly synthesized AuNSVs were loaded into 30 kDa centrifugal concentrators (Amicon, 30 kDa 0.5 mL; Pall Corp, 30 kDa, 15 mL) and spun for 20-40 minutes (5k rpm for 0.5 mL concentrators, 4k rpm for 15 mL concentrators). Samples were filtered through a 0.2 μm syringe filter and repleted to their original volume in sodium acetate. Samples for lyophilization had sucrose added (50 mg/mL) as a cryoprotectant and were aliquoted into 1.5 mL centrifuge tubes. Tubes were capped with parafilm and punctured to allow sublimation before freezing at −150° C. and lyphilization overnight on a 4.5-L FreeZone benchtop freeze dryer (Labconco, Kansas City, MO). AuNSVs were rehydrated in normal saline with rapid vortexing.

Variations of AuNSVs were synthesized using alterations of the aforementioned synthesis process. NSVs not encapsulating AuNPs were synthesized using sodium acetate alone as the aqueous stream. DiR, Cy5- or NBD-tagged NSVs and AuNSVs were synthesized similarly by incorporating Cy5-PE or NBD-PE into the lipid mixture (25:1 or 1,000:1 lipid:fluorophore-lipid ratio) before loading into the 1 mL syringe. Maleimide-functionalized PEGylated NSVs and AuNSVs were formed by dissolving Mal-DSPE-PEG (2 kDa) in ethanol to 1 mg/mL and mixing at varying ratios with the mixed lipid solution before loading into the syringe. NBD-tagged NSVs and AuNSVs were synthesized similarly by incorporating NBD-PE into the lipid mixture (25:1 or 100:1 lipid:NBD lipid ratio) before loading into the 1 ml syringe. NSVs and AuNSVs encapsulating DOX were synthesized by dissolving DOX in DMSO (10 mg/mL) and adding it to the lipid stream at a 2.5:1 lipid:DOX mass ratio to create DOX NSVs and DOX AuNSVs.

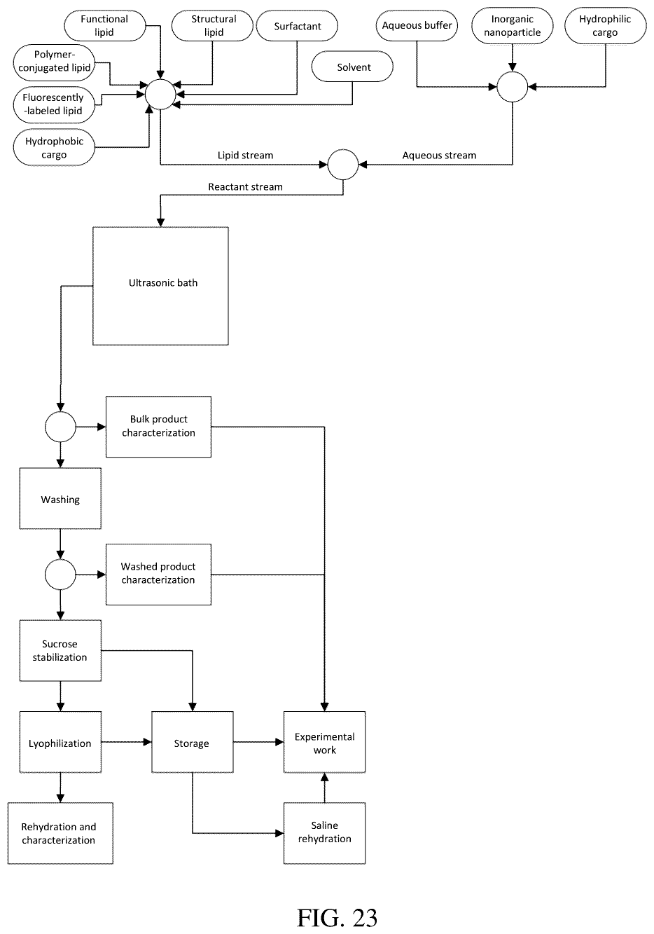

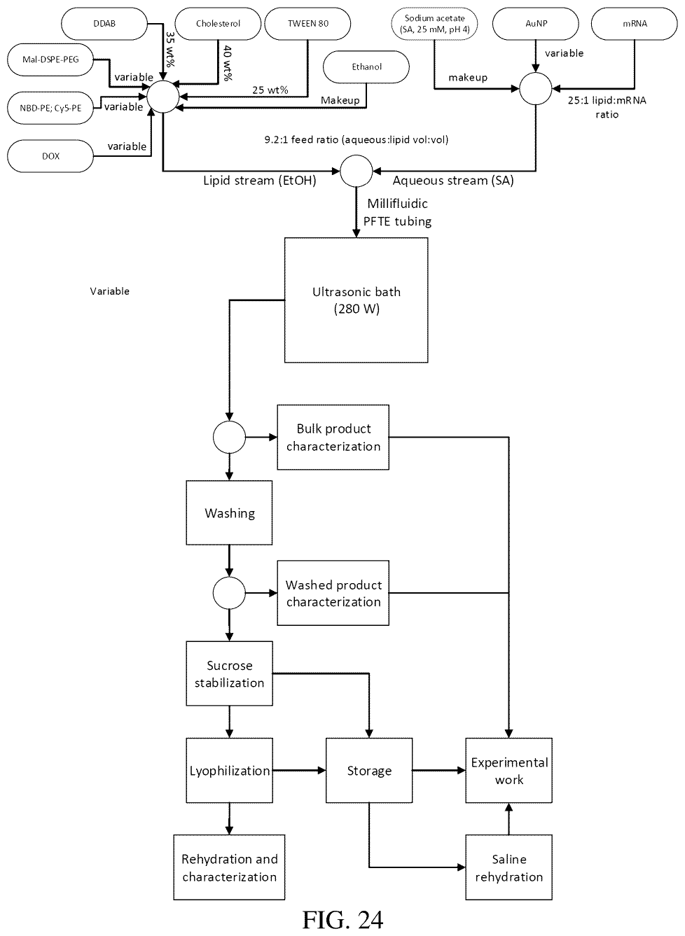

Messenger RNA (mRNA) NSVs and AuNSVs were synthesized using modifications of the millifluidic system as well. The ultrasonic bath was loaded with ice water and all reagents and products were kept on ice throughout the process. BioCap TP53 mRNA (5 mg, PhaRNA LLC, Houston, Texas, USA), BioCap GFP mRNA (5 mg, PhaRNA), or ARCA Cy5 EGFP mRNA (5-moUTP) (10 mg, ApexBio, Houston, TX) was added to the centrifuged AuNPs at a mass ratio of 10:1 AuNP:mRNA and allowed to incubate for 20 minutes at room temperature. After incubation, the AuNPs were resuspended in sodium acetate (25 mM, pH 4) and loaded into the aqueous stream of the millifluidic system. Corresponding mRNA NSVs were created by directly adding an equivalent amount of mRNA to the aqueous stream. A flow diagram for AuNSV synthesis is displayed in FIG. 23.

The volume and concentration of AuNSVs synthesized varied by batch and application. At minimum production, 125 μL of mixed lipids (35:40:25 DDAB:cholesterol:T80) diluted in 125 μL ethanol with a corresponding required volume loaded in the aqueous stream. At maximum production, 500 μL of mixed lipids were diluted in 500 μL ethanol with a corresponding required volume loaded in the aqueous stream.

### Example of a Procedure for Preparing AuLNPs

AuNSV protocol (0.5× vol, 20× conc. Suitable for in vitro and in vivo)
Prepare reagents
   Synthesize 20 nm AuNPs
      Follow published protocols
   Dissolve lipids into 200 proof EtOH
      DDAB (10 mg/mL)
      Cholesterol (10 mg/mL)
      Tween 80 (10 mg/mL by density)
   Prepare sodium acetate buffer
      25 mM, pH 4
Spin down AuNPs
   Add 1.43 mL AuNPs (40 μg/mL) into a microcentrifuge tube
   Spin at 10k rpm for 20 min at 8° C.
   Remove supernatant to AuNP waste-leave 30 μL in the tube
   Resuspend and combine concentrated AuNPs
Dilute and mix lipids
   Create working lipid mixture by adding 400 μL cholesterol, 350 μL DDAB, and 250 μL T80 into a microcentrifuge tube and pipette to mix
   Remove 125 μL lipid mixture and add to a new microcentrifuge tube
   Add 125 μL EtOH onto 125 μL lipid mixture and pipette to mix
   Use 1 mL pipette to remove 250 μL lipid mixture from centrifuge tube and load into 1 mL syringe
      This is now ready to load onto the syringe pump
   Load 1 mL syringe onto syringe pump
      Connect lipid stream tube to syringe tip
   Start the syringe pump (500 μL/min) and watch until liquid reaches Y junction, then stop the syringe pump
Prepare aqueous stream
   Observe the required volume to fill a 10 mL syringe to the same point as the 1 mL syringe is at, to push 250 μL liquid to the Y junction (˜ 2.6 mL)
   Load this volume of sodium acetate into the 10 ml syringe minus AuNP volume
   Add concentrated AuNPs to syringe directly with 1 mL pipette
   Vortex to mix
      If at any point you see the color change from red to blue/purple, throw away the AuNPs and start over
   Remove air bubbles and load syringe onto syringe pump
   Connect aqueous tubing side
Synthesize AuNSVs
   Fill water bath up to the line with fresh water
      If you are making several batches, bring ice to cool down the sonic bath as it will get warm quickly
   Attach a labeled 15 mL centrifuge tube to the side of the water bath with the bottom inside the bath
   Place tube outlet in the top of the centrifuge tube
   Turn on sonic bath
   Start syringe pump at 500 μL/min
   Check all connections to ensure that there are no leaks
   Push until the syringes have been fully cleared.
      You will hear a clicking sound from the syringe pump screw catching and releasing
   Stop syringe pump but leave sonic bath on
   Pull out the tubes connected to the syringes
   Pull the plungers of the syringes back to their initial volume
   Reconnect the tubes to the syringes
   Start the syringe pump again
      Pushes air plug through the system to ensure you get all the material on the other side
   Once air plug reaches the tube outlet, you may turn off the syringe pump and the sonic bath
Purify the nanoparticles
   Take bulk AuNSV sample and remove 500 μL for future characterization
   Place the remaining bulk AuNSV sample in a 30 kDa centrifugal concentrator
      15 mL concentrators: spin at 4000 rpm for 20 min
      500 μL concentrators: spin at 5000 rpm for 20 min 2×
   Resuspend the retentate in the concentrator
   Transfer retentate to centrifuge tube
   Add sodium acetate buffer to return volume to initial bulk sample loaded
   Filter washed product through 0.22 μm syringe filter
Characterize AuLNPs
   Add 1 mL of AuNSVs (diluted 1:100 in saline) to DLS cuvette
   Size with standard protocols
   Add 600 μL from DLS cuvette to ELS cuvette
   Check zeta with standard protocols
   Return samples to washed product collection
   Add 100 μL of bulk AuNSVs, washed AuNSVs, and sodium acetate buffer to 96 well plate (3× for replicates)
   Measure absorbance spectra
   Calculate % AuNP retention by ratio of peaks at 534 nm
Store AuNSVs
   Add sucrose directly to AuNSVs to create 50 mg/mL concentration
   Vortex vigorously to dissolve
   AuLNPs can be frozen and rehydrated in this form >5× with minimal effect on size/PDI
Freeze dry AuNSVs
   Aliquot AuNSVs in 5% sucrose into microcentrifuge tubes (0.5 mL volume)
   Add double layered parafilm covering
   Perforate parafilm with insulin syringe˜20 times
   Freeze at −150 C
   Defrost lyophilizer
   Start collector
   Start vacuum.
      Check to ensure oil valve is open (facing right)
   Place AuNSV samples on lyophilizer overnight
   Collect the next morning, remove parafilm, store at −20 C
   Rehydrate AuNSVs as necessary with normal saline

### Variations of AuNSV Synthesis

Variations in batch size
   For larger batches, increase volume
   Can decrease concentration for optimization
RNA loading
   Incubate RNA with concentrated AuNSVs at RT for 20 mins before adding to aqueous stream
Control NSVs
   Perform same procedure but no AuNPs
Fluorescently tagged NSVs
   Add NBD-PE, Rhd-PE, or Cy5-PE to lipid mixture at either 25:1 or 100:1 wt/wt ratio.
      25:1 good for 1× conc, 1× volume
      100:1 better for 20× conc, 0.5× vol
Functionalized PEG AuNSVs
Add PEGylated lipids at 25:1 or 100:1 ratios

### AuNSV Characterization

AuNSVs were characterized with DLS (Malvern Instruments Zetasizer, Malvern, UK) to determine the hydrodynamic diameter and polydispersity index (PDI). For low concentration samples, 800 μL of sample were added to a transparent cuvette and light scattering was measured at a 90° angle with 3 measurements reported as one replicate. For high concentration samples, after washing, 10 μL were added to 1,000 μL normal saline in a transparent cuvette and light scattering was measured at a 90° angle with 3 measurements reported as one replicate. Data reported is the average and standard deviation of at least 3 independent replicates. This solution was transferred to a zeta cuvette and used to measure the sample zeta potential.

### AuNSV Zeta Potential Measurement

Nanoparticle zeta potential was determined using ELS (Malvern Instruments Zetasizer, Malvern, UK). For low concentration samples, 600 μL of sample were added to a zeta potential cell and measured with at least 3 measurements reported as one replicate. For high concentration samples, 600 μL of the sample used for DLS was transferred to the zeta potential cell and measured directly. Data reported is the average and standard deviation of at least 3 independent replicates.

### AuNSV Absorbance and Encapsulation Efficiency

Doxorubicin and AuNP encapsulation were determined using absorbance spectroscopy. AuNSVs were loaded into a 96-well plate (Thermo Fisher Scientific, Hampton, NH, USA) in 100 μL aliquots. Samples of the unwashed bulk product and the washed product were included to allow quantification of encapsulation by through the ratio of absorbance peaks. DOX encapsulation was measured at 480 nm using a SPECTROstar® plate reader (BMG Labtech, Ortenberg, Germany). AuNP encapsulation was measured at 538 nm using a SPECTROstar plate reader (BMG Labtech, Ortenberg, Germany). mRNA encapsulation was determined using a Quant-it™ RiboGreen RNA Assay Kit (Thermo Fisher Scientific, Hampton, NH). An RNA standard curve was prepared as per the manufacturer's protocol. Washed and bulk samples of mRNA AuNSVs were broken apart in 2% Triton X-100 for 30 minutes at 37° C. before 1× Ribogreen dye was added in a 1:1 volume ratio. Sample fluorescence (ex. 485 nm, em. 538 nm) was read using a SPECTROstar® plate reader to determine values for bulk and processed samples of mRNA NSVs and mRNA AuNSVs. Encapsulation efficiency was calculated from the ratio of mRNA loaded into the system (bulk product) and the mass recovered from the concentration and volume of the purified mRNA AuNSV product (washed product).

### Stability of AuNSVs

NSVs and AuNSVs were synthesized as described above. Samples (100 μL) of AuNSVs with varying lipid:AuNP ratios were loaded into a 96 well plate. Immediately after sample loading, 100 mL of either sodium acetate (25 mM, pH 4) or sodium chloride (1.5 M) was added to the well. The plate was incubated at 37° C. for at least 2 hours before the absorbance spectra of each well was measured with a SPECTROstar® plate reader (BMG Labtech, Ortenberg, Germany). Absorbance peak shift was calculated by subtracting the averages of previously collected AuNSVs wavelength peaks from the AuNP peak (526 nm).

### Electron Microscopy

Samples of washed NSVs and AuNSVs were loaded onto 300 Cu mesh formvar carbon-coated TEM grids with a drop coating technique. For each sample, 5 or 10 μL aliquots were placed on the TEM grid and allowed to sit for 1 or 2 minutes. After 1 or 2 minutes, the excess was wicked away with a kimwipe. This process was repeated three times before the grids were left to dry overnight. TEM was carried out with a Hitachi H₇₆₀₀ TEM at 80 kV equipped with a 2k×2k camera.

### NSV Storage Stability

NSVs and AuNSVs were stored at room temperature, frozen at −20° C., or lyophilized for later use. Sucrose (0%, 5%, 10% wt/vol) was added to AuNSVs solutions before processing through the specified phase change and subsequent DLS analysis. For lyophilization, AuNSVs were frozen at −150° C. and then placed in a vial on a 4.5 L benchtop freeze dryer overnight (Labconco, Kansas City, MO). AuNSVs were then rehydrated in nanopure water or normal saline with rapid vortexing and characterized by DLS/ELS. The next day, samples were either rehydrated or thawed and measured on DLS. Repeated phase cycling was performed in 24-hour cycles at −20° C. and DLS was measured with a fresh dilution into rehydration buffer. Results were normalized to the original rehydrated value.

AuNSV absorbance through lyophilization was performed with high-concentration samples. AuNSVs were formed according to standard protocol and 100 μL of each sample (AuNSV bulk, bulk AuNSVs that had gone through the lyophilization process, and washed AuNSVs that had gone through lyophilization) were loaded onto a 96-well plate. Absorbance was measured using a SPECTROStar plate reader.

Change in zeta potential with rehydration solution was determined by measuring zeta potential of NSVs and AuNSVs rehydrated in NaCl concentrations ranging from 0 to 154 mM. Lyophilized samples were rehydrated 1:1 vol:vol in the designated saline concentration and zeta potential was measured using standard protocol.

### Cell Culture

Ovarian cancer and hepatocellular carcinoma cell lines were cultured in a biological safety cabinet under sterile conditions. TOV-112D (wt and Cav-1 kd), OV90, OVCAR5, and OVCAR8 cells were cultured in RPMI 1640 media with 10% FBS and 1X penicillin/streptomycin. Hep3B1.7 (Hep3B) cells were cultured in Dulbecco's Modified Eagle medium (DMEM) with 10% FBS and 1X penicillin/streptomycin. All cells were incubated at 37° C. in a 5% CO₂ atmosphere.

### NSV Cytotoxicity

The epithelial ovarian cancer cell line TOV-112D was cultured in RPMI 1640 media containing 10% FBS and 1% penicillin-streptomycin at 37° C. with 5% CO₂. Cells were seeded on 96-well plates in 100 μL of media at 7,500 cells per well. The next day, cells were treated in triplicate with bulk NSVs (containing ˜10% ethanol) at concentrations of 250 μg/mL, 50 μg/mL, and 10 μg/mL. After 24 hours of incubation, the media was aspirated and 110 μL of 10% CellTiter 96® AQueous One Solution Cell Proliferation Assay (MTS) (Promega, Madison, WI) in RPMI was added to each well. The cells were incubated at 37° C. and 5% CO₂ for an additional 3-4 hours before the absorbance was measured at 490 nm with a SPECTROstar plate reader. Cell viability was calculated from the ratio of absorbance between each treatment group and untreated controls.

### DOX AuNSV Cytotoxicity

TOV-112D cells were cultured in RPMI 1640 media containing 10% FBS and 1% penicillin-streptomycin at 37° C. with 5% CO₂. Cells were seeded on 96-well plates in 100 μL of media at 7,500 cells per well. The next day, cells were treated in triplicate with NSVs, AuNSVs, DOX, DOX NSVs, and DOX AuNSVs at concentrations of 0.5, 0.25, 0.1, and 0.05 μg/mL DOX equivalence. After 24, 48, and 72 hours of treatment, 10 μL of CellTiter 96® AQueous One Solution Cell Proliferation Assay (MTS) (Promega, Madison, WI) was added to each well and incubated for 3-4 hours. After incubation, the well absorbance was measured at 490 nm with a SPECTROstar plate reader with cell viability calculated from the ratio of absorbance between untreated controls and each treatment group.

### DOX Delivery

TOV-112D cells were cultured as previously described and seeded (75,000 cells per well in 1 mL media) on glass microscope cover slips rinsed in EtOH and placed in 12-well plates. The next day, cells were treated with NSVs, AuNSVs, DOX, DOX NSVs, and DOX AuNSVs at concentrations of 0.25, 0.1, and 0.05 μg/mL DOX equivalence. After 24 hours incubation, the cells were rinsed three times in PBS and then fixed in 4% paraformaldehyde in PBS (Thermo Fisher Scientific, Hampton, NH) for 20 minutes. The fixed cells were again rinsed three times in PBS, stained with DAPI, and mounted on glass microscope slides. The mounting media was allowed to dry overnight before visualization with fluorescence microscopy (Carl Zeiss Axioplan, Germany).

### GFP Live-Cell Microscopy and Flow Cytometry

TOV-112D cells were seeded overnight in 6 cm cell culture dishes (CELLTREAT Scientific Products) at a density of 300,000 cells per dish. GFP NSVs (25:1 lipid:mRNA, BioCap GFP mRNA, PhaRNA, LLC, Houston, Texas, USA) or GFP AuNSVs (2.5:1 lipid:AuNP; 25:1 lipid:mRNA, BioCap GFP mRNA) were added to cell media to a final concentration of 0.05, 0.1, or 0.25 μg/mL mRNA. Unloaded vehicles (NSVs and AuNSVs) were utilized as controls in addition to a no treatment group. After 24 hours, cells were washed in PBS 2× and visualized with live-cell fluorescence microscopy (Carl Zeiss Axioplan, Germany). Cells were then lifted with 0.25% trypsin and washed in PBS. Samples were fed into a BD FACSCelesta™ Cell Analyzer (Becton Dickinson) to collect fluorescence and scatter data, with data interpretation and graph production completed in FlowJo™ Software.

### Time-Dependent GFP Live-Cell Microscopy

TOV-112D cells were seeded overnight in 6 cm cell culture dishes (CELLTREAT Scientific Products) at a density of 100,000 cells per well. GFP NSVs (25:1 lipid:mRNA, BioCap GFP mRNA, PhaRNA, LLC, Houston, Texas, USA) or GFP AuNSVs (2.5:1 lipid:AuNP; 25:1 lipid:mRNA, BioCap GFP mRNA) were added to cell media to a final concentration of 0.1 μg/mL mRNA. Unloaded vehicles (NSV and AuNSV) were utilized as controls in addition to a no treatment group. After various time points (24, 48, or 72 hour), cells were visualized with live-cell fluorescence microscopy (Carl Zeiss Axio Observer Z1, Germany) and % of GFP+ cells was quantified using ImageJ image analysis (minimum 5 images per sample per time point).

### GFP Fixed-Cell Microscopy

TOV-112D, OVCAR8, and Hep3B were seeded overnight (25,000 cells per well in 0.5 mL media) on 12 mm glass microscope cover slips rinsed in 70% EtOH and placed in 24-well plates. Cells were then treated with GFP NSVs and GFP AuNSVs at a concentration of 0.25 μg/mL and incubated for 24 hours. Afterwards, media was aspirated, cells were washed 3× in PBS, fixed for 20 minutes at room temperature in 4% paraformaldehyde in PBS, washed 3× in PBS, stained with DAPI, and mounted on microscope slides (herein denoted as “standard fixing and staining protocol”). Slides were dried overnight and visualized with fluorescence microscopy (Carl Zeiss Axioplan, Germany) the following day. Image quantification was accomplished using an ImageJ FIJI script.

### p53 Immunofluorescence Microscopy

Hep3B cells were seeded overnight (75,000 cells per well in 1 mL media) on 12 mm glass microscope cover slips rinsed in 70% EtOH and placed in 24-well plates. Cells were then treated with p53 NSVs and p53 AuNSVs at a concentration of 1.0 μg/mL and incubated for 24 hours. Afterwards, media was aspirated, cells were washed 3× in PBS, fixed for 20 minutes at room temperature in 4% paraformaldehyde in PBS, washed 3× in PBS, and permeabilized in 0.1% Triton X100 for 20 minutes at room temperature. Slides were washed in PBS 3× again before blocking in 2% BSA in PBS for 1 hour at room temperature. After BSA was removed, slides were washed again 3× in PBS and then incubated overnight on a rocker table in 2% BSA in PBS containing p53 primary antibody (1:200, SC-126, Anti-p53 Mouse monoclonal antibody [DO-1], Santa Cruz Biotechnology). The next day, the primary antibody was removed and slides were washed 3× in PBS before incubation at room temperature with goat anti-Mouse IgG Secondary Antibody, Alexa Fluor 488 conjugate (1:1,000, Invitrogen) in PBS with 2% BSA. Secondary antibody was removed and slides were rinsed in PBS 3×, stained with DAPI, and mounted on microscope slides. Slides were dried overnight and visualized with fluorescence microscopy (Carl Zeiss Axioplan, Germany) the following day.

### Spheroid Delivery Assay

OVCAR8 cells were cultured as previously described. Non-adherent spheroid plates were made by aliquoting 50 μL of 1.5% agarose into each well of a 96-well plate. To form spheroids, 3,000 OVCAR8 cells were seeded into each well and incubated for 4 days. After 4 days, 100 μL of media was removed and 100 μL of fresh media containing Cy5 GFP NSVs or Cy5 GFP AuNSVs (0.5 μg/mL final concentration) was added. After an additional 72 hours, spheroids were imaged as a z-stack using a Zeiss Axio Observer Z1 microscope. Maximum pixel intensity (MPI) images were formed from each z-stack and used for analysis. For normalized intensity distribution, individual channel images of three spheroids were merged into a single image and Cy5/GFP intensity was plotted on a cross-sectional axis using ImageJ FIJI.

### MTS Cell Viability Assay

OV90 cells were cultured as previously described and seeded into 96-well plates (10,000 cells/well). Cells were then treated with p53 NSVs and p53 AuNSVs in 100 μL media for 24 hours at varying p53 mRNA concentration. After 24 hours, 10 μL of MTS reagent was added to each well. The cells were incubated for an additional 3-4 hours before the absorbance was measured at 490 nm with a SPECTROstar plate reader. Cell viability was calculated from the ratio of absorbance between untreated controls and each treatment group.

### ApoTox-Glo™ Triplex Assay

Viability and Caspase-3/7 activation was measured using an ApoTox-Glo™ Triplex Assay (Promega Corporation, Madison, WI, USA, cat. number G6320) according to manufacturer protocol. OVCAR8 cells were seeded overnight in 96-well plates (5,000 cells per well OVCAR8 in 0.2 mL media). After seeding, media was aspirated and replaced with 50 μL treated media (p53 NSVs or p53 AuNSVs (1.0 μg/mL), NSV/AuNSV vehicle controls, or 10 μM cisplatin). An additional 50 μL of complete media was added to each well after 6 hours and the cells were incubated for 24 or 48 at 37° C. in 5% CO₂. At each time point, 5 μL of viability/toxicity reagent was added to each well and incubated at 37° C. in 5% CO₂ for 1 hour before reading fluorescence at 400ₑₓ/505ₑₘ with a SpectroStar plate reader. After viability was determined, 25 μL of Caspase 3/7 activity reagent was added to each well, the plate was incubated on a rocker at room temperature for 30-60 minutes, and luminescence was measured using the SpectroStar plate reader. Viability was calculated as a ratio normalized to the signal produced by an untreated cell sample, and Caspase-3/7 activity was normalized to sample viability.

### Spheroid Growth Assay

OVCAR8 spheroids were formed as described above. After 4 days of spheroid formation, 100 μL of media was removed and replaced with 100 μL of media containing p53 NSVs or p53 AuNSVs (0.5 μg/mL final concentration) was added. Spheroids were imaged daily on an Olympus inverted microscope. Spheroid volume was calculated from the cross-sectional images and plotted as a function of time. After 5 days of treatment, the ApoTox-Glo™ assay was performed as previously described.

### PEGylation and Characterization of AuNPs

AuNPs were synthesized as previously described. To PEGylate the AuNPs, 1 kDa mPEG-SH (Nanocs, NY, USA) was dissolved in nanopure water to a concentration of 10 mg/mL. A 15-mL centrifuge tube was filled with 10 mL AuNP solution and 10 μL of mPEG-SH. The AuNP solution was put on the orbital shaker for 1 hour at room temperature to allow the PEG to conjugate to the AuNP surface. PEGylated AuNPs were characterized with DLS/ELS without dilution. Surface protection and PEG-AuNP concentration was determined by loading 100 μL aliquots of AuNPs and PEG-AuNPs into a 96-well plate and adding 100 μL of either nanopure water or 1.5 M sodium chloride.

Lysotracker™ Colocalization Assay with OVCAR8 Cells

Colocalization of NSVs and AuNSVs with lysosomes was assessed using fluorescence microscopy. OVCAR8 cells were seeded on 18 mm coverslips in 12-well plates at a density of 75k cells/well. Cells were treated with complete media containing 0.5 μg/mL Cy5 GFP NSVs or Cy5 GFP AuNSVs for 2, 6, or 24 hours. For the last 30 minutes of treatment, the nanoparticle-containing media was replaced with media containing 100 nM Lysotracker™ Green. After 30 minutes of Lysotracker™ exposure, cells were washed, fixed, stained, mounted, and imaged as described above. Colocalization was accomplished using an ImageJ FIJI script operating the inbuilt function EZColocalization to calculate the Pearson Correlation Coefficient (PCC) and two Mander's coefficients (M1 and M2).

### Nanoparticle Trafficking

OVCAR8 cells were seeded on 12 mm coverslips in 24-well plates at a density of 25k cells/well. Cells were treated with complete media containing Cy5 NSVs or Cy5 AuNSVs for 2 hours (samples diluted 10:1 in media and normalized to Cy5 NSV signal). After 2 hours, cells were washed and fixed according to standard protocol. Cells were then permeabilized in 0.0001% Triton X100 in PBS for 5 seconds to permeabilize outer membrane but maintain contents of intracellular vesicles. Permeabilized cells were washed 3× in PBS for 5 minutes each and then blocked in 2% BSA in PBS for 1 hour at room temperature. Blocked cells were washed in PBS 3× for 5 minutes each and then incubated in primary antibody (2% BSA in PBS; Rab7, 1:200 dilution; Cav-1, 1:50 dilution) overnight at 4° C. on a rocker. Primary antibody was washed away in PBS (3× 5 minutes) and cells were incubated in secondary antibody (2% BSA in PBS; 1:1,000 AF488-conjugated anti-mouse; 1:1,000 AF488-conjugated anti-rabbit) for 1.5 hours at room temperature. Cells were washed a final time (3× 5 minutes in PBS) before staining and mounting. Slides were imaged using a Zeiss Axio Observer Z1 and colocalization was quantified using an ImageJ FIJI script with the EZColocalization function.

p53 Delivery with RT-qPCR

Hep3B cells were seeded overnight in 6-well plates (300k per well). The next day, the media was replaced with media containing 1 μg/mL p53 mRNA delivered via p53 NSV or p53 AuNSV. Treatment was continued for 24 hours until cells were washed and lysed using a Quick-RNA MiniPrep kit following manufacturer instructions (Zymo Research, Irvine, CA, USA). Extracted RNA purity and concentration was measured using a NanoDrop spectrophotometer (ThermoFisher Scientific). mRNA was converted into cDNA in a 20 μL reaction using an iScript cDNA synthesis kit (Bio-Rad, Hercules, CA, USA) following manufacturer instructions. An iTaq Universal One-Step RT-qPCR kit (Bio-Rad) was utilized to perform RT-qPCR on a Bio-Rad CFX Connect instrument as per manufacturer instructions with custom TP53 primers and housekeeping genes GAPDH and beta actin. Relative mRNA abundance was calculated using the comparative cycle threshold method.

### Immunoblotting

Various cell lines were cultured in growth media as previously described. Cells (3×10⁵) were seeded into 6 cm dishes and incubated overnight before being dosed with NSVs and AuNSVs. The treated cells were then allowed to grow for 24 hours before lysis with RIPA buffer augmented with protease inhibitor (Pierce, Appleton, WI, USA) (1:100, v/v). Lysed cells were scraped and collected after 15 minutes in RIPA on ice; collected cell remains were retained on ice for an additional 5 minutes. Cell lysates were centrifuged at 12,500 rpm for 5 minutes before the supernatant was removed for protein quantification. Protein quantification was accomplished via Pierce BCA Protein Assay Kit (ThermoFisher Scientific, Hampton, NH) as per the manufacturer's instructions. Cell lysate volumes corresponding to 5-20 mg protein were subsequently incubated at 95° C. for 5 minutes in Laemmli buffer with β-mercaptoethanol and loaded onto freshly-prepared 10% tris-glycine SDS-polyacrylamide gel electrophoresis gels before transfer to polyvinylidene difluoride membranes. Membranes were blocked with 5% skim milk in TBST (Tris Buffered Saline with Tween 20) for 1 hour at room temperature before incubation overnight with primary antibody overnight at 4° C. After incubation with the primary antibody (mouse anti-p53 1:1,000, rabbit anti-GAPDH 1:50,000, mouse anti-alpha tubulin 1:10,000), membranes were washed 3× in TBST before 1 hour incubation at RT with secondary antibodies (1:10,000). Membranes were rinsed an additional 3× and then dipped in Clarity Western ECL substrate (BioRad Laboratories, Hercules, CA, USA) for 3 minutes before drying and image capture via X-ray. Developed films were scanned and band intensity was quantified using ImageJ FIJI (National Institutes of Health) with GAPDH for normalization.

### BrdU Proliferation Assay

Cell proliferation was measured using an Abcam BrdU Cell Proliferation ELISA Kit (colorimetric, ab126556). Hep3B cells were seeded in 96-well plates at 2,000 cells per well. Cells were treated with media containing both BrdU and either p53 NSVs (1 μg/mL), p53 AuNSVs (1 μg/mL), or equivalent vehicle controls for 24 hours. Cisplatin (10 μM) was used as a positive control. After 24 hours, cells were fixed and BrdU incorporation was determined using manufacturer protocol.

### Cell Cycle Assay

Hep3B cells were seeded in 6-well plates at a density of 200k cells per well. Cells were treated with for 24 hours in serum-free media according to assigned treatment group (no treatment control, NSV or AuNSV vehicle control, p53 NSV (0.25 μg/mL), p53 AuNSV (0.25 μg/mL), or 10 UM cisplatin). After 2 hours, 3.5× volume of complete media was added to each well and the cells were incubated for an additional 24 hours. Cells were lifted with 0.25% trypsin, washed in PBS and fixed in ice-cold 70% ethanol for 1 hour. Fixed cells were washed once in PBS and stained with FxCycle™ PI/RNase Staining Solution (ThermoFisher Scientific, cat. number F10797). Scatter and fluorescence intensity data was collected with a FACSCelesta flow cytometer and analyzed with FlowJo software.

### TUNEL Assay

Hep3B apoptosis was assessed using a DeadEnd™ Fluorometric TUNEL System (Product G3250) according to manufacturer protocols. Hep3B cells were seeded and treated on 12 mm glass coverslips as described in the immunoblotting protocol. Treated cells on 12 mm coverslips were placed in a 24-well plate and washed and fixed using the standard protocol. After fixation, cells were permeabilized in 0.1% Triton X100 for 5 minutes at room temperature, washed in PBS, and apoptotic cells were stained using the manufacturer instructions. Coverslips were mounted with DAPI and imaged using fluorescence microscopy with analysis performed using an ImageJ FIJI script.

### Colony Formation Assay

The clonogenic capacity of Hep3B cells was assessed using a colony formation assay. Hep3B cells were counted and diluted to a concentration of 200 cells/mL. Diluted cells were separated into 12 mL aliquots and treated according to designation (no treatment, 0.1 mg/mL p53 NSVs, 0.1 mg/mL p53 AuNSVs). Treated cells were dispensed into 6-well plates (3 wells with 4 mL cells in each). After 10 days of incubation, the media was removed and cells were stained and fixed in 20% methanol containing 0.1% crystal violet for 2 hours at room temperature. Stained cells were washed once in deionized water and dried overnight. Plates were imaged using a GelCount™ colony scanner (Oxford Optronix, UK) and images were analyzed using an ImageJ FIJI script.

### AuNSV Uptake Rate Assay

AuNSV uptake rate was measured using flow cytometry. TOV-112D cells were seeded into 6-well plates at a density of 300k cells/well. Cell were treated with complete media containing 5 μg/mL equivalent of NBD-labeled NSVs or AuNSVs. At each time point (10 minutes, 30 minutes, or 2 hours), the cells were washed 3× in ice-cold PBS and lifted with 0.25% trypsin. Cells were washed 1× in ice-cold PBS and kept on ice until scatter and fluorescence data was collected on the FACSCelesta instrument. After the initial run, cell samples were stained with 0.67% trypan blue (1:5 vol:vol ratio of 4% stock) to quench surface fluorescence and allow differentiation between surface-associated and cell endocytosed nanoparticles. Surface quenched samples were run on the FACSCelesta as before and fluorescence was analyzed using FlowJo software.

### AuNSV Uptake Pathway Assay

Uptake pathways were assessed using fluorescence microscopy. TOV-112D (wt and Cav-1kd), OVCAR8, Hep3B, and OVCAR5 cells were seeded in 24-well plates on 12 mm coverslips (25k-50k cells/well; some replicates performed using 18 mm coverslips in 12-well plates at 75k cells/well). The next day, cells were pretreated for 30 minutes with inhibitors in serum free media: chlorpromazine (CPZ, 10 μg/mL), methyl-β-cyclodextrin (MβCD, 5 mM), filipin (5 μg/mL), 5-(N-Ethyl-N-isopropyl) amiloride (EIPA, 0.025 mM), or sodium azide (NaN₃, 20 mM). After pretreatment, serum-free media was replaced with complete media containing 5 μg/mL equivalent of Cy5-labeled NSVs or AuNSVs. Treatment was carried out for 30 minutes or 2 hours when cells were fixed and mounted according to standard protocol. Cells were imaged at 64× (40× objective, 1.6× optical zoom, 5 images per sample) with a Carl Zeiss fluorescent microscope and images were quantified using an ImageJ FIJI script with Cy5 signal normalized to DAPI.

The biological uptake inhibition assay was performed in a similar manner. TOV-112D cells (wt and Cav-1 kd) were seeded on 12 mm coverslips in 24-well plates. Cells were exposed to Cy5 GFP NSVs or Cy5 GFP AuNSVs (0.5 μg/mL) for 24 hours before the cells were washed, fixed, stained, and imaged as previously described.

### Lysotracker™ Colocalization Assay

Colocalization of NSVs and AuNSVs with lysosomes was assessed using fluorescence microscopy. TOV-112D cells were seeded on 12 mm coverslips in 24-well plates at a density of 25k cells/well. Cells were treated with complete media containing 0.5 μg/mL Cy5 GFP NSVs or Cy5 GFP AuNSVs for 23.5 hours. After 23.5 hours, the nanoparticle-containing media was replaced with media containing 50 nM Lysotracker™ Red. After 30 minutes of Lysotracker™ exposure, cells were washed, fixed, stained, mounted, and imaged as described above. Colocalization was accomplished using an ImageJ FIJI script operating the inbuilt function EZColocalization to calculate the Pearson Correlation Coefficient (PCC) and two Manders' coefficients (M1 and M2).

### AuNP Association Assay

TOV-112D cells were cultured as previously described and lysed in Pierce IP Lysis buffer containing 1× protease inhibitor cocktail for 15 minutes on ice. Cell debris was removed with centrifugation at 12,500 rpm for 5 minutes and protein concentration was determined using a Pierce™ BCA assay. AuNP and PEGylated AuNP samples were loaded into 1.5 mL microcentrifuge tubes (25 μg each) and 100 μg protein lysate was added to each tube. Samples were incubated for at least 24 hours at room temperature on a rotator to allow for protein corona formation. Corona composition was assessed by spinning down the AuNPs (10k rpm, 20 minutes). The AuNP pellets were washed 3× in ultrapure water before being stored on ice while the supernatant fractions were concentrated on an Eppendorf Vacufuge™ for 2 hours at 30° C. Samples were diluted in loading buffer, heated, and then loaded onto 12% SDS-PAGE gels in the following volumes: 5 μL raw lysate, 25 μL concentrated supernatant, total pellet fractions. Immunoblotting was carried out as previously described with primary antibodies for Rab7 (1:1,000) and PP2Ac (1:1,000).

### PP2A Activation Assay

PP2A activity was measured using a PP2A Immunoprecipitation Phosphatase Assay Kit (Sigma-Aldrich, catalog 17-313). TOV-112D cells were cultured in 10 cm dishes until they reached 60% confluency. Culture media was replaced with complete media containing the vehicle equivalent of 1 μg/mL mRNA-loaded NSVs or AuNSVs. Cells were treated for 24 hours and washed 3× in ice-cold Tris-Buffered Saline (TBS). Cells were lysed for 15 minutes on ice in IP Lysis buffer containing 1× protease inhibitor cocktail and cell debris was subsequently pelleted by centrifuging at 12,500 rpm for 5 minutes. Protein concentration was determined using BCA assay. PP2A pulldown was performed according to protocol instructions using 500 μg protein, 25 μL Protein A agarose, and 4 μL Anti-PP2Ac antibody for 30 minutes at 4° C. on a rotator. Beads were washed and phosphatase reactions were carried out for 10 minutes at room temperature on a rotator with 30 μL phosphopeptide and 70 μL assay buffer in each tube. Samples were then spun down and 25 μL were added to each well on a 96-well plate with 75 μL malachite green solution. The reaction was read at 650 nm after 10 minutes incubation at room temperature with relative phosphatase activity normalized to NSV-treated cells.

### Rab7 Activation Assay

Rab7 activation was measured using a protocol published by the Li lab with a pGEX-4T3 GST-RILP plasmid that was a kind gift from Dr. Mark McNiven of the Mayo Clinic. Briefly, GST-RILP was produced through induction of transformed BL-21 cells and purified on glutathione beads. TOV-112D cells were treated with vehicle equivalents of 1 μg/mL mRNA delivery vectors for 24 hours before washing, lysing, and pulldown of active Rab7 according to the aforementioned protocol (500 μg lysate with 30 μL GSH-GST-RILP beads). Immunoblotting was performed as described above with Rab7 primary antibody (1:1,000) and band intensity was calculated in ImageJ FIJI.

### Animal Housing

Male and female athymic nude mice (5-6 weeks old) were purchased from Charles River Laboratories (Houston, TX, USA). Animals were housed in pathogen-free conditions in OUHSC facilities approved by the American Association for Accreditation of Laboratory Animal Care and in compliance with all regulations supplied by the USDA, DHHS, and NIH. The animal use protocol was reviewed and approved by the OUHSC Institutional Animal Care and Use Committee (IACUC).

### Tumor Growth Studies

Hep3B cells were cultured as described previously. Cells were suspended in PBS and injected into the flank of athymic nude mice (107 per animal) to form subcutaneous tumors. Equal numbers of male and female mice were randomly assigned to treatment groups (saline, AuNSV, p53 NSV, or p53 AuNSV; n=8 per group). Treatment commenced upon a tumor volume threshold of 100 mm³ (V=tw²/2=l=tumor length, w=tumor width as measured by calipers). Animals received 3× weekly injections (8 total) of 0.5 mg/kg p53 mRNA or equivalent controls and tumor volume was measured as a function of time. Animals were sacrificed on day 26 post initial injection if they had not met IACUC-mandated endpoints (tumor volume>1000 mm³, tumor length>1.5 cm, or quality of life standards). Tissues were collected and tumor mass was recorded before preservation at −150° C. or in 10% neutral buffered formalin.

### Statistics

Data analysis was performed in GraphPad Prism 10. All results are calculated as mean±standard error from at least three individual experiments unless otherwise stated. The statistical significance of differences between two independent and normally distributed groups was determined using an unpaired two-tailed t test with Welch's correction. Differences between several experimental groups and one control group were calculated using one-way ANOVA with Dunnett's multiple comparisons test, while significant relationships between all groups in an experiment were calculated using ordinary one-way or two-way ANOVA with Tukey's multiple comparisons test. Unless stated otherwise, significant p values are indicated in figures and/or legends as * p<0.05, ** p<0.01, *** p<0.001, **** p<0.0001.

### Results

### NSV Development and Characterization

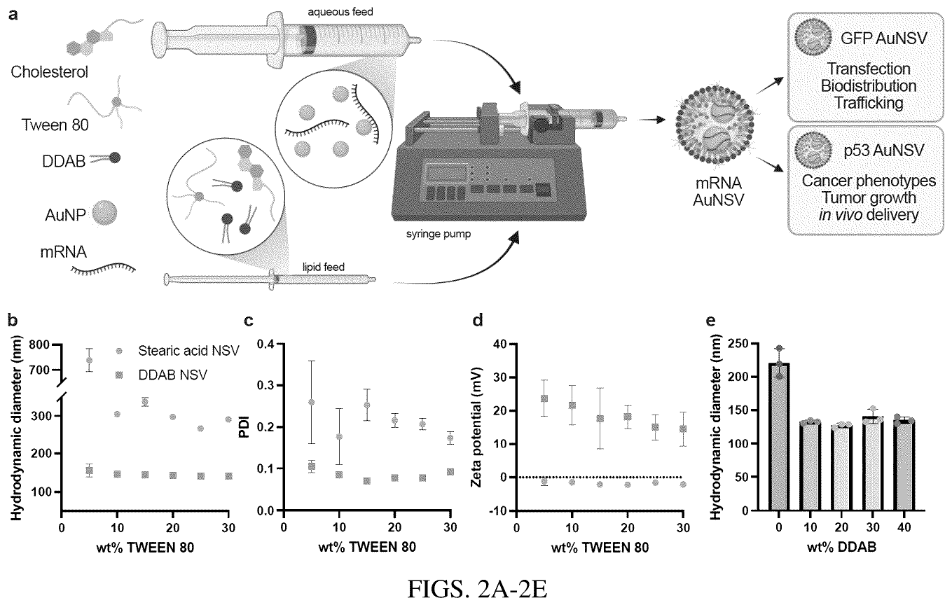

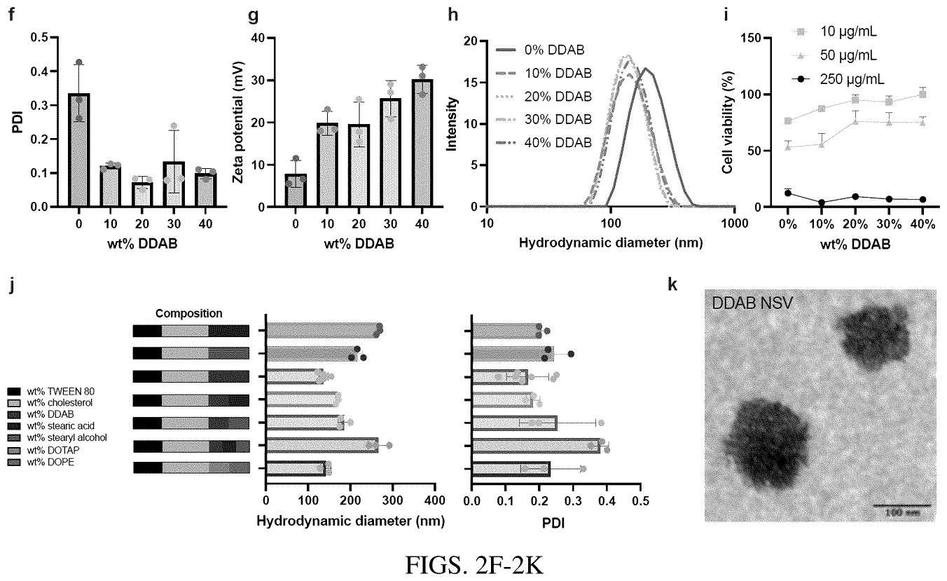

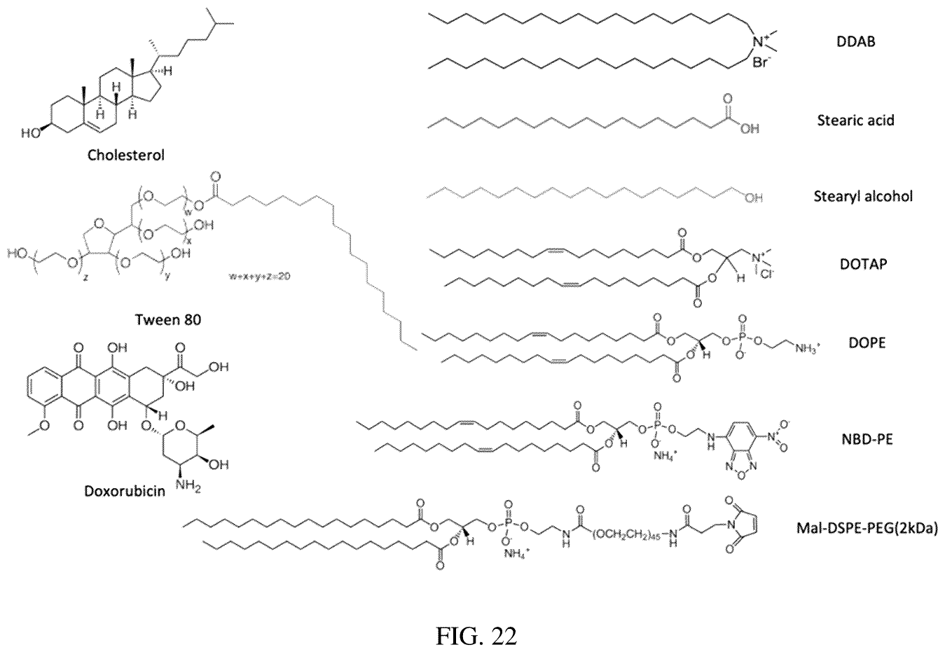

The millifluidic synthesis process was developed with the aim of producing a system capable of continuous production of tunable non-ionic surfactant vehicles. All materials were selected for chemical stability, previously observed applications in delivery of biologics, or prior shown efficacy in conjunction with AuNPs (Oliveira et al., ACS Appl Mater Interfaces 6, 6977-6989 (2014); Patel et al., J Control Release 303, 91-100 (2019); Patel et al., Nature Communications 11, 983 (2020); Azhari et al., Eur J Pharm Biopharm 104, 148-155 (2016); Han et al., Journal of Nanobiotechnology 19, 394 (2021); Zhao et al., Nanoscale 2, 2114-2119 (2010)) (FIG. 22). Succinctly, a lipid stream containing varying ratios of lipid, cholesterol, and TWEEN 80® (T80) was fed at 500 μL/min into a millifluidic tube where it joined an aqueous stream of sodium acetate buffer (25 mM, pH 4) flowing at 4.2 mL/min. The millifluidic tubing ran through an ultrasonic bath where ultrasonic cavitation induced formation of lipid nanoparticles (NSVs). Dimethyldioctadecylammonium bromide (DDAB) and stearic acid were selected for initial NSV development as model cationic and anionic lipids respectively. Dynamic light scattering (DLS) results showed that NSV size decreased with increasing T80 until a minimum particle size was observed at 25 wt % T80 composition (FIG. 2B-D) for both LNPs formed from DDAB and stearic acid. For stearic acid NSVs, PDI generally decreased with increasing T80 whereas DDAB NSVs displayed a PDI of 0.1, which suggests highly monodisperse nanoparticles. From these data, 25 wt % T80 was selected for further NSV formulations.

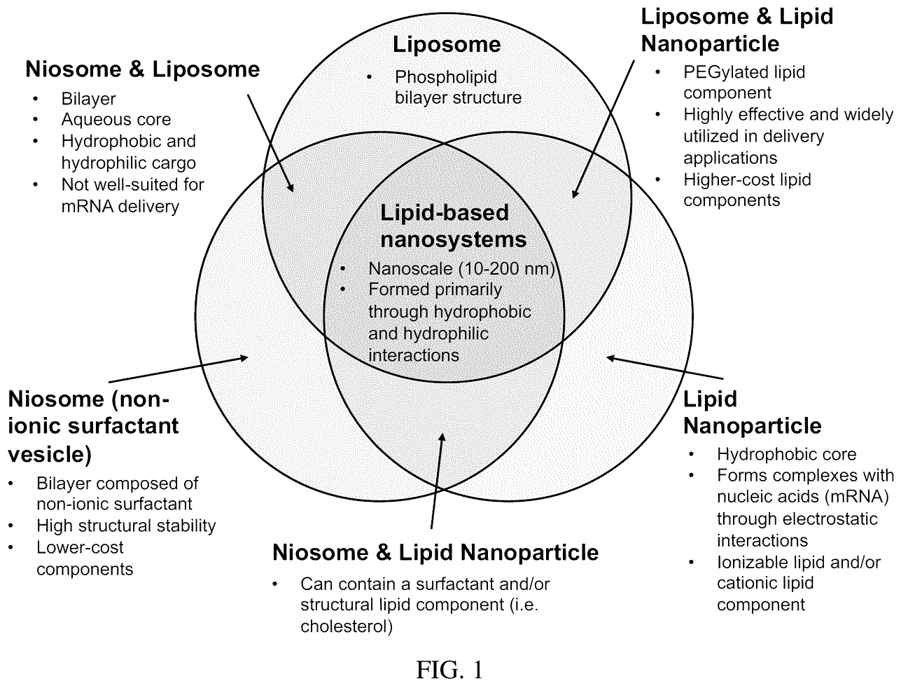

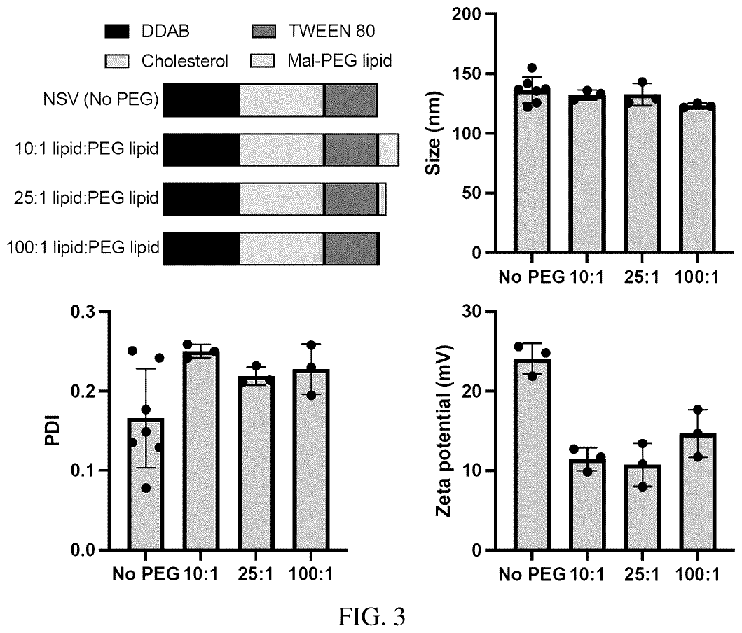

After verifying the NSV composition of T80, we varied the ratios of DDAB and cholesterol and observed the changes in nanoparticle size and charge (FIG. 2E-G). Size and PDI were relatively consistent for NSVs with 10-40% DDAB, while zeta potential increased with increasing DDAB content. Each displayed tight DLS intensity peaks (FIG. 2H). To ensure NSV biocompatibility, TOV-112D ovarian cancer cells were exposed to 250 μg/mL, 50 μg/mL, and 10 μg/mL concentrations of NSVs with viability measured after 24 hours (FIG. 2I). Interestingly, cell viability increased with decreasing NSV concentration and increasing DDAB content, suggesting that increasing zeta potential does not correlate to reduced cell viability. Next, varying cationic, anionic, and neutral lipids were utilized in conjunction with cholesterol and T80 to produce several NSV formulations (FIG. 1J). Those containing cationic lipids were generally smaller and monodisperse in comparison to those incorporating neutral and anionic components, with DDAB displaying the most promising DLS characteristics. From the aforementioned data, we selected 25% T80, 40% cholesterol, and 35% DDAB as the lipid mixture to be the basis for all subsequent NSV compounds. TEM displayed particles with amorphous lipid cores that present high potential for loading of therapeutics (FIG. 2K) (Sharma et al., Polymer Bulletin 78, 2553-2567 (2021); Makoni et al., Pharmaceutics 11, 397 (2019)). Later studies confirmed the case of NSV modification with maleimide-functionalized PEGylated lipids through inclusion in the lipid stream mixture for later addition of targeting moieties (FIG. 3).

### Development and Characterization of AuNSVs

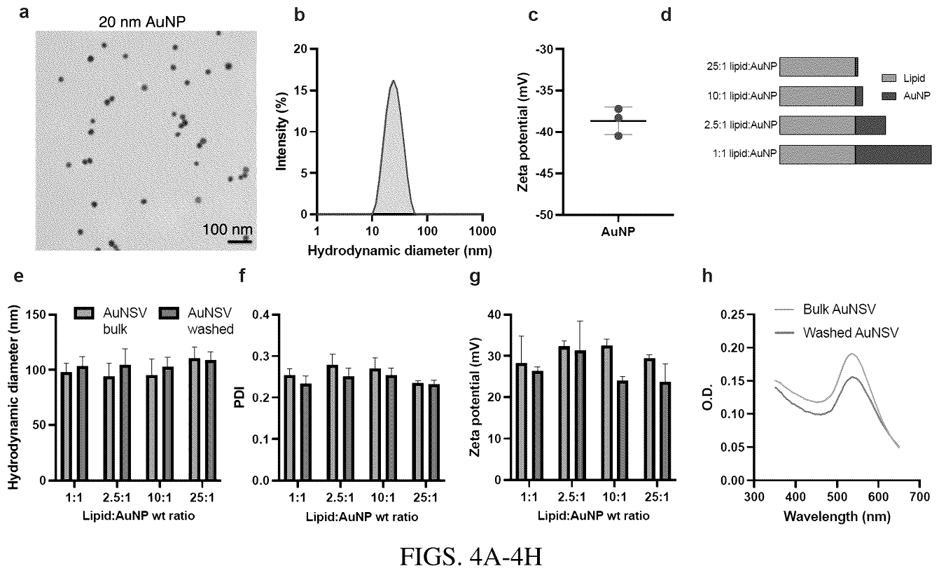

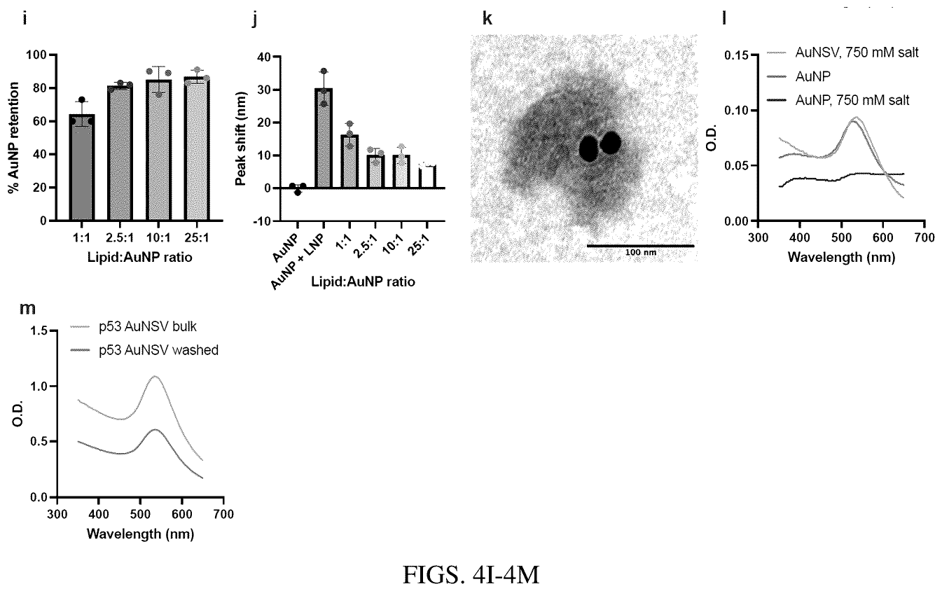

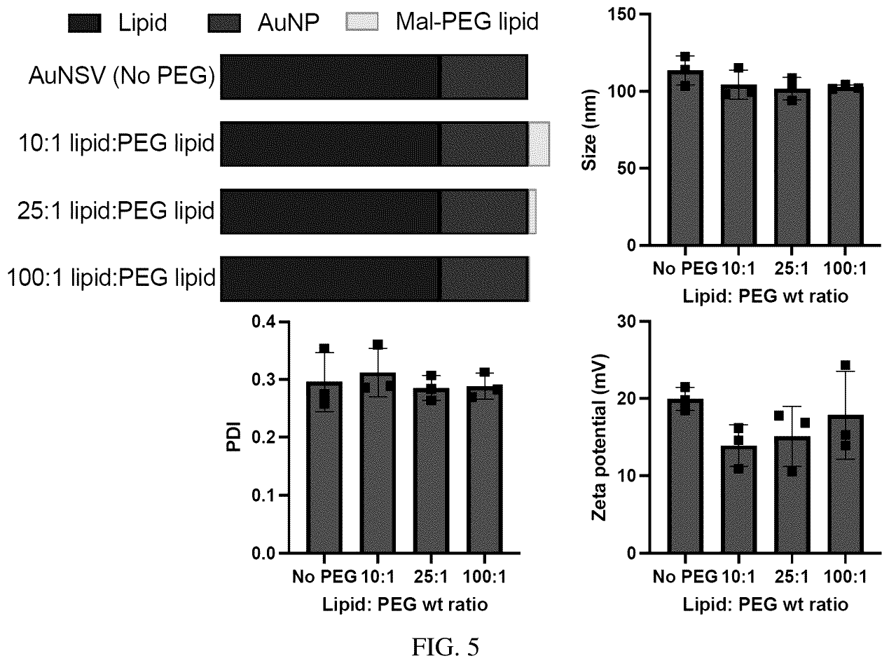

Following optimization of lipid composition, NSVs were doped with AuNPs to produce auro-non-ionic surfactant vehicles (AuNSVs). AuNPs (20 nm diameter) were synthesized from chloroauric acid (FIGS. 4A-C) and fed to the system in sodium acetate buffer at lipid:AuNP mass ratios of 1:1, 2.5:1, 10:1, and 25:1 (FIG. 4D). The bulk AuNSV product was washed using a 30 kDa concentrator to remove unassociated lipids and AuNPs. The size, PDI, and zeta potential of the bulk and washed samples were determined via DLS (FIG. 4E-G). The bulk and washed AuNSVs were observed to be around 100 nm in diameter with a PDI of 0.25, and incorporation of varying amounts of AuNPs had little effect on the AuNSV zeta potential with all washed samples falling between +25 and +35 mV. As the bare AuNPs utilized in the study exhibit a negative zeta potential (FIG. 5), this suggests that AuNPs are retained within the NSV structure rather than associated with the NSV surface.

AuNP retention was characterized by absorbance spectroscopy as calculated by the ratios of the washed to bulk AuNSV absorbance peak at 534 nm (FIGS. 4H-I). AuNP retention increased from 60% to 85% with increasing lipid:AuNP ratio due to increased lipid availability. To further identify the location of AuNPs within the AuNSV structure, the peak absorbance wavelength of AuNSVs with varying compositions was compared to those of the AuNPs alone and a mixture of AuNPs with pre-formed NSVs (FIG. 4J). AuNPs alone displayed an absorbance peak of 524 nm while the NSV-AuNP mixture had an absorbance peak of 555 nm. Greater peak shifts were observed with lower lipid content which suggests greater surface association of AuNPs. AuNSVs with higher lipid content displayed smaller peak shifts, which suggests fewer surface-associated AuNPs. TEM visualization of AuNSVs showed AuNPs retained within the homogenous core of the lipid nanomaterial (FIG. 4K).

Without stabilizers, AuNPs aggregate in the presence of high salt concentrations as observed by loss of the characteristic AuNP absorbance peak; consequently, AuNSVs were next exposed to 750 mM sodium chloride to identify whether the presence of lipids retained the colloidal stability of AuNPs (FIG. 4L). The AuNP absorbance peak was maintained for each of the AuNSV compositions. Moreover, the absorbance peak was retained when AuNPs were mixed with pre-formed NSVs; this evidence supports the hypothesis that NSVs stabilize both encapsulated and surface-associated AuNPs, which could be beneficial for AuNPs to enact self-therapeutic effects in biodistribution and at cellular membranes.

AuNSVs of varying composition were utilized throughout the remainder of the studies. Drug encapsulation studies utilized a 25:1 lipid:AuNP ratio to maximize loading capacity, whereas mRNA delivery studies utilized a 2.5:1 lipid:AuNP ratio as this was identified to maximize delivery efficacy. p53-mRNA-loaded AuNSVs retained 60% of AuNPs as compared to the bulk product (FIG. 4M), which is consistent with the loading of mRNA. As with NSVs, AuNSVs were readily modified with maleimide-functionalized PEGylated lipids to alter AuNSV properties for specific applications (FIG. 5).

### NSV and AuNSV Storage and Stability

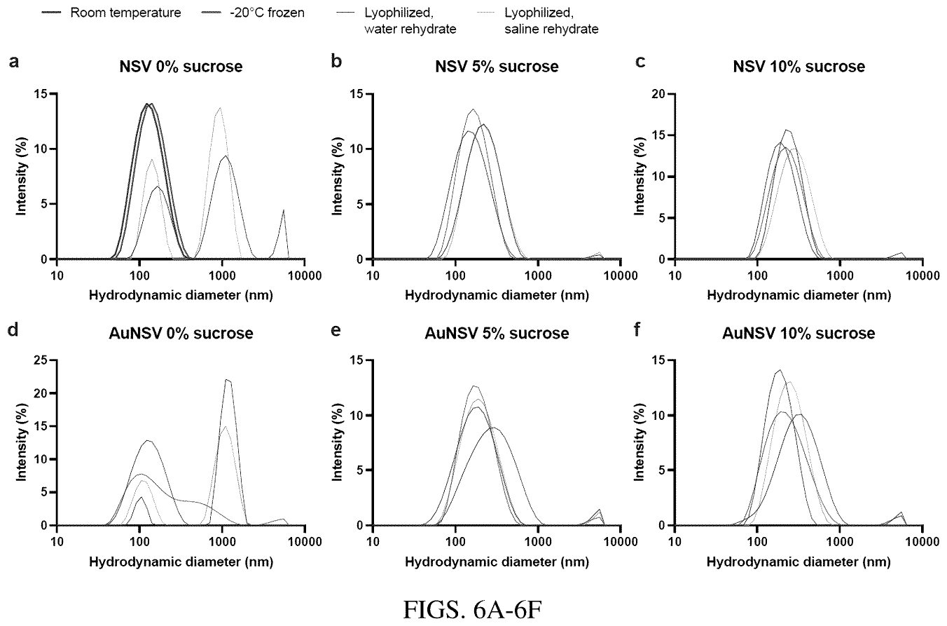

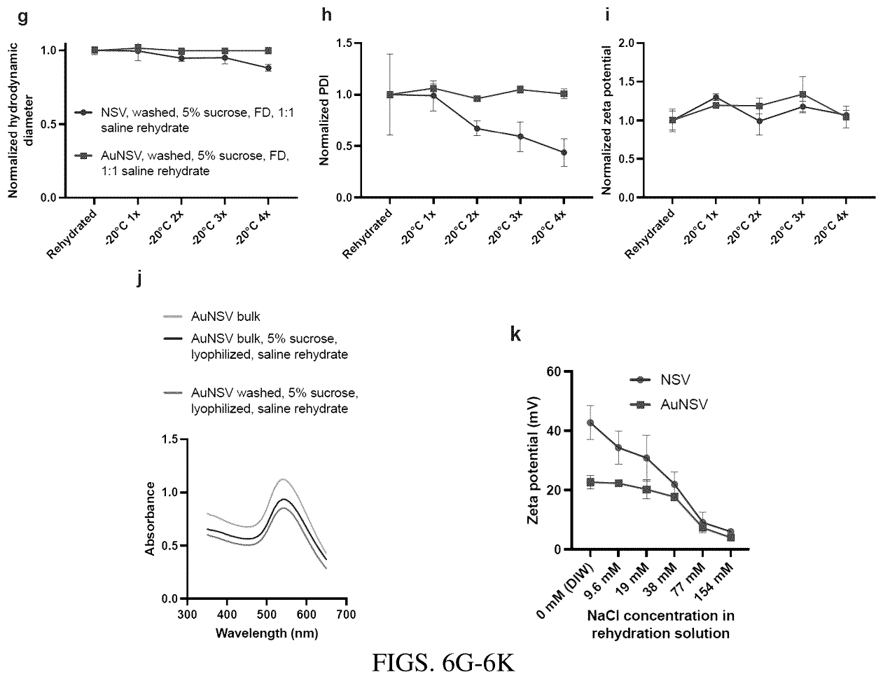

After determining the components and ratios for the NSV and AuNSV formulations, we endeavored to determine optimum storage conditions for maintaining physicochemical characteristics (FIG. 6A-F). NSV and AuNSVs were stored at room temperature, frozen at −20° C., and lyophilized at −80° C. with rehydration in either nanopure water or normal saline. While NSVs alone maintained hydrodynamic diameter and PDI through both room temperature storage and single cycle freezing, AuNSVs began aggregation through the freeze/thaw cycle. Moreover, both NSVs and AuNSVs aggregated dramatically and lost monodispersity when rehydrated after lyophilization. Sucrose was dissolved in samples (5% and 10% wt/vol) to serve as a cryoprotectant by forming a macromolecular cage surrounding AuNSVs to prevent aggregation and maintain physicochemical properties (Lee et al., J Pharm Sci 98, 4808-4817 (2009)). Both NSVs and AuNSVs retained monodispersity through phase changes when combined with 5% or 10% sucrose. The concentration of sucrose affected both the size and zeta potential of NSVs and AuNSVs, with higher concentrations corresponding to increased size and reduced zeta potential. This is intuitive, as sucrose will electrostatically associate with the cationic surface of NSVs and AuNSVs and produce these effects.

The stability of NSVs and AuNSVs through successive phase changes was studied using DLS. NSVs and AuNSVs (2.5:1 lipid:AuNP ratio) were synthesized using a protocol to produce concentrated particles that were then washed, stabilized with 5% sucrose, lyophilized, and stored at −20° C. Samples were rehydrated in normal saline and cycled through 4 phase changes at −20° C. with DLS measurements recorded after each (FIG. 6G-I). Size and PDI measurements remained consistent for AuNSVs across all cycles, whereas size and PDI of NSVs trended downwards with increasing number of freeze/thaw cycles. All of these characteristics remained within the range desired for useful nanotherapeutics, which suggests that AuNSV-delivered drugs and biologics may respond favorably to re-freezing after initial rehydration.

AuNP retention throughout the lyophilization process was measured with absorbance spectroscopy. Using identically-processed NSV samples as blanks, high concentration AuNSVs (2.5:1 lipid:AuNP ratio) retained the AuNP absorbance peak through lyophilization and rehydration (FIG. 6J). A 17% decrease in absorbance was observed between bulk product and bulk product that had been lyophilized, and an additional 7% reduction was observed between the bulk and washed lyophilized products. This provides evidence that only a small fraction of AuNPs are lost in the lyophilization process. Based on these data, subsequent experiments were conducted with either freshly synthesized AuNSVs, AuNSVs stabilized with 5% sucrose and stored at −20° C., or AuNSVs stabilized with 5% sucrose, lyophilized, and stored at −20° C. until being rehydrated.

Lyophilized AuNSVs responded to rehydration in normal saline better than in nanopure water, and thus saline was selected as the rehydration solvent for later studies. Swapping the solvent from sodium acetate to saline also reduced the AuNSV zeta potential from +25 to +35 down to +5 to +10 (FIG. 6K). A near neutral zeta potential is beneficial for extension of circulation time and reduction in cytotoxicity, and thus saline rehydration of lyophilized AuNSVs may be advantageous for in vivo use.

### Doxorubicin-Loaded AuNSVs

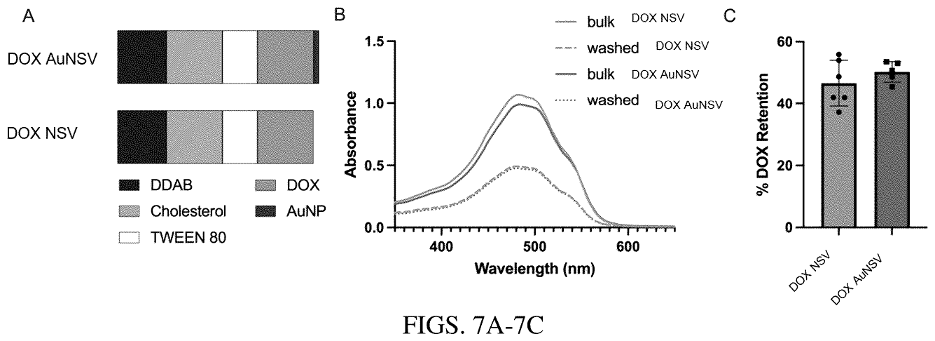

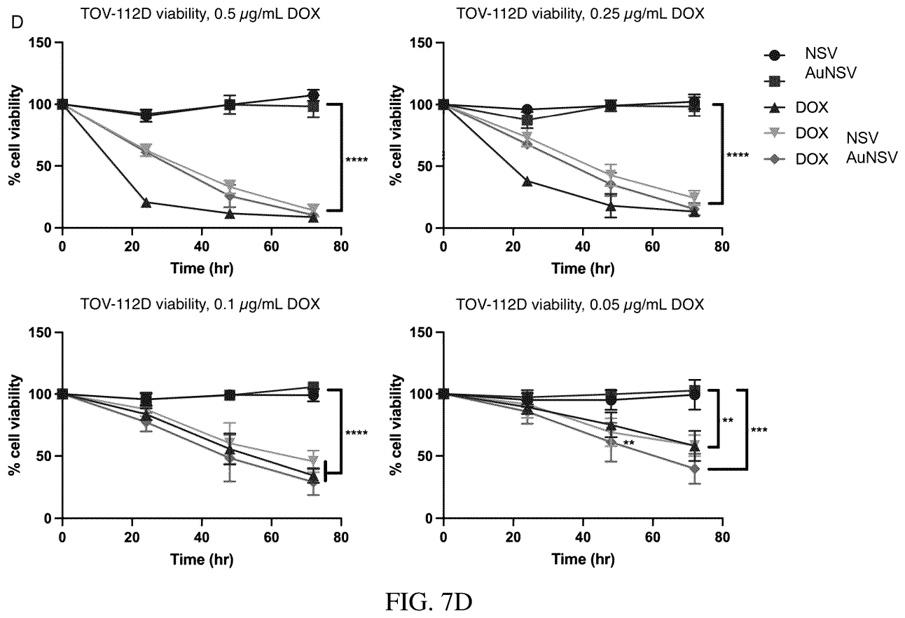

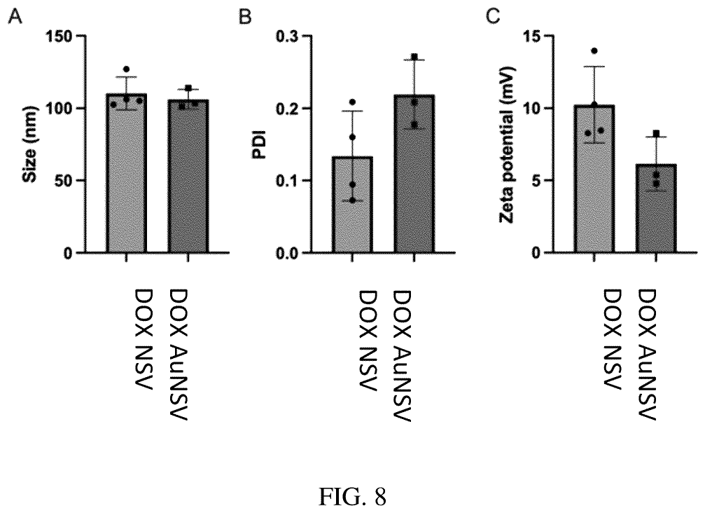

Once AuNSV design parameters and storage conditions had been elucidated, AuNSVs were loaded with small-molecule therapeutics. Doxorubicin (DOX) was loaded in the lipid stream at a 2.5:1 lipid:DOX ratio (FIG. 7A). After washing, each formulation was characterized for size, PDI, and zeta potential (FIGS. 7A-C), and encapsulation efficiency determined by comparison of the absorbance peak at 490 nm (FIGS. 7B-C). Each sample displayed sizes slightly greater than 100 nm with corresponding monodispersity and DOX loading efficiency of 50% (FIG. 8). With confirmation of DOX loading, NSVs were doped with AuNPs to create DOX AuNSVs with a lipid:AuNP ratio of 25:1. This lipid:AuNP ratio was selected to maximize the available loading capacity of the DOX AuNSVs, and these dual-loaded nanoparticles displayed similar loading efficiency and average size as DOX NSVs.

The in vitro efficacy of DOX AuNSVs was determined using a cell viability assay (FIG. 7D). TOV-112D cells were treated with DOX, DOX NSVs, or DOX AuNSVs and cell viability was quantified at 24-, 48-, and 72-hour time points. Unloaded NSVs and AuNSVs displayed minimal cytotoxicity at all time points at all concentrations observed. At 0.5 μg/mL and 0.25 μg/mL, free DOX decreased cell viability faster than either the DOX NSV or DOX AuNSV. As DOX concentrations decreased to 0.1 μg/mL and 0.05 μg/mL, however, DOX NSVs display similar cytotoxicity to free DOX. Moreover, DOX AuNSVs showed improvement over both free DOX and DOX NSVs at 0.05 μg/mL by displaying increased significance in difference between vehicle controls and treatment groups. Though no statistically significant differences were observed in comparison to treatment groups, this observation is consistent with previous reports of AuNPs improving the efficacy and delivery of chemotherapeutics (Seaberg et al., ACS Nano 15, 2099-2142 (2021); Huai et al., Cell Stress 3, 267-279 (2019)). It also further supports the hypothesis that AuNPs promotes delivery efficacy by altering cellular uptake and trafficking.

mRNA AuNSV Development and Characterization

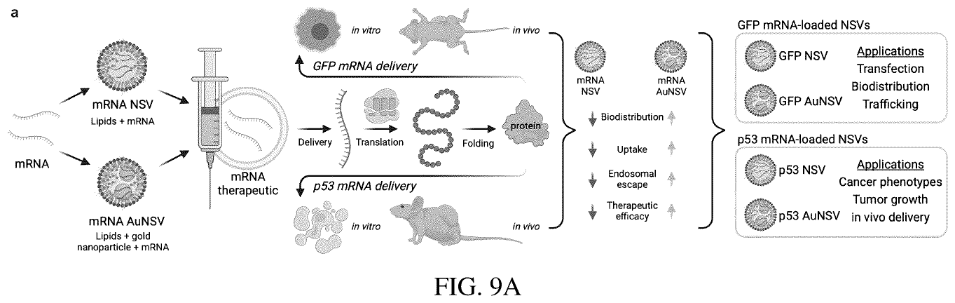

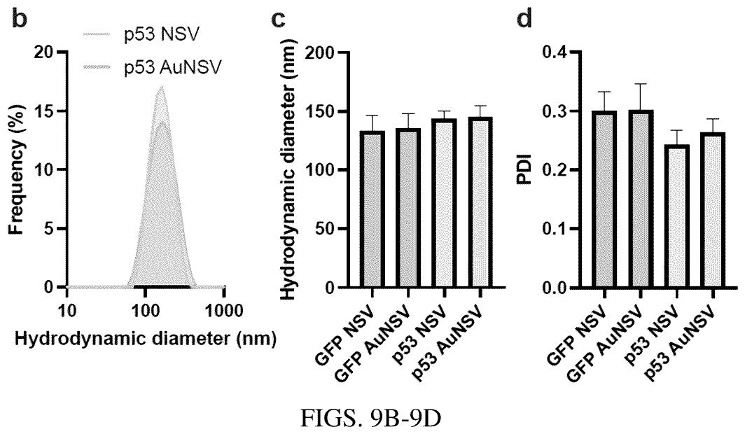

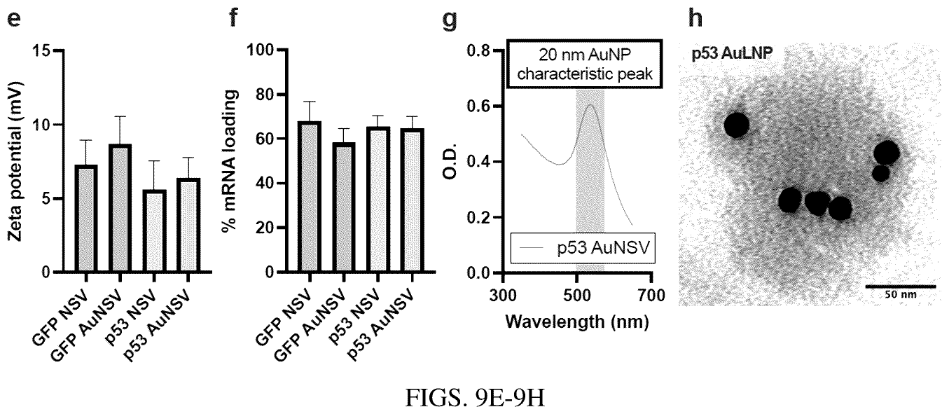

We designed four NSV variants to study the effect of 20 nm AuNPs on mRNA delivery characteristics. mRNA coding for green fluorescent protein (GFP) was loaded either directly into NSVs or co-loaded with AuNPs to create GFP NSVs and GFP AuNSVs, while mRNA coding for the tumor suppressor p53 was loaded into NSVs and AuNSVs to form p53 NSVs and p53 AuNSVs. GFP NSVs and GFP AuNSVs were employed in transfection, biodistribution, and trafficking studies, while p53 NSVs and p53 AuNSVs were used to study tumor growth and cancer hallmarks (FIG. 9A). Physicochemical characterization revealed monodisperse nanoparticles with consistent hydrodynamic diameters approaching 150 nm, PDI of 0.3, a slightly cationic zeta potential, and no reduction in mRNA loading with co-encapsulation of AuNPs (FIG. 9B-G). AuNSVs retained the absorbance peak above 500 nm that is characteristic of 20 nm AuNPs, confirming that AuNPs do not aggregate throughout the synthesis process (FIG. 9G). Transmission electron microscopy revealed an amorphous spherical structure with AuNPs dispersed throughout (FIG. 9H). The similarity in physicochemical properties of the mRNA NSVs and AuNSVs substantiates that differences in delivery efficiency are not due to bulk characteristics. All NSVs and AuNSVs were created with a proprietary millifluidic synthesis apparatus and were highly stable through extended storage periods and repetitive freeze/thaw cycles. Extensive materials selection, characterization, and storage stability are provided in FIGS. 2, 4, 6, and a detailed synthesis procedure is outlined in the methods.

AuNSVs Enhance Translation of GFP mRNA in Cancer Cells

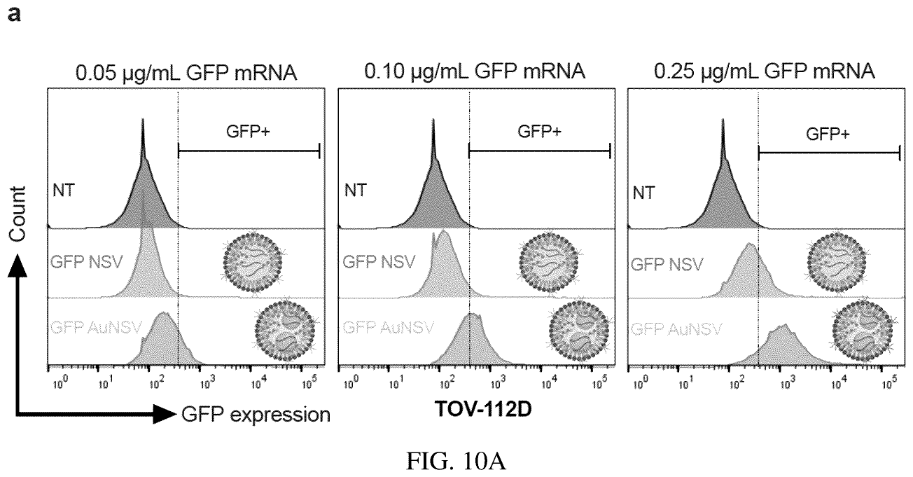

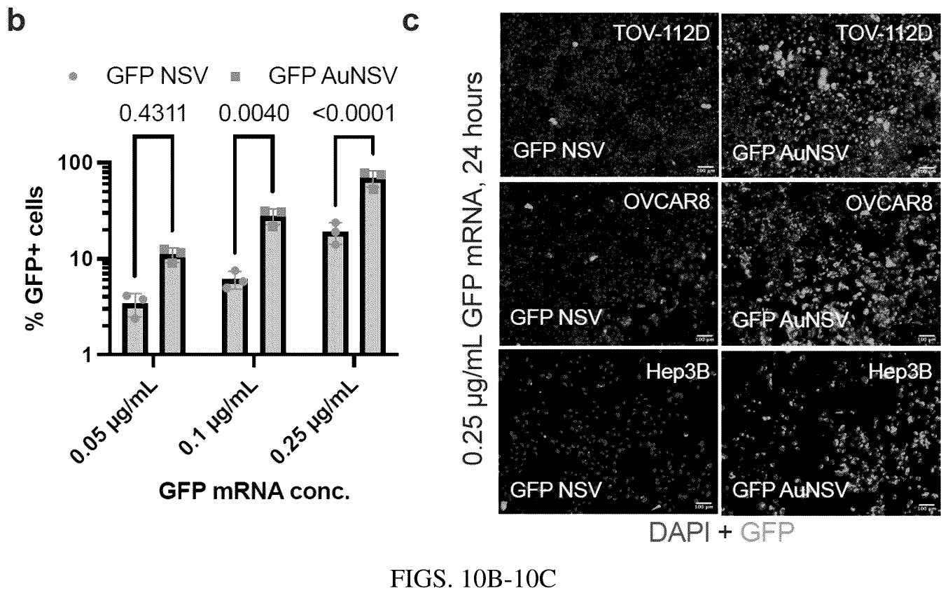

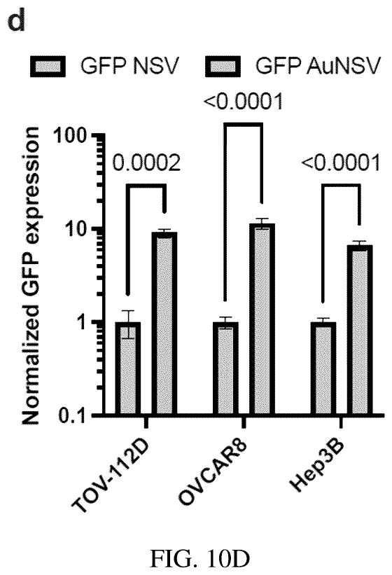

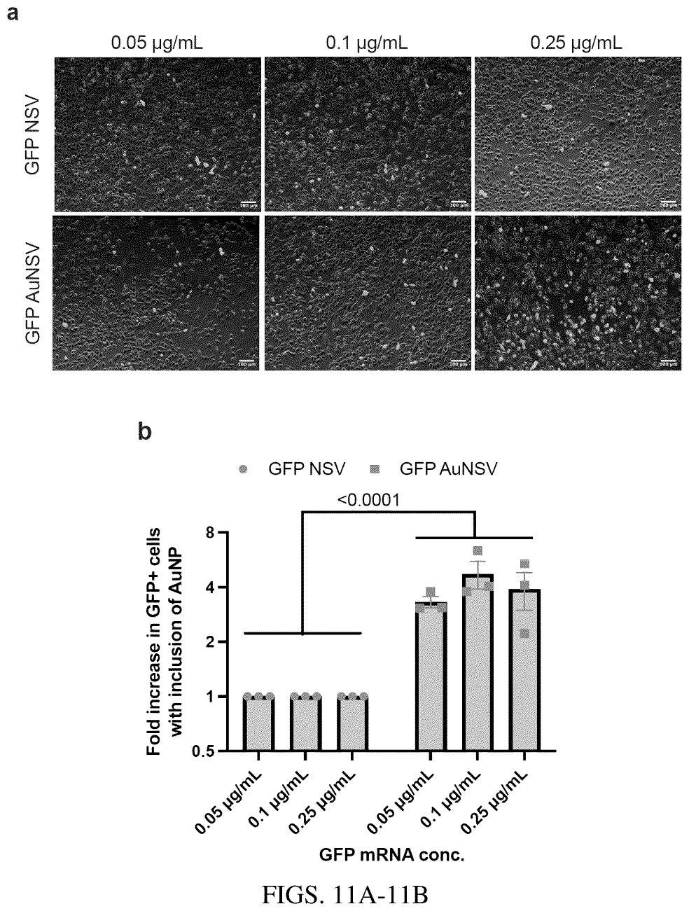

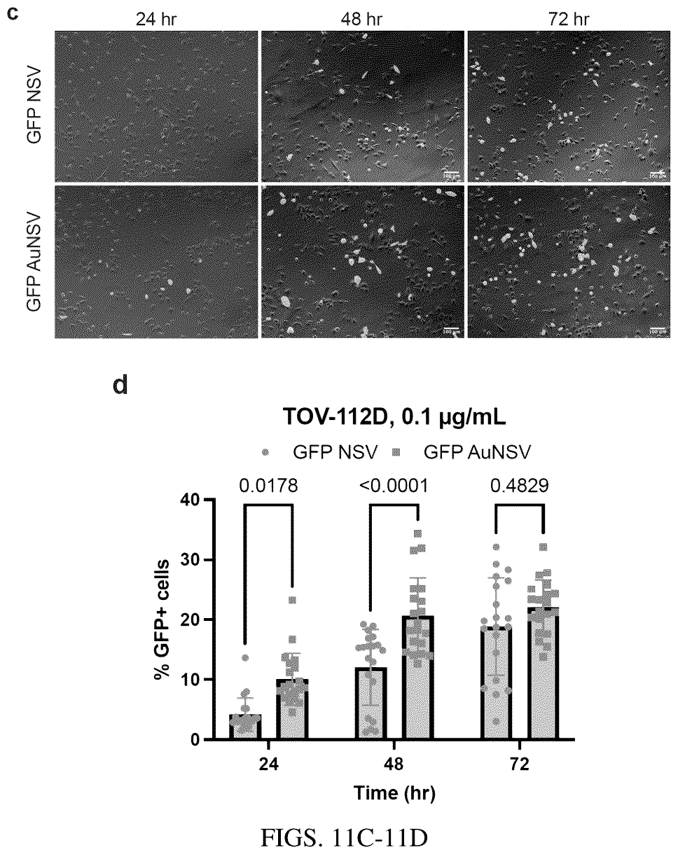

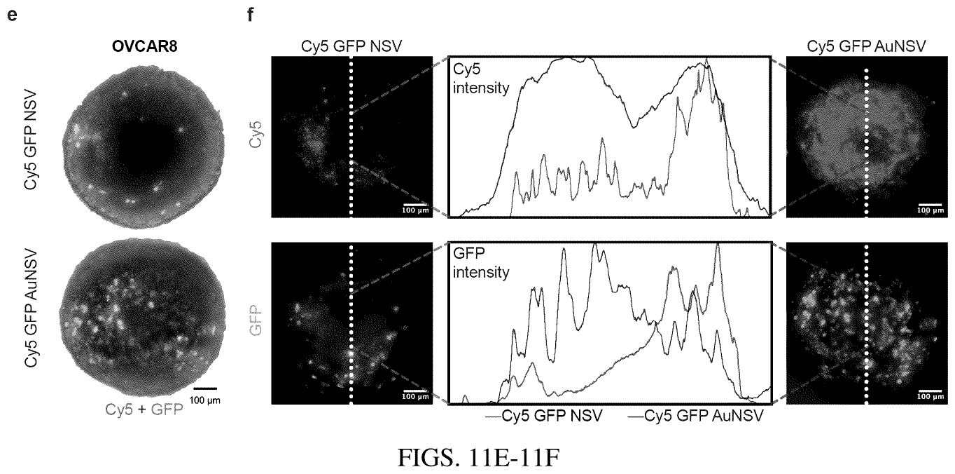

To determine how AuNPs affect the delivery and translation of mRNA in cancer cells, we treated TOV-112D ovarian cancer cells for 24 hours with varying concentrations of GFP mRNA delivered in GFP NSVs or GFP AuNSVs and measured the fluorescence intensity of the translated protein with flow cytometry. At low concentrations there was a moderate increase in GFP expression in cells treated with GFP AuNSVs in comparison to GFP NSVs (FIGS. 10A, 11A-B). As concentration increased, the GFP expression of cells treated GFP AuNSVs increased at a greater rate than did those treated with GFP NSVs. At 0.25 μg/mL, just over 10% of the TOV-112D cells in the GFP NSV group were GFP+, whereas the GFP AuNSV group was composed of nearly 75% GFP+ cells (FIG. 10B).

To ascertain the generalizability of this finding, we treated three cancer cell lines (TOV-112D, ovarian cancer, p53-mutant; OVCAR8, ovarian cancer, p53-mutant; Hep3B, hepatocellular carcinoma, p53-null) with GFP NSVs or GFP AuNSVs and observed them under fluorescence microscopy (FIG. 10C). For each cell line, GFP AuNSV treatment displayed a consistently greater GFP expression treatment with GFP NSVs did (FIG. 10D). Among these cell lines we selected p53-null Hep3B cells for tumor reduction, biodistribution, and cancer hallmark studies along with TOV-112D cells for mechanistic experiments. OVCAR8 cells were utilized in both mechanistic and in vitro therapeutic studies with data presented as supplemental information.

GFP expression over time in TOV-112D cells was studied using fluorescence microscopy. GFP NSVs and GFP AuNSVs were added to TOV-112D cells in culture (0.1 μg/mL) and visualized with fluorescence microscopy after 24-, 48-, and 72-hours (FIG. 11). Low magnification micrographs of live cells showed a significant increase in GFP+ cells with GFP AuNSV treatment in comparison to GFP NSV treatment after 24 hours (FIG. 11). This difference was maintained but to a lesser extent at the 48-hour time point, while there were no differences in quantity of GFP+ cells after 72 hours. From these data we observed a 3× increase in GFP+ cells with GFP AuNSV treatment in comparison to GFP NSV treatment at 24 hours along with a nearly 2× increase after 48 hours (FIG. 11). Taken together, these data suggest that AuNP-doping improves mRNA delivery through promotion of rapid uptake and endosomal escape, allowing for earlier maximum expression of protein within transfected cells.

AuNSV delivery in 3D cell culture was studied using an OVCAR8 spheroid model. Cancer spheroids exposed to 0.5 μg/mL Cy5-labeled GFP NSVs displayed reduced Cy5 signal and GFP expression in comparison to those treated with 0.5 μg/mL Cy5 GFP AuNSVs (FIG. 11E). We also looked at composite figures and observed more uniform distribution and expression of both fluorophores in Cy5 GFP AuNSV-treated spheroids in comparison to NSV counterparts (FIG. 11F).

AuNSV Delivery of p53 mRNA Activates Apoptosis in p53-Null Hep3B Cancer Cells

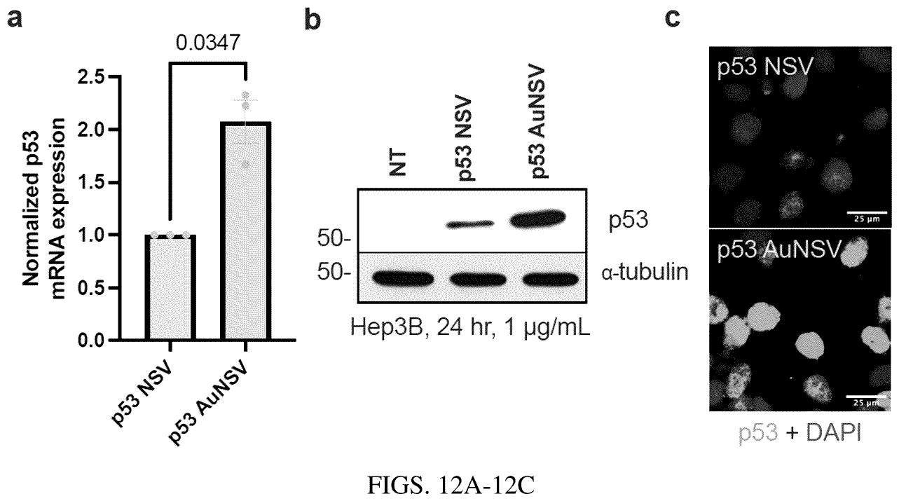

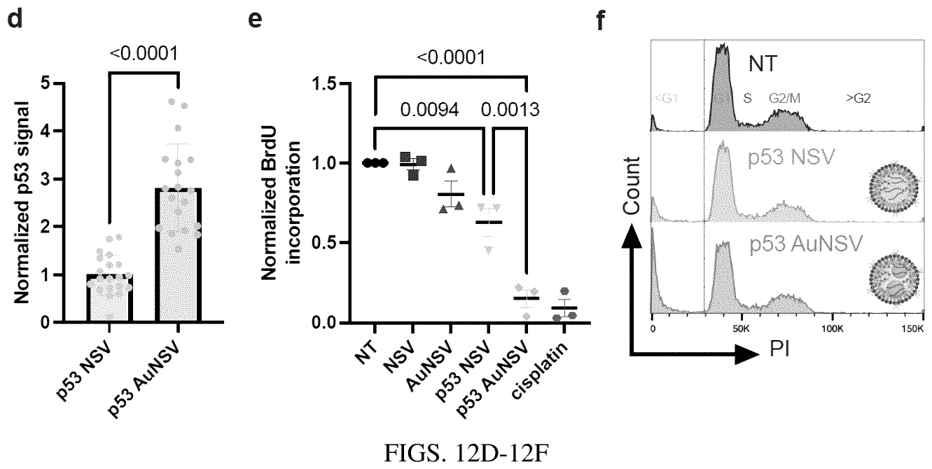

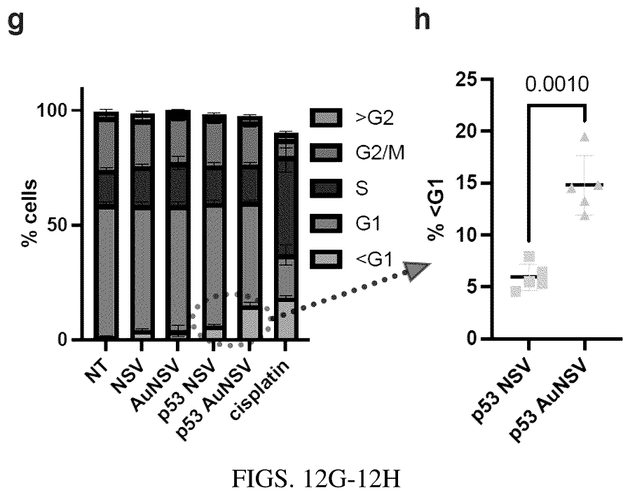

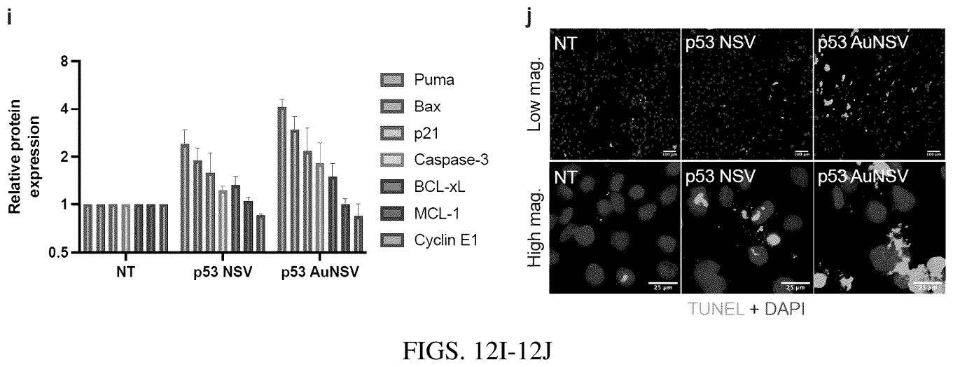

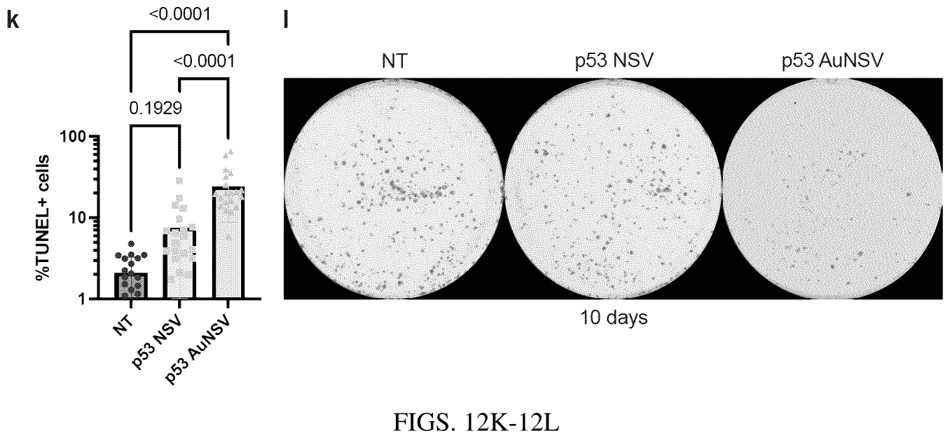

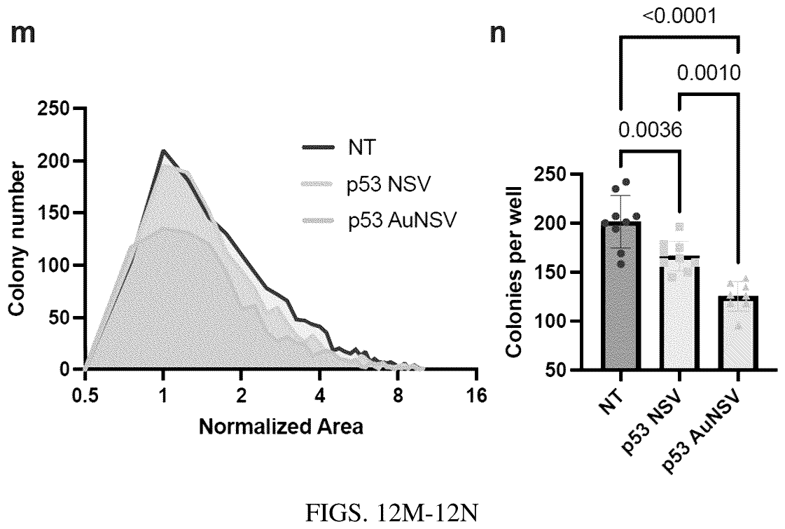

With the proof of concept for enhanced mRNA delivery to cancer cells via AuNSV established, we next sought to deliver therapeutically relevant mRNA. We selected delivery of tumor-suppressing p53 mRNA to p53-null Hep3B cells as a model system to understand the effects of AuNSV-mediated delivery in cancer-relevant mRNA delivery systems. When treated for 24 hours, Hep3B cells exposed to p53 AuNSVs displayed twice the relative expression of p53 mRNA as did cells treated with p53 NSVs (FIG. 12A). Western blot and immunofluorescence imaging confirmed that this increase in p53 mRNA corresponded to an incrementally greater increase in p53 protein expression (FIGS. 12B-D, 10A-B), suggesting that AuNPs play a role in both mRNA vector uptake and in protein translation.

We next examined how restoration of wild-type p53 function altered the functional phenotypes displayed by p53-null Hep3B cells. We initially observed that p53 AuNSVs hindered Hep3B cell proliferation to a significantly greater extent than did p53 NSVs or vehicle controls (FIG. 12E). As p53 can activate both apoptosis and cell cycle arrest, we sought to identify the extent to which these pathways are activated by delivery of exogenous p53 mRNA. Using cell cycle analysis, we noted a significantly increased apoptotic sub-G1 population in Hep3B cells treated with p53 AuNSVs in comparison to p53 NSVs (FIG. 12F-H). Interestingly, we did not observe the G1 phase holdup expected with p53-mediated cell cycle arrest. Western blot analysis of pro-apoptotic (Puma, Bax, Caspase-3), anti-apoptotic (BCL-xL, MCL-1), pro-cell-cycle arrest and/or senescence (p21), and anti-cell-cycle arrest (Cyclin E1) markers downstream from p53 revealed an upregulation of pro-apoptotic and -cell-cycle arrest/senescence markers without a corresponding decrease in anti-apoptotic and -cell-cycle arrest proteins (FIG. 12I). This suggests that p53 AuNSV therapy activates apoptosis rapidly through a flood of pro-apoptotic signals that outcompetes modulating signals and alternative p53-regulated signaling cascades.

We selected two end-stage phenotypic assays to assess the therapeutic effect of p53 AuNSV therapy. We performed a TUNEL assay and observed a significantly increased proportion of apoptotic cells among those treated with p53 AuNSVs in comparison to p53 NSVs (FIG. 12J, K). We also quantified the clonogenic capacity of Hep3B cells exposed to treatment with p53 NSVs and p53 AuNSVs through a colony formation assay (FIG. 12L). After 10 days of incubation, p53 AuNSV treatment reduced both the size and number of colonies in comparison to p53 NSV treated cells (FIG. 12M, N).

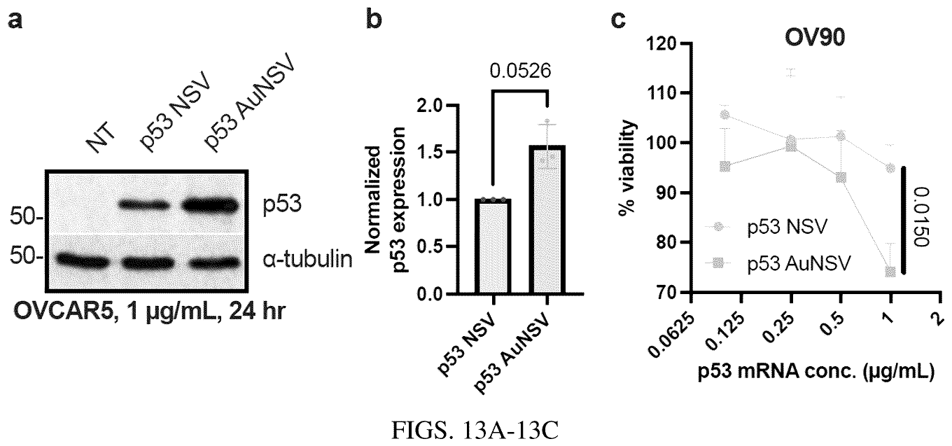

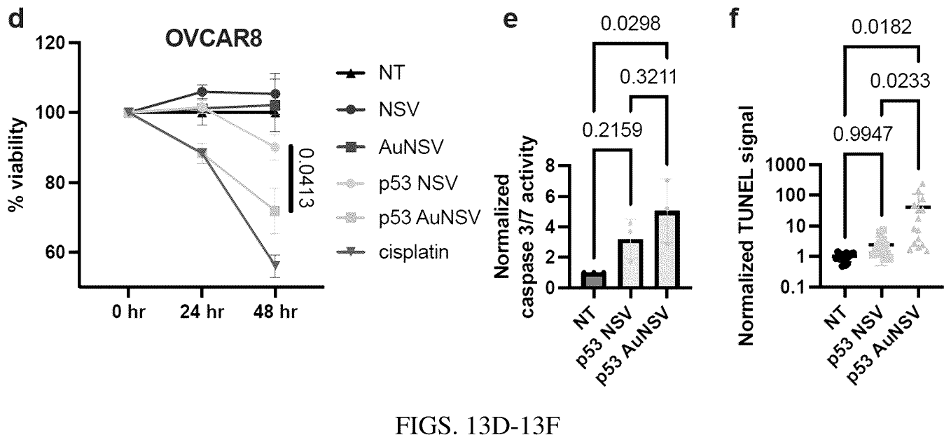

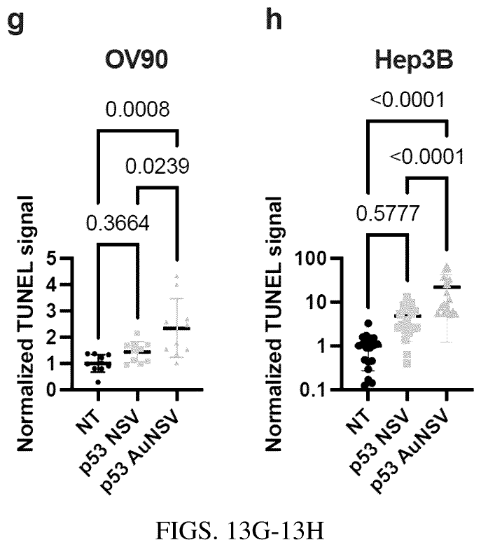

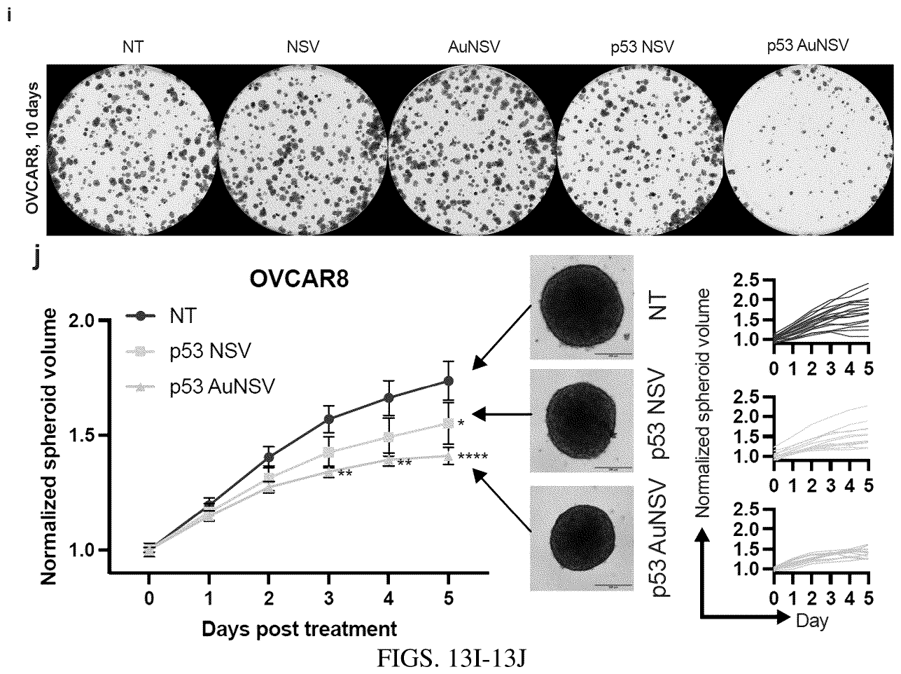

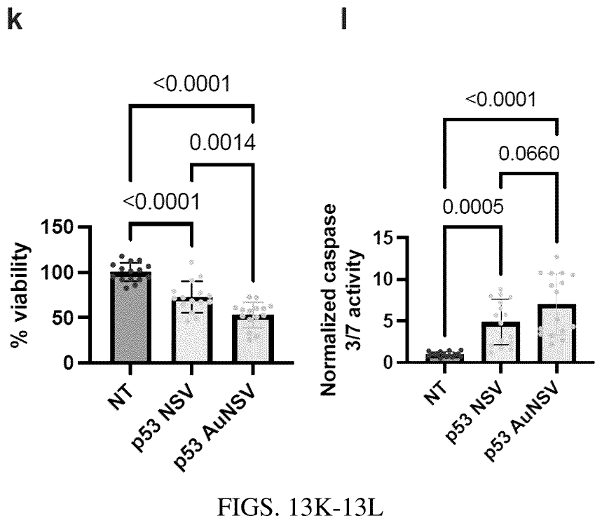

The delivery and efficacy findings in Hep3B cells was further generalized to ovarian cancer cell lines exhibiting various types of p53 mutations (FIG. 13). Delivery of p53 to OVCAR5 cells showed a substantial increase with p53 AuNSVs in comparison to p53 NSVs. Both OV90 and OVCAR8 cells showed decreased viability due to p53 AuNSV treatment that is primarily due to activation of apoptosis. Again, we expanded from 2D culture to 3D culture to observe the effects of p53 AuNSV therapy on OVCAR8 spheroids and observed significant reductions in viability and increases in caspase activity with p53 AuNSV treatment. These findings further strengthen the claim that AuNSV mRNA delivery is a generalizable therapeutic strategy.

AuNSVs Improve Efficacy of p53 mRNA Therapy in p53-Null Hep3B Tumor Models

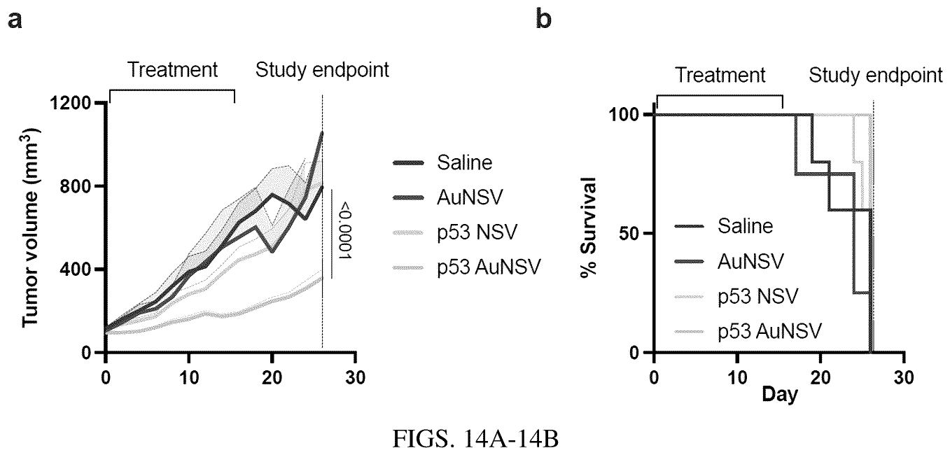

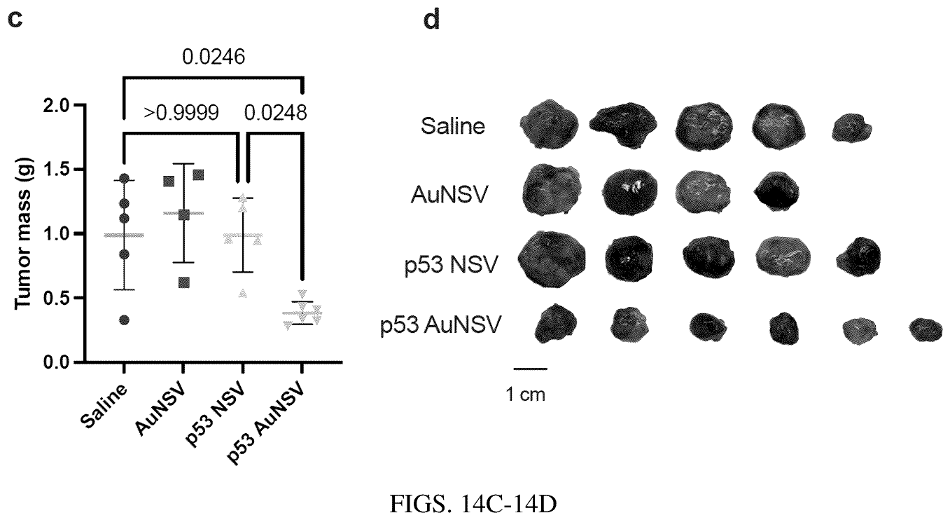

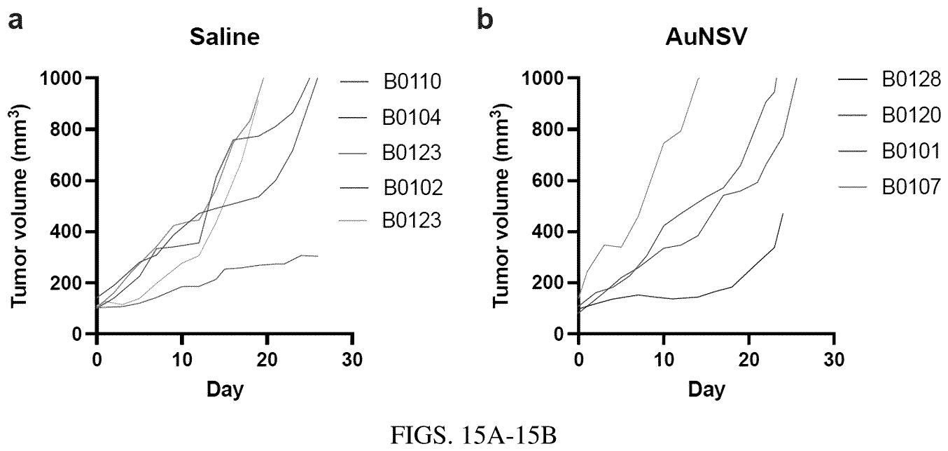

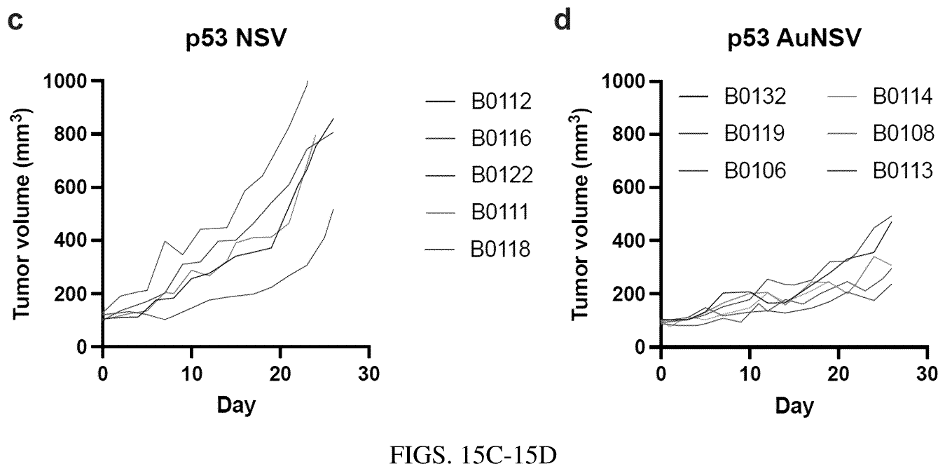

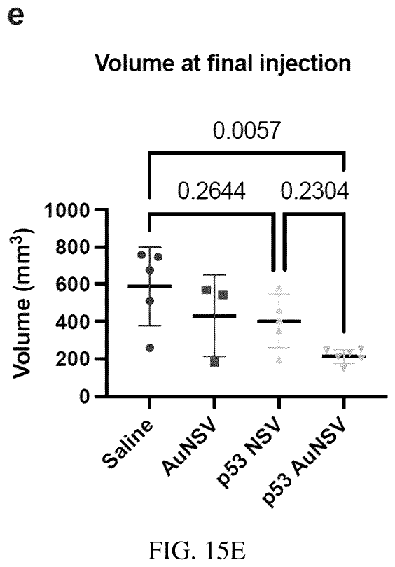

With the in vitro efficacy of p53 AuNSV therapy validated, we next sought to translate these findings in vivo. We subcutaneously inoculated athymic nude mice with p53-null Hep3B cancer cells and randomized them to one of four groups (saline control, AuNSV vehicle control, p53 NSV treatment, or p53 AuNSV treatment). Once tumors reached 100 mm³, treatment was initiated and tumor volume was measured as a function of time. Each animal received 8 injections (3× week) and final tumor mass was measured upon sacrifice or on completion of the study (Day 26). We observed greater tumor growth inhibition and improved survival in the p53 AuNSV treatment group in comparison to p53 NSV treatment group (FIG. 14A-B, 15A-D). Isolated tumors treated with p53 AuNSVs were significantly smaller than those in any other treatment group (FIG. 14C-D).

### AuNSVs Enhance Vector Uptake in Cancer Cells by Preferentially Activating Caveolae-Mediated Endocytosis

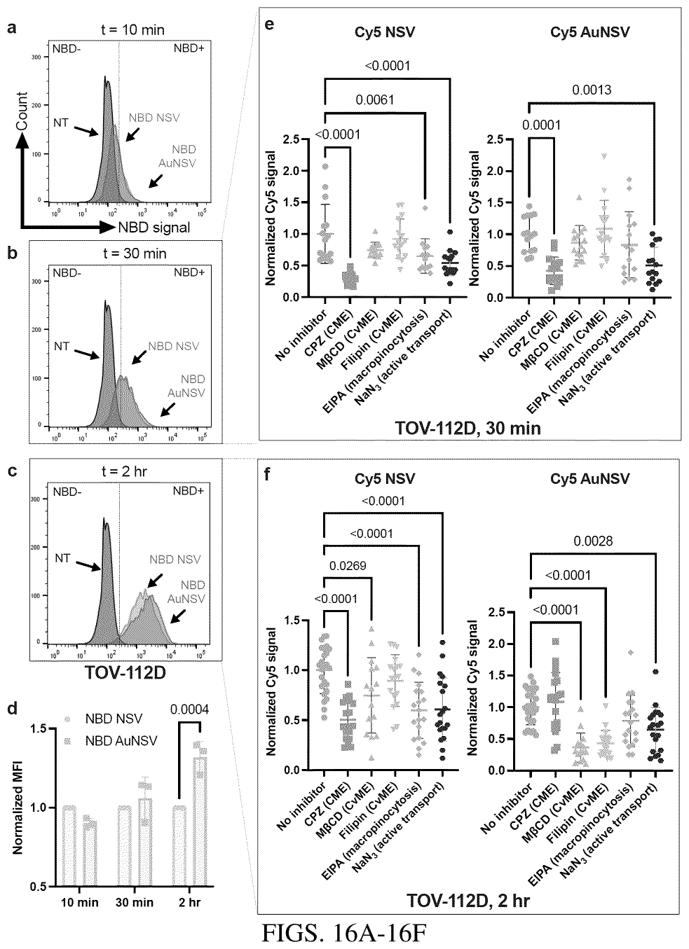

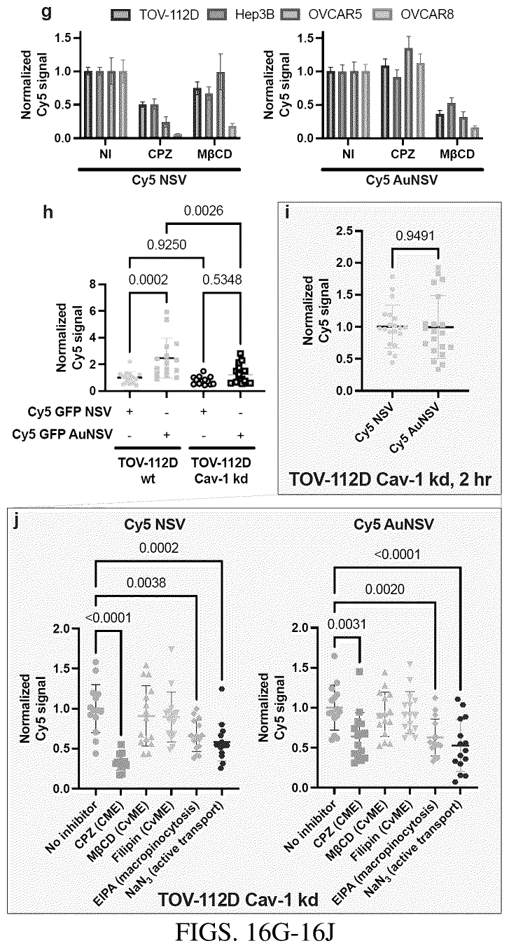

Once we established the advantages of AuNP-augmented mRNA delivery, we sought to understand its mechanism. To accomplish this task, we designed NSVs and AuNSVs incorporating Cy5- or NBD-fluorophore-conjugated lipids to allow for spaciotemporal tracking of each vector. Initial studies in TOV-112D cells showed no difference in mean fluorescence intensity (MFI) or NBD+ cell population after 10-minute or 30-minute delivery of NBD NSVs or NBD AuNSVs (FIG. 16A, B). After two hours of delivery, however, there was a significant increase in MFI in cells treated with NBD AuNSVs in comparison to those treated with NBD NSVs (FIG. 16C, D). This suggests that between 30 minutes and 2 hours there is an AuNP-dependent change in nanoparticle uptake.

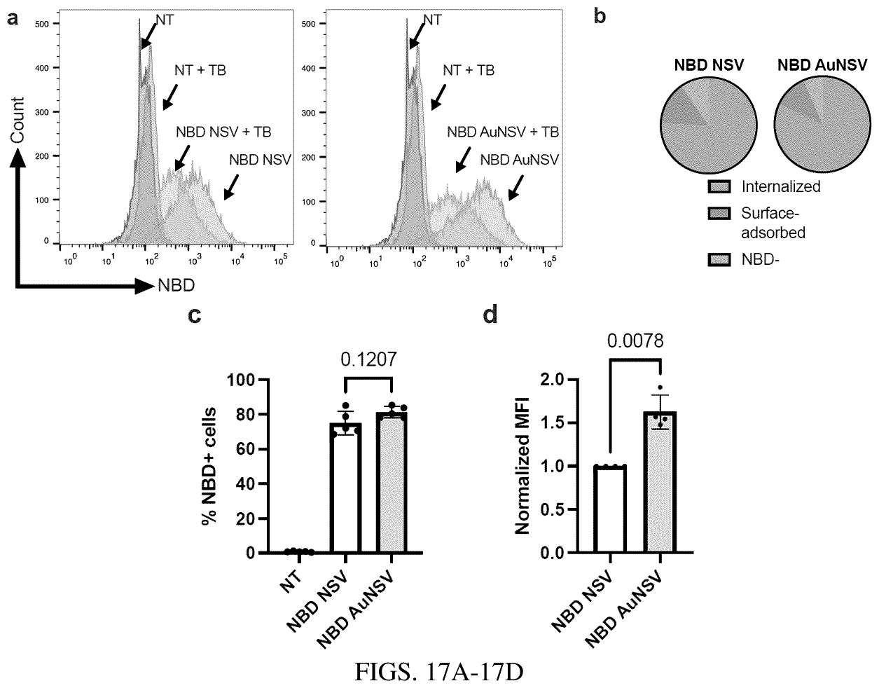

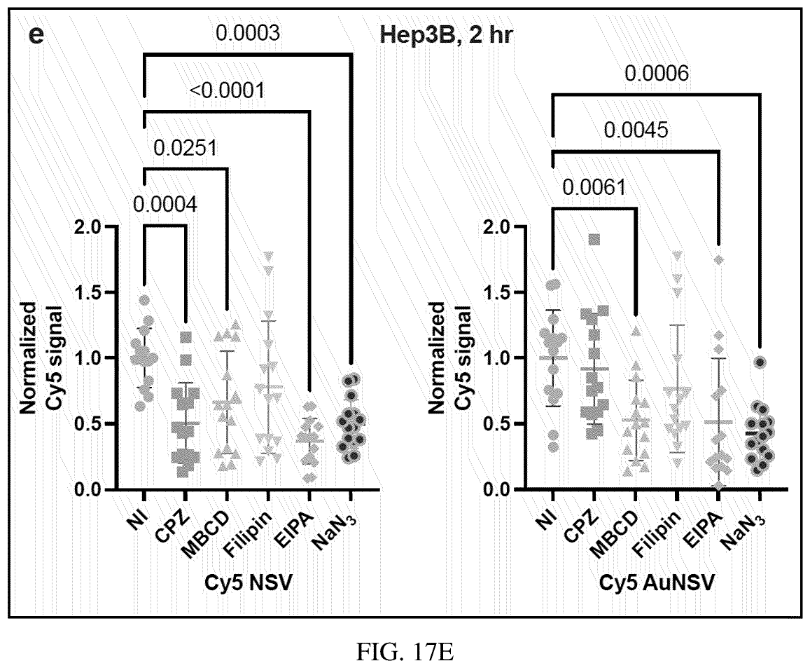

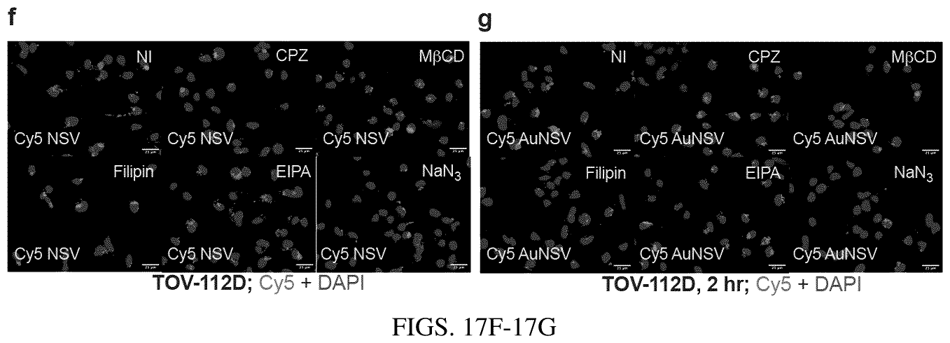

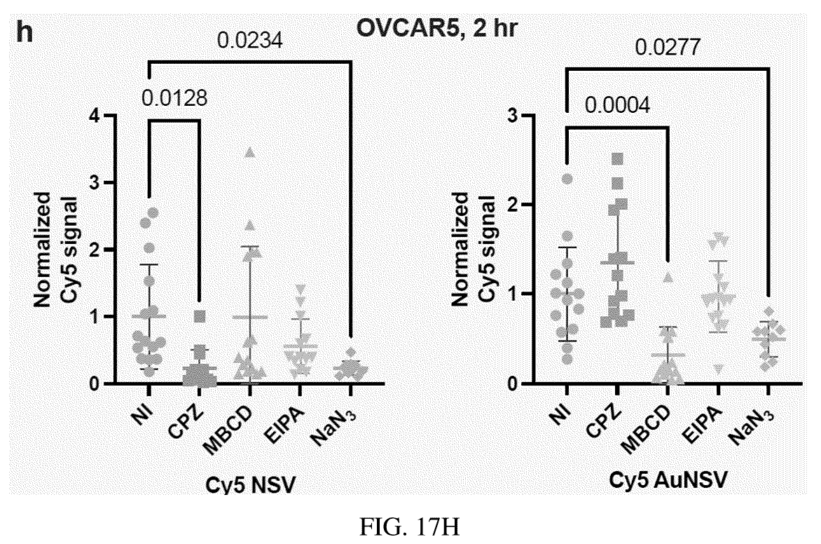

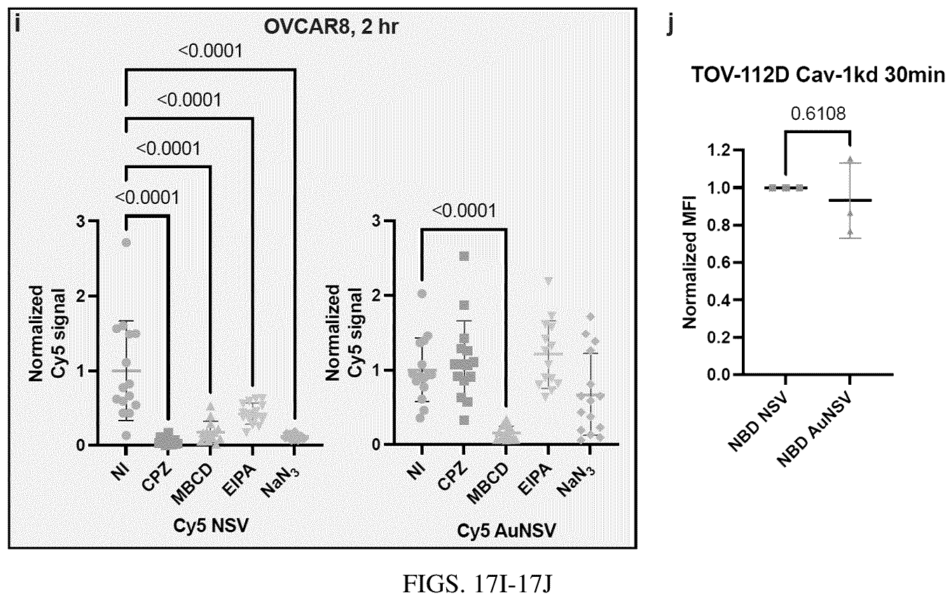

Based on this observation, we performed uptake inhibition studies at 30 minutes and 2 hours with Cy5-labeled NSVs or AuNSVs to identify the pathway by which nanoparticles entered the cells. Chlorpromazine (CPZ) was used to inhibit clathrin-mediated endocytosis (CME), methyl-β-cyclodextrin (MβCD) and filipin were used to inhibit caveolae-mediated endocytosis (CvME), 5-(N-Ethyl-N-isopropyl)-Amiloride (EIPA) was used to inhibit macropinocytosis, and sodium azide (NaN₃) was utilized to inhibit all forms of active transport. At the 30-minute time point, both Cy5 NSV and Cy5 AuNSV endocytosis was significantly inhibited by CPZ, suggesting that both vectors enter the cell predominantly via CME as expected from prior studies (FIG. 16E) (Hossen et al., Science Advances 6, caba5379, (2020); Gilleron et al., Nature Biotechnology 31, 638-646, (2013)). After 2 hours of delivery, however, Cy5 NSV uptake was still significantly inhibited by CPZ pretreatment with some effect of EIPA whereas Cy5 AuNSV endocytosis was no longer inhibited by CPZ and was instead greatly reduced by pretreatment with MβCD and filipin (FIG. 16F). Importantly, this shift in uptake mechanism towards CvME was observed in several cancer cell lines previously observed to display enhanced mRNA delivery and translation with AuNSV vectors (FIG. 16G, FIG. 17A-H; I)). Taken together, this evidence indicates that inclusion of AuNPs in NSV vectors induces cancer cells to preferentially activate CvME after initial uptake by CME, resulting in significantly increased uptake of additional AuNSVs.

While chemical inhibitor studies present strong evidence for a shift in nanoparticle uptake, biological inhibition presents further substantiation for a AuNSV-mediated shift to CvME. We thus performed a stable knockdown of Cav-1 (a key structural protein in caveolae) in TOV-112D cells to create a model system to biologically inhibit CvME. When we treated TOV-112D wild type (wt) cells with Cy5 GFP NSVs and Cy5 GFP AuNSVs for 24 hours, we observed the expected increase in uptake for AuNSVs in comparison to controls. When we performed the same study with Cav1 knockdown (kd) cells, however, we observed no difference in uptake between the two treatments because the uptake of the Cy5 GFP AuNSVs had decreased to match that of the Cy5 GFP NSVs (FIG. 16H).

We next utilized the Cav-1kd cells in uptake inhibition studies performed after 2 hours of delivery. The differential vector uptake observed in the wt cells was lost in the Cav-1 kd cells, further supporting the mechanism of a CvME switch (FIG. 16I). At the same time, we observed a significant and parallel uptake dependence on CME and macropinocytosis for both vectors, thus confirming the critical role of Cav-1 on the augmented uptake of AuNSVs (FIG. 16J). As further support, we studied the uptake of NSVs and AuNSVs in TOV-112D Cav-1 kd cells at 30 minutes and observed no differences between the two treatments (FIG. 17J). We can thus conclude that the inclusion of AuNPs within the AuNSV vector results in preferential activation of CvME between 30 minutes and 2 hours of treatment, resulting in greatly increased uptake of additional AuNSVs.

### AuNSVs Promote Endosomal Escape by Inhibiting Rab7-Mediated Endo-Lysosomal Fusion

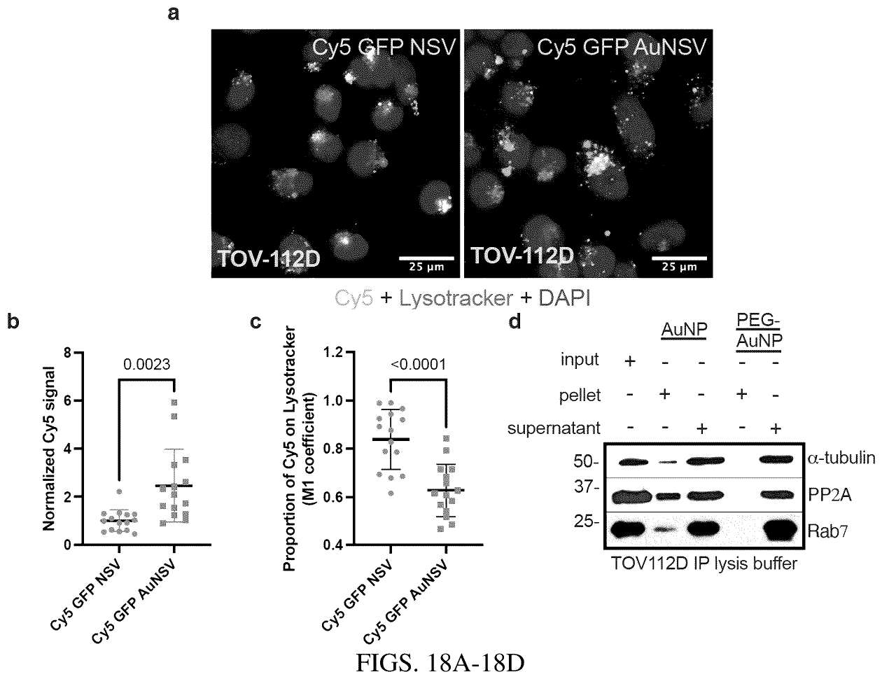

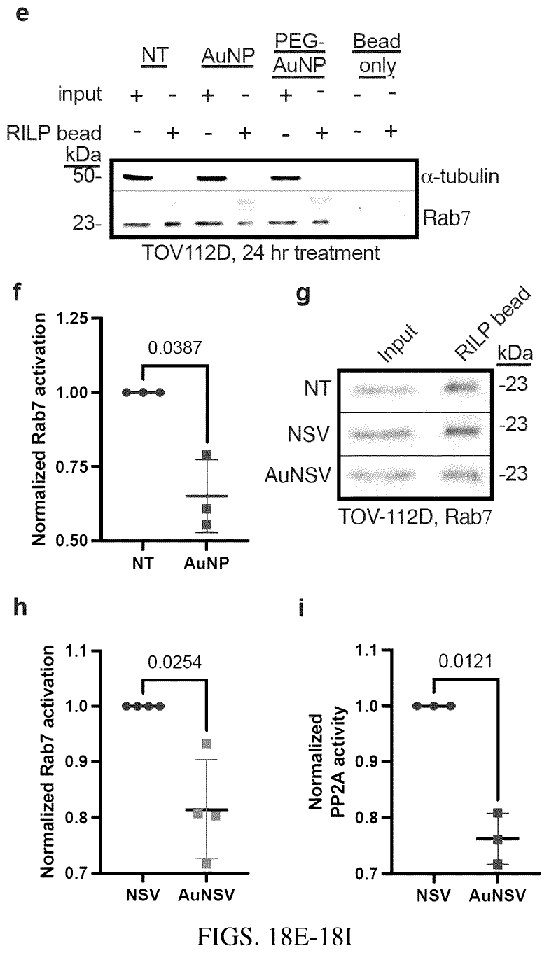

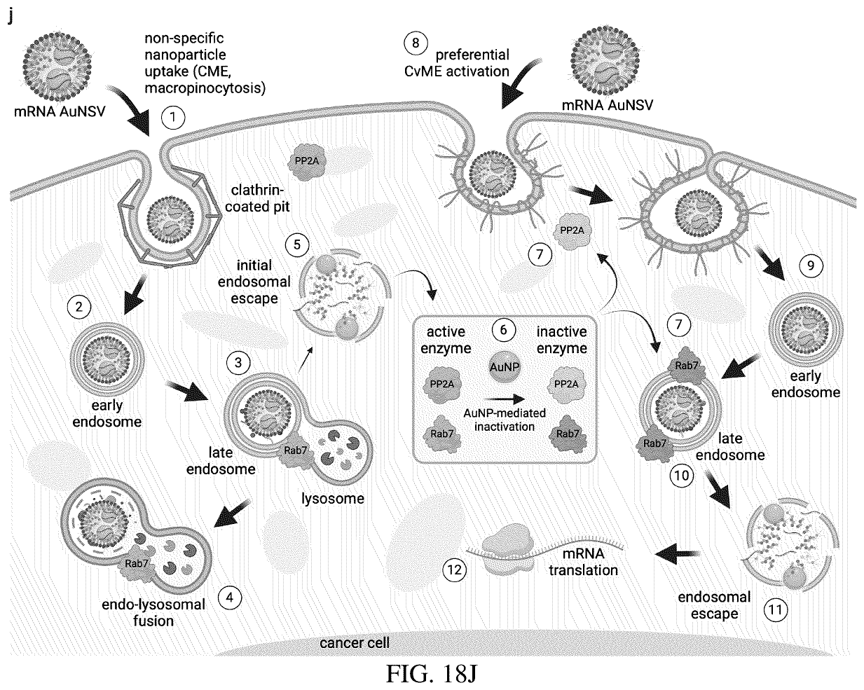

While increased uptake rate and a shift in uptake pathway may be partially responsible for the observed augmentation in delivery efficacy, we hypothesized that there must be one or more additional mechanisms by which AuNPs promote mRNA translation. When a delivery vector is taken up into a cell, it is normally processed sequentially into early and late endosomes. If the cargo does not escape the endosomal system, it is ultimately trafficked to the lysosome for enzymatic cleavage and degradation. Consequently, we observed trafficking of Cy5 GFP NSVs and Cy5 GFP AuNSVs in relation to lysosomes stained with Lysotracker™ (FIG. 18A). We observed not only greater uptake of Cy5 GFP AuNSVs (FIG. 18B), but also reduced colocalization of AuNSVs with Lysotracker™ (FIG. 18C), which suggests both trafficking and uptake components to the mechanism of AuNSV uptake. This revealed to us that AuNPs must act prior to the lysosome to disrupt cellular trafficking and promote endosomal escape.

From prior studies we know that 20 nm AuNPs can adsorb and inactivate PP2A, a phosphatase that mediates caveolae recycling and lipid raft fluidity, and small GTPases such as KRAS (Elechalawar et al., ACS Nano 17, 9326-9337 (2023); Hossen et al., Science Advances 6, caba5379 (2020)). Rab7 is a small GTPase localized to the late endosome and is the key regulator of endo-lysosomal fusion. We therefore hypothesized that both PP2A and Rab7 would be found in the AuNP protein corona and that treatment of cells with AuNSVs would inhibit the activity of these enzymes. As suspected, we identified both PP2A and Rab7 in the protein corona of bare AuNPs but not in the protein corona of AuNPs coated with poly(ethylene glycol) (PEG) (FIG. 18D).

We next performed pull-down assays using beads coated in Rab-interacting lysosomal protein (RILP), a protein with discriminatory binding to the active form of Rab7. TOV-112D cells treated with AuNPs displayed significantly reduced Rab7 activation in comparison to untreated cells, an effect that was rescued by PEGylation of the AuNPs (FIG. 18E-F). Similarly, AuNSV treatment significantly reduced Rab7 activation and PP2A activity in comparison to NSV controls (FIG. 18G-I). Consequently, we have identified two enzymes that are critical to the intracellular trafficking network that AuNPs bind to and disable with the result of enhanced vector uptake and endosomal escape. A diagram describing the mechanism of AuNSV-mediated mRNA delivery is provided in FIG. 18J.

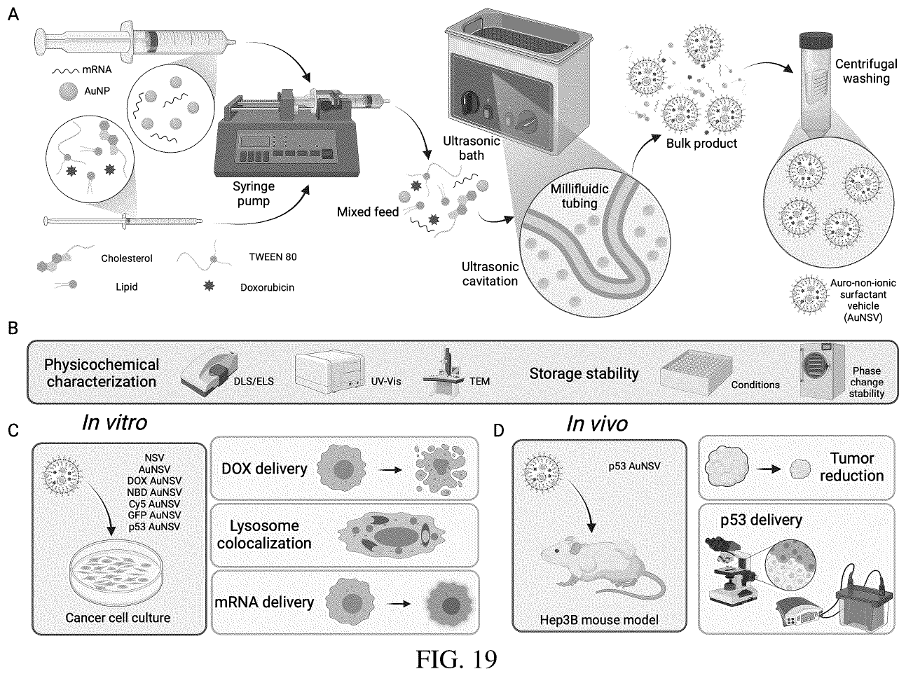

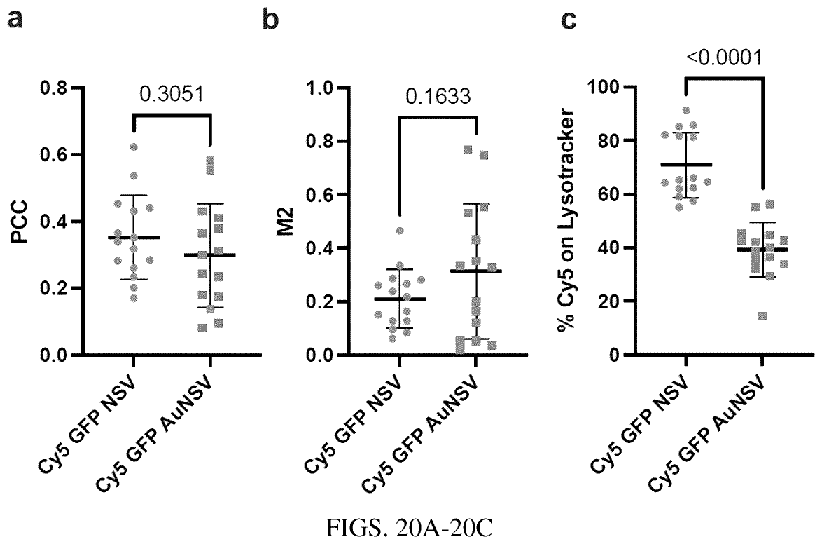

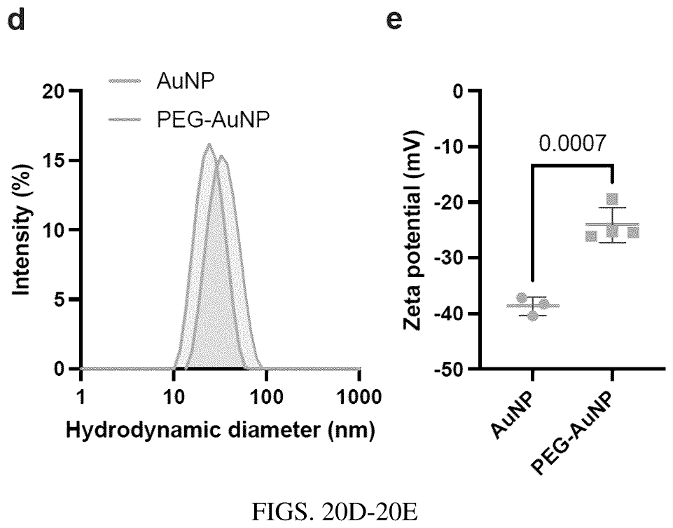

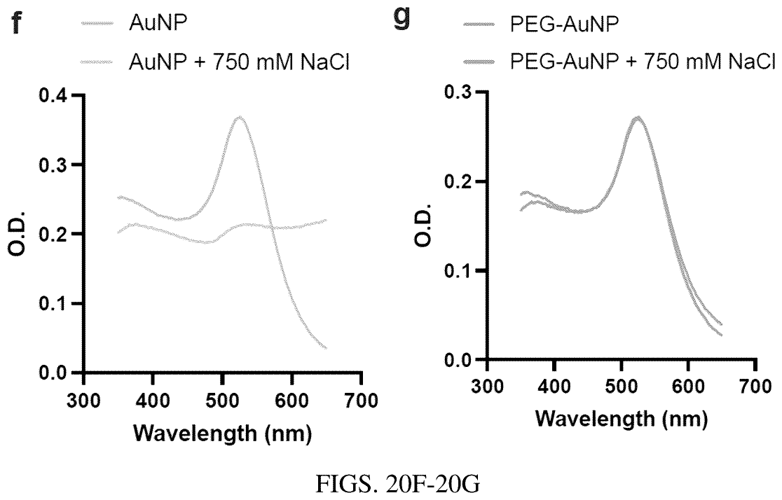

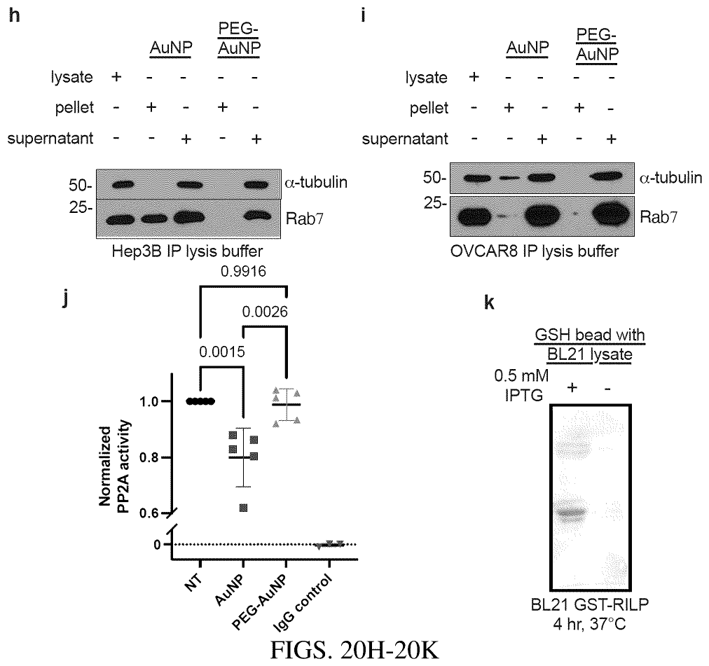

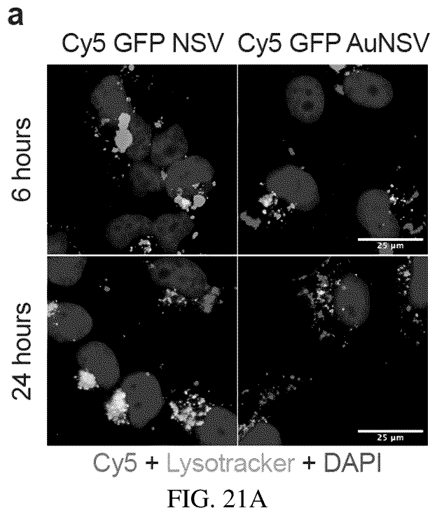

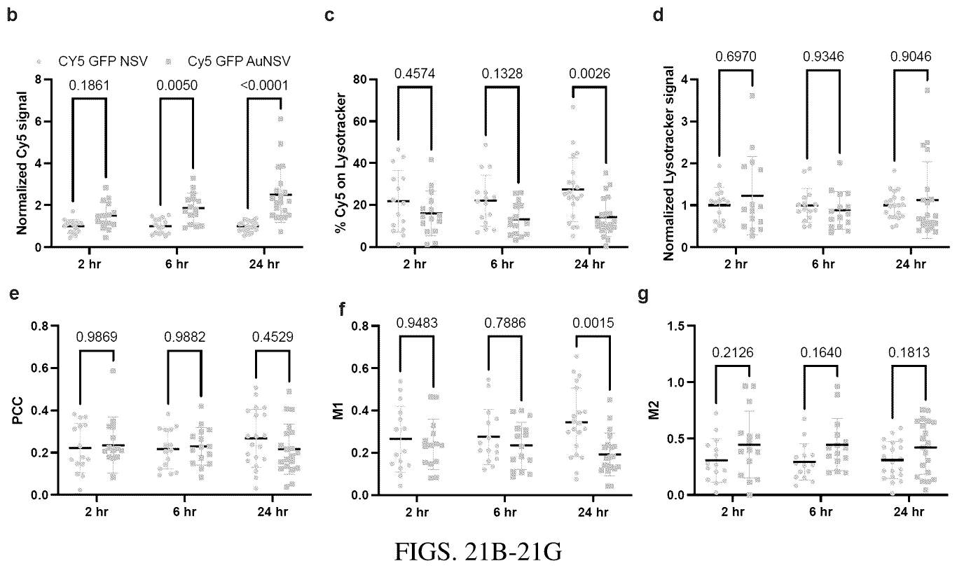

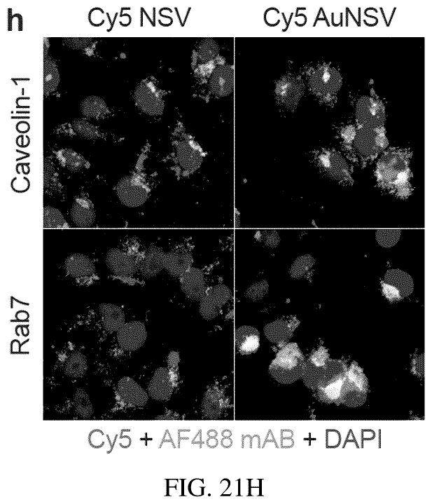

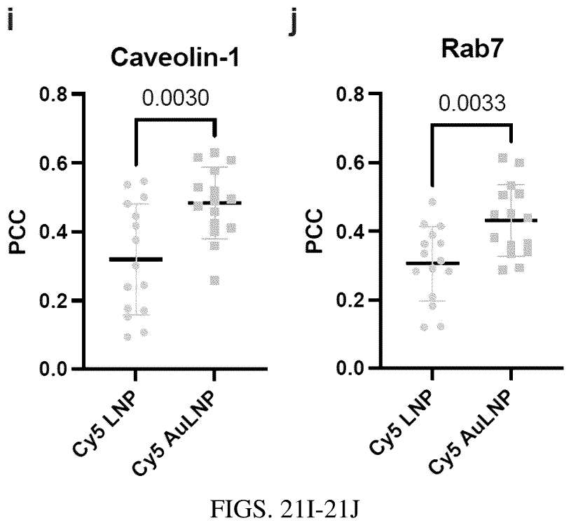

In the present disclosure, novel AuNSV nanomaterials were synthesized using a novel millifluidic synthesis process (FIG. 19). A library of NSVs and AuNSVs were created using differing ratios of lipid components and characterized for size, PDI, zeta potential, and encapsulation efficiency. AuNSVs are quite stable both in solution and through phase changes with the addition of 5% sucrose as a cryoprotectant. AuNSVs successfully encapsulate both chemotherapeutics and biologics at high loading capacities and can be modified with PEGylated or fluorescently-tagged lipids. AuNSVs enhance mRNA delivery in comparison to NSVs through inhibition of Rab7 and PP2A, with the result of increased AuNSV uptake and enhanced endosomal escape. Finally, AuNSV delivery of therapeutic p53 mRNA in hepatocellular carcinoma and ovarian cancer model systems showed significant improvements in comparison to p53 NSV therapy both in vitro and in vivo.

The investigations in the development of this nanomaterial were undertaken with the goal of clinical translation. Each of the components utilized were selected for favorable biomedical properties, stability, and prior reports of therapeutic efficacy. Additionally, the millifluidic process that was established to synthesize the AuNSVs was created with an emphasis on simplicity in design, case of operation, and high throughput to allow for facile scale up/out. The benchtop model in this study produced AuNSVs at 5.1 mL/min and can be readily scaled out for increased production. Importantly, the millifluidic tubing can be purchased off-the-shelf, allowing for reduced down time for system maintenance and precluding the need for precisely-manufactured channels frequently seen in microfluidic synthesis processes. Additionally, the increased diameter of the millifluidic tube in comparison to microfluidic counterparts leads to greatly reduced pressure drop and thus lower operating pressures along with reduced fouling.

The combination of millifluidic tubing and ultrasonic cavitation has been previously shown to promote self-assembly of polymer-protein nanoparticles (Seaberg et al., Eur J Pharm Biopharm 154, 127-135 (2020)), but to our knowledge no one has utilized a millifluidic process to produce self-assembled lipid-based nanosystems using ultrasonication to promote mixing in laminar phase flow. Ultrasound produces acoustic cavitation bubbles in an adiabatic manner on a scale similar to that of the millifluidic tubing (Rooze et al., Ultrasonics Sonochemistry 20, 1-11 (2013)). In a mechanism similar to that used in ultrasonic cleaning applications, cavitation bubbles form on the wall and subsequently collapse, creating jets that disrupt the laminar flow regime within the millifluidic channel and promote mixing between aqueous and lipid streams (Rooze et al., Ultrasonics Sonochemistry 20, 1-11 (2013)). As ultrasonic cavitation can put notable stress on biomolecules exposed to such conditions, we utilized materials with high stability as well as a high flow rate to mitigate any potentially detrimental effects of ultrasonic cavitation on our reagents.

One notable challenge in translating lipid nanoparticle therapies to the clinic, particularly those delivering mRNA, is the storage conditions and shelf stability of the vector and loaded biologics. Prior studies have reported that lyophilization of lipid-based nanosystems can lead to both aggregation and leakage of encapsulated therapeutics (Franzé et al., Pharmaceutics 10 (2018); Suzuki et al., Molecular Therapy-Nucleic Acids 30, 226-240 (2022)). When stabilized with 5% sucrose, AuNSVs displayed remarkable stability through both freeze/thaw cycles and lyophilization/rehydration. The combination of stable lipid components with a sucrose excipient allowed for consistent maintenance of physicochemical properties through phase changes, with no significant differences detected after 4 cycles of freezing at 20° C. and thawing. This is consistent with prior results showing that maintenance of mRNA efficacy for 3 months when LNPs are lyophilized or frozen with 5% sucrose or trehalose (Zhao et al., Bioactive Materials 5, 358-363 (2020)).

We showed that AuNSVs are capable of co-encapsulation of chemotherapeutics and AuNPs. By utilizing a 25:1 lipid:AuNP ratio we observed a DOX loading capacities similar to that of DOX NSVs. This is a notable result, because prior studies have reported significantly reduced DOX encapsulation with co-loading of AuNPs (Karimi et al., IET Nanobiotechnol 12, 846-849 (2018)). Both DOX NSVs and DOX AuNSVs delivered DOX to TOV-112D cells, with DOX AuNSV treatment showing greater significant differences between vehicle controls and treatment groups at low DOX concentrations. Previous studies have shown that combination therapy of DOX and AuNPs can promote therapeutic efficacy (Du et al., Drug Delivery. Bioconjugate Chemistry 29, 420-430 (2018)), counteract DOX resistance (Wang et al., ACS Nano 5, 3679-3692 (2011)), promote synergistic photothermal therapy (Xing et al., Nanotechnology 29, 405101 (2018)), and allow theranostic delivery and imaging (Manivasagan et al., International Journal of Biological Macromolecules 91, 578-588 (2016)). These may apply to DOX AuNSVs as well; however, the model system we utilized was not designed to answer these specific questions, but rather to provide evidence that AuNSVs can efficiently encapsulate and deliver chemotherapeutics to a model system.

DOX readily diffuses across membranes to localize within the nucleolus and inhibit DNA replication, and as such in vitro studies of nanoparticle-mediated delivery may not show enhanced efficacy over free DOX (Farhane et al., Analytical and Bioanalytical Chemistry 409, 1333-1346 (2017)). That there were greater differences in cell viability between DOX AuNSVs and controls in comparison to other treatments suggests that AuNPs enhance delivery through alteration of cellular uptake and trafficking, allowing DOX to enter the cell with greater efficiency than by diffusion alone. Additionally, the concentration of AuNPs utilized in this study was far below the therapeutic threshold, further supporting the idea that AuNPs are promoting DOX therapy rather than enacting a therapeutic effect on their own.

Along with chemotherapeutics, we developed AuNSVs to study the effects of 20 nm AuNPs on delivery mRNA to ovarian and liver cancers. We observed markedly improved delivery and therapeutic efficacy in vitro and in vivo when delivering reporter and therapeutic mRNA via AuNSV in comparison to NSVs. This difference was identified to be the result of a “Trojan horse” mechanism through which AuNPs inactivate mediators of lipid raft stability and endosomal trafficking, resulting in significantly increased vector uptake and endosomal escape. As such, we have made two major advances: 1) nanoengineer an AuNP-mRNA hybrid therapeutic with translatable characteristics, and 2) improve mRNA delivery to cancer cells using a two-step macromolecular mechanism.

In the course of this study, we first constructed a millifluidic synthesis apparatus to create stable mRNA-loaded AuNSVs that retain their physicochemical properties and efficacy through extended storage periods and freeze/thaw cycles, all of which are favorable characteristics for translatable therapeutics. Additionally, we studied several cancer cell lines and observed dependably enhanced levels of protein translation with AuNSV delivery. Finally, we showed that AuNPs not only increase the rate of AuNSV uptake, but they also prevent lysosomal degradation of these additional AuNSVs through inhibition of endo-lysosomal fusion. As a further benefit, our system is self-governing and does not require application of external stimuli to activate its functionality.

The multifactorial effects of AuNPs on mRNA delivery occasions a multifactorial explanation. AuNPs are known to exert self-therapeutic effects and improve delivery characteristics in the context of cancer therapy (Hossen et al., Science Advances 6, caba5379 (2020); Seaberg et al., Mater Today (Kidlington) 62, 190-224 (2023)). In this paper, we show that AuNPs activate a biological switch to CvME and prevent lysosomal colocalization by binding and inhibiting Rab7 and PP2A, enzymes regulating endo-lysosomal fusion and caveolar stability/internalization respectively. This is consistent with protein corona theory, which states that nanoparticles exposed to biological systems adsorb macromolecules and alter their conformation and activity (Mahmoudi et al., Nature Reviews Materials 8, 422-438 (2023)). AuNPs are known to bind and inhibit diverse cancer-associated proteins including KRAS, MMP9, SMAD2/3, and ICAM-1 and disrupt cellular crosstalk within the tumor microenvironment (Seaberg et al., Mater Today (Kidlington) 62, 190-224 (2023)); it follows that selective protein adsorption can be harnessed to promote endosomal trafficking dysregulation within the intracellular nanoenvironment. Inhibition of endosomal maturation and lysosomal fusion has been linked to endosomal escape through increased transport time and delayed acidification (Castro et al., Sci Rep 11, 7845 (2021)), and Rab7 is known to be integral to endosomal escape strategies utilized by pathogen and engineered therapeutics alike (Miller et al., Nucleic Acid Ther 28, 86-96 (2018); Cain et al., mBio 14, c0322122 (2023)). Nanoparticle endosomal escape occurs in vesicles containing characteristics of early and late endosomes, and Rab7 inhibition prevents maturation beyond this stage (Gilleron et al., Nature Biotechnology 31, 638-646 (2013)). Rab7 inhibition may also be key to developing brain-penetrating therapeutics based on reports of tumor extracellular vesicles penetrating the blood brain barrier by depleting endothelial Rab7 expression (Morad et al., ACS Nano 13, 13853-13865 (2019)). By employing AuNPs with known physicochemical properties capable of inhibiting small GTPases like Rab7, we designed our AuNSV vector to interrupt vesicular trafficking in the proximal mRNA vector nanoenvironment, thus promoting endosomal escape and enhancing vector efficiency (Elechalawar et al., ACS Nano 17, 9326-9337 (2023)).

Nanoenvironmental dysregulation radically altered the mechanism by which mRNA vectors were endocytosed. Early studies with the phosphatase inhibitors increased caveolae internalization and mobilization, predominantly through PP2A inactivation (Botos et al., Micron 38, 313-320 (2007); Fernandez et al., Current medicinal chemistry 9, 229-262 (2002)). In this mechanism, inhibition of PP2A reduces PP2A-mediated inhibition of Src kinase, which in turn phosphorylates Cav-1 and promotes caveolae internalization (Kiss & Botos, J Cell Mol Med 13, 1228-1237 (2009)). PP2A has been further hypothesized to mediate internal trafficking similar to the Rab family of proteins, though its importance in these processes has been minimized more recently (Kiss & Botos, J Cell Mol Med 13, 1228-1237 (2009)). Some publications have linked CvME with alternative internal trafficking and evasion of lysosomes (Hossen et al., Science Advances 6, caba5379 (2020); Kiss & Botos, J Cell Mol Med 13, 1228-1237 (2009); Yang et al., Anal Chem 94, 7960-7969 (2022)), whereas others suggest that CvME utilizes the standard Rab-mediated trafficking vesicles after initial uptake (Kiss & Botos, J Cell Mol Med 13, 1228-1237 (2009)). To cloud the waters, more recent evidence has cast doubts upon caveolae as vesicles for uptake and instead emphasizes their role in signaling and stress protection (Parton, Annu Rev Cell Dev Biol 34, 111-136 (2018); Rennick et al., Nature Nanotechnology 16, 266-276 (2021)). In this study, we show that AuNSVs activate an intracellular switch; whereas initial uptake (<30 minutes) occurs primarily via CME, later uptake (>2 hours) is significantly inhibited by disruption/depletion of membrane cholesterol (the mechanism of our small molecule inhibitors) and by knockdown of Cav-1 (an essential caveolae protein) (Patel & Insel, Antioxid Redox Signal 11, 1357-1372 (2009)). We also observed that AuNSVs inhibit PP2A activity, a mechanism that has been linked to enhanced caveolae internalization (Kiss & Botos, J Cell Mol Med 13, 1228-1237 (2009)). As such, we have evidence supporting activation of CvME, but we understand that caveolar uptake is an area of ongoing debate and do not presuppose to have the final word on the matter.

Several advantages are derived from identifying that Cav-1 as integral to the rapid uptake and delivery of AuNSVs. In the context of cancer therapy, Cav-1 has been reported as both an oncogenic molecule and as a tumor suppressor, and it has recently been identified as a molecular organizer due to the diverse pathways it plays a role in (Williams & Lisanti, American Journal of Physiology-Cell Physiology 288, C494-C506 (2005); Bhowmick et al., Biochim Biophys Acta Rev Cancer 1878, 189002 (2023); Díaz et al., Cell Death & Disease 11, 648 (2020); Chen & Che, Oncol Lett 8, 1409-1421 (2014)). Importantly, Cav-1 is differentially expressed in the cancer cells and tumor stromal cells, with poor prognosis corresponding to a marked increase of Cav-1 in cancer cells while decreased Cav-1 in the surrounding stromal cells (Chen & Che, Oncol Lett 8, 1409-1421 (2014); Qian et al., Cancer science 102, 1590-1596 (2011)). These data suggest that we may purposely activate vector uptake into cancer cells over adjacent fibroblasts and macrophages, though additional work will be required to elucidate these effects.

All told, we have described a “Trojan horse” delivery mechanism in which an initial AuNSV is endocytosed and subsequently inactivates two proteins: Rab7, which prevents lysosomal degradation and promotes endosomal escape, and PP2A, resulting in preferential activation of CvME. Whereas other “Trojan horse” delivery systems focus selectively on endosomal escape or disguising some therapeutic cargo (Xue et al., Nature Communications 9, 3653 (2018); Pardridge, Trends Mol Med 29, 343-353 (2023); Hsiao et al., Environmental Science & Technology 49, 3813-3821 (2015); Wu et al., Small 16, 2003757, (2020)), our design more accurately parallels the literary account. As the story goes, Odysseus and a small team of soldiers sneak inside the city of Troy within a wooden horse before breaking out and opening the main gates to allow the rest of the army to rapidly enter. Similarly, AuNSVs function by both promoting endosomal escape (breaking out of the horse) and by massively increasing the rate of nanoparticle uptake (opening the city gates). While each of these functions is individually beneficial, the combination of them is a dramatic step forward for nanotherapeutic design.

The results of this study open several areas of exploration in nanomedicine. To this point, most investigations have sought to increase delivery efficacy by optimizing lipid component selection. We, however, have shown that disruption of the endosomal nanoenvironment through inhibition of mediator proteins can significantly enhance delivery characteristics while simultaneously allowing use of low-cost, high-stability lipid components. From this starting point we may further understand how self-therapeutic nanomaterials and small molecules may be utilized to promote vector uptake and endosomal escape through dysregulation of endosomal trafficking. We plan to expand our studies to understand the differential effects of AuNPs on the uptake of lipid nanosystems in non-cancerous cells and for delivery of diverse therapeutics. We propose that this hybrid nanosystem and its novel mechanism of action could form the basis for a wide array of next-generation therapeutics.

### Millifluidic NSV Synthesis Apparatus

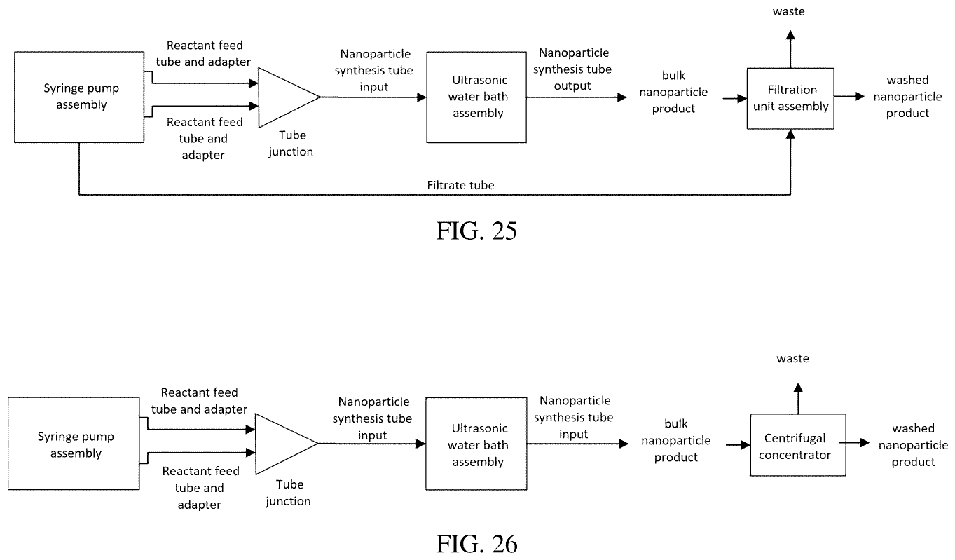

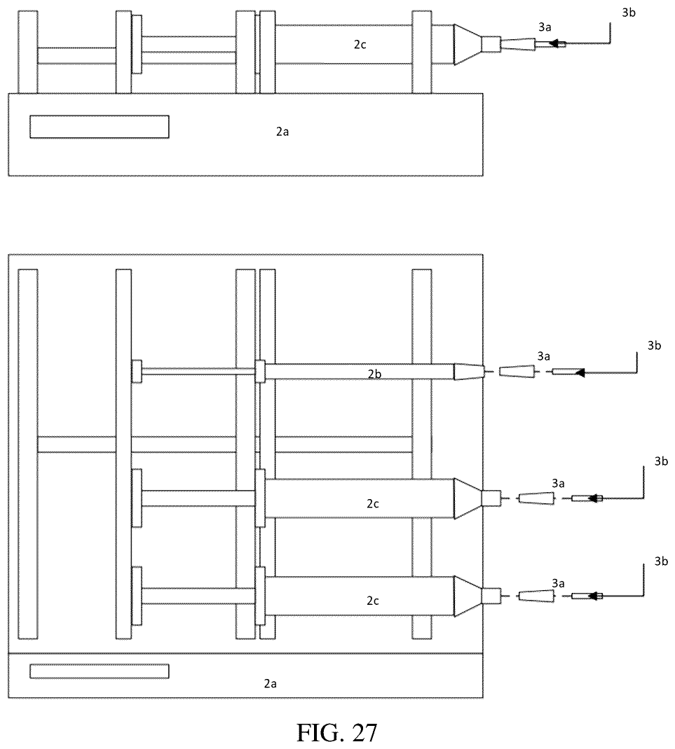

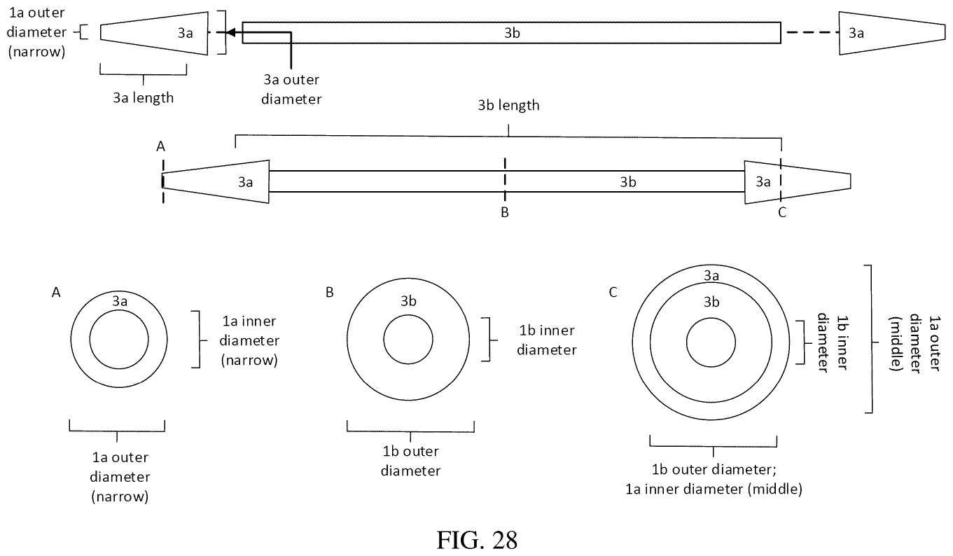

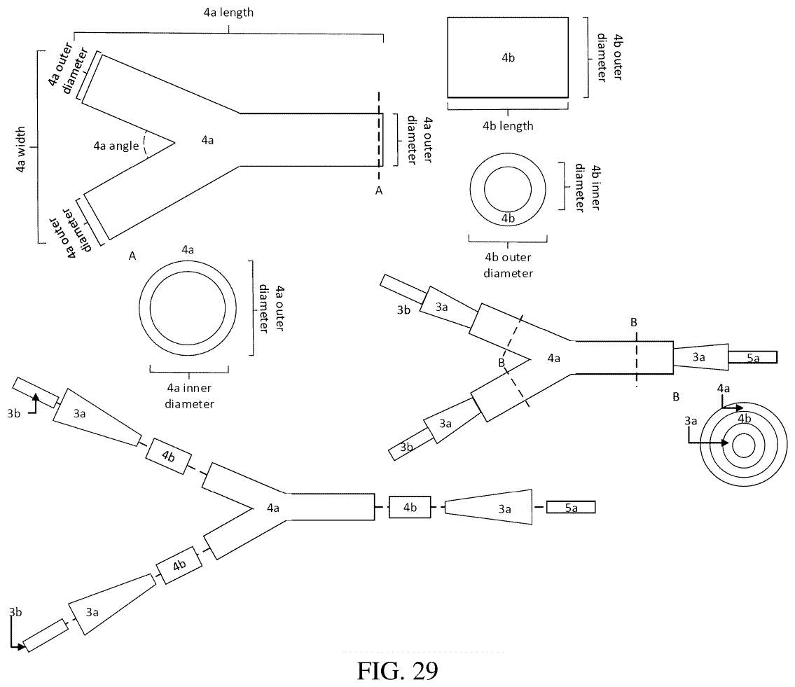

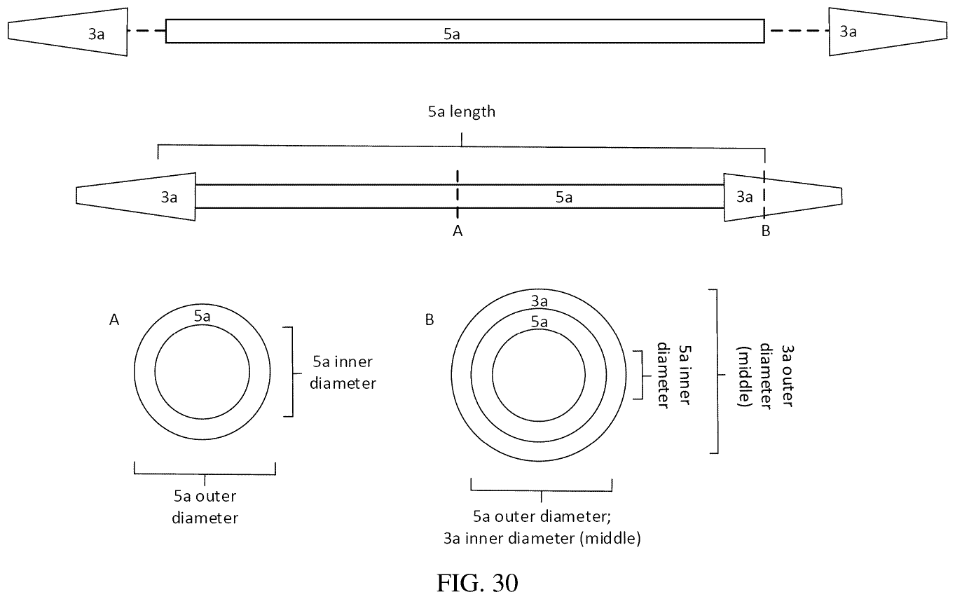

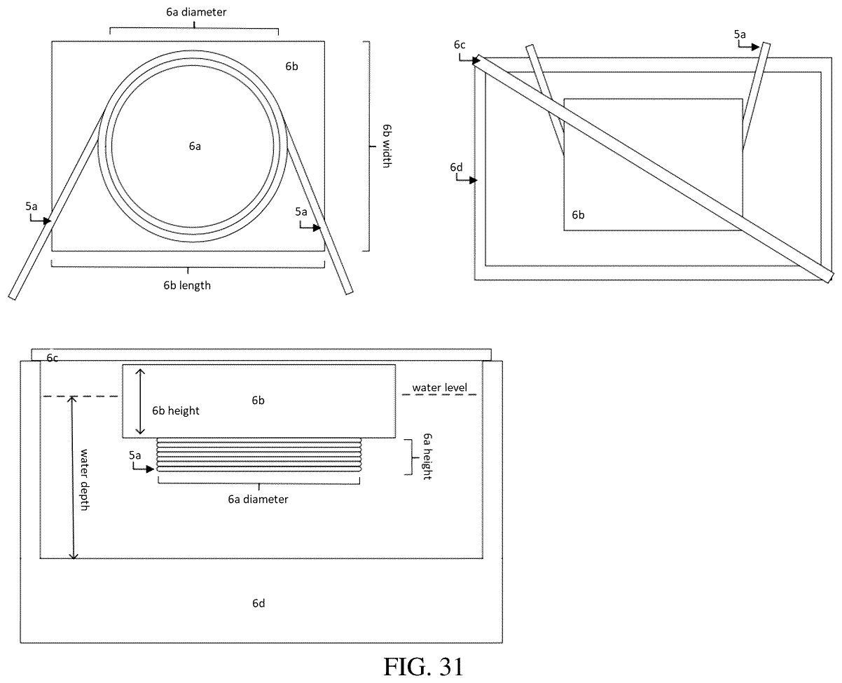

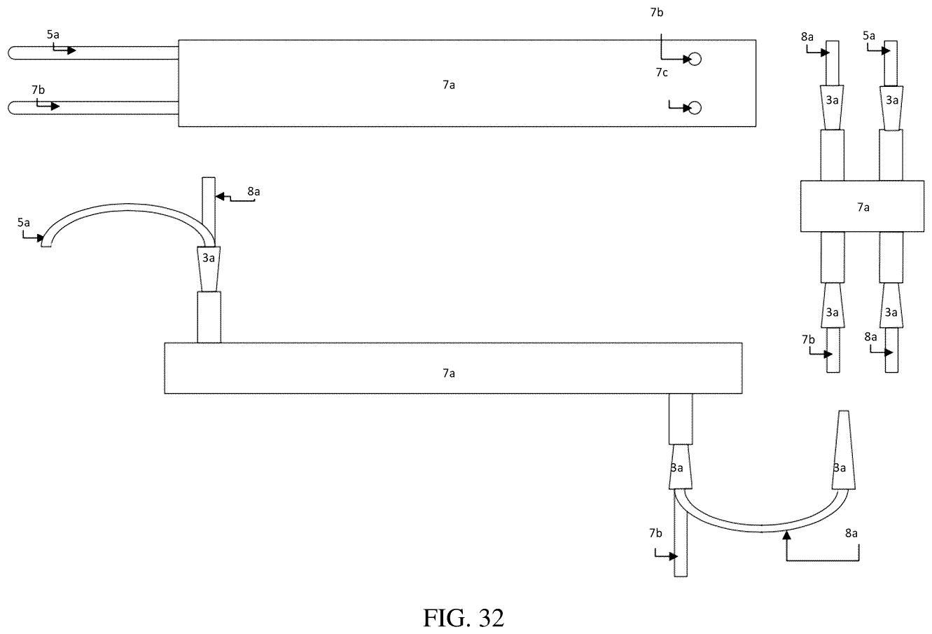

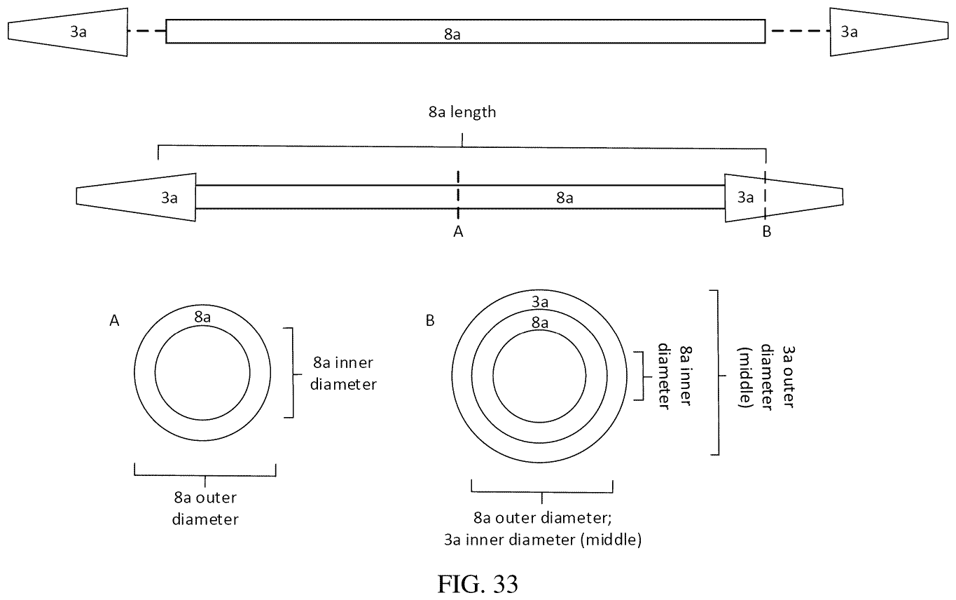

FIGS. 25 and 26 are schematics of non-limiting embodiments of a nanoparticle synthesis apparatus that may be used to synthesize the nanoparticles of the present disclosure. The embodiment of FIG. 25 utilizes a membrane filtration system to separate and purify the NPs. The embodiment of FIG. 26 utilizes a centrifugal concentrator washing system to separate and purify the NPs. FIGS. 27-33 show various subsystems and components of the nanoparticle synthesis apparatus of FIGS. 25-26. The following are non-limiting examples of various materials and devices that can be used to construct the various systems and components of FIGS. 27-33.

### Example 2

In at least certain embodiments, the apparatus for constructing the AuNSVs of the present disclosure comprises the following components constructed according to the following parameters:
2a: syringe pump; capable of using 1-100 mL syringes; flow rate: 10 μL/min-100 mL/min; 2-20 syringe capacity;
2b: syringe; polypropylene, high-density polyethylene, other rigid chemically-resistant polymer, glass; volume: 1-100 mL;
2c: syringe; polypropylene, high-density polyethylene, other rigid chemically-resistant polymer, glass; volume: 1-100 mL;
3a: tube adapter; polypropylene or other rigid chemically-resistant polymer such as high-density polyethylene; length: 2-4 cm; inner diameter (narrow): 0.4-0.7 mm; outer diameter (narrow): 0.6-1.2 mm; inner diameter (middle): 1.5-2.8 mm; outer diameter (middle): 1.7-3.0 mm; inner diameter (wide): 1.5-4.8 mm; outer diameter (wide): 1.7-5.0 mm;
3b: reactant feed tube; polytetrafluoroethylene (PTFE), Kynar® PVDF, Halar® ECTFE, PFA, FEP, other flexible chemically-resistant fluoropolymer, silicone, silicone rubber, TPE, or other flexible inert material; length: 2-20 cm; inner diameter: 0.1-1.0 mm; outer diameter: 0.5-3.0 mm;
4a: Y-junction; polypropylene or other rigid chemically-resistant polymer such as high-density polyethylene; length: 1-4 cm; width: 1-4 cm; angle: 10-180 degrees; inner diameter: 1.0-5.0 mm; outer diameter: 1.2-5.4 mm;
4b: bushing; silicone rubber, silicone, rubber, or other flexible rubberized material; length: 2-10 mm; inner diameter: 0.1-4.0 mm; outer diameter: 0.5-5.0 mm;
5a: nanoparticle synthesis tube; polytetrafluoroethylene (PTFE), Kynar® PVDF, Halar® ECTFE, PFA, FEP, other flexible chemically-resistant fluoropolymer, silicone, silicone rubber, TPE, or other flexible inert material; length: 50-1,000 cm; inner diameter: 0.1-3.0 mm; outer diameter: 0.5-3.5 mm;
6a: synthesis tube winding coil; polypropylene or other rigid polymer such as high-density polyethylene; diameter: 5-15 cm; height: 1-4 cm;
6b: stabilizing float; styrofoam or other buoyant material; length: 5-20 cm; width: 5-20 cm; height: 2-10 cm;
6c: float brace; polypropylene or other rigid polymer; length: 20-40 cm; width: 0.5-5 cm; height: 0.5-5 cm;
6d: ultrasonic water bath; 25-5000 W; 50/60 hz; water depth: 5-25 cm; volume: 0.5-10 L;
7a: membrane filtration system; filtration cutoff 10 kDa-300 kDa;
7b: product outlet tube; polytetrafluoroethylene (PTFE), Kynar® PVDF, Halar® ECTFE, PFA, FEP, other flexible chemically-resistant fluoropolymer, silicone, silicone rubber, TPE, or other flexible inert material; length: 5-200 cm; inner diameter: 0.5-13 mm; outer diameter: 0.7-15 mm; and
8a: filtrate tube; polytetrafluoroethylene (PTFE), Kynar® PVDF, Halar® ECTFE, PFA, FEP, other flexible chemically-resistant fluoropolymer, silicone, silicone rubber, TPE, or other flexible inert material; length: 5-200 cm; inner diameter: 0.5-13 mm; outer diameter: 0.7-15 mm.

### Example 3

In at least certain embodiments, the AuNSVs comprise the following components, and are made using an apparatus constructed according to the following parameters:
Functional lipid: 0.01-10 mg/mL; 0-80 wt %; long-chained fatty acids such as DDAB, DOTAP, DOPE, DSPE, EPC, stearic acid, stearyl alcohol, DOTMA, DODMA, DOBAQ, DAP, DOSPA;
Structural lipid: 0.01-10 mg/mL; 0-80 wt %; sterols, eg., cholesterol, DC-cholesterol;
Surfactant: 0.01-1000 mg/mL; 0.5-50 wt %; polysorbate 80 (Tween 80), polysorbate 20 (Tween 20);
Solvent: e.g., ethanol, dimethyl sulfoxide, acetone;
Polymer-lipid conjugate: Chol-PEG600, DSPE-PEG (n)(x), where n=500-5000 and x=maleimide, amine, folate, carboxy NHS, azide, carboxylic acid, biotin, PDP, succinyl, etc.;
Fluorescently labeled lipid: Cy5-PE, NBD-PE, Rhod-PE, Cy7-PE, NBD-PS, fluorescein-PE, AF488-PE, AF546-PE, DiR;
Hydrophobic cargo: doxorubicin, calcein, other hydrophobic therapeutics;
Aqueous buffer: sodium acetate, sodium citrate, PBS, etc.;
Inorganic nanoparticle: 1:1-1000:1 lipid:NP wt:wt; gold nanoparticle, silver nanoparticle, iron oxide nanoparticle, cuprous oxide nanoparticle, mesoporous silica nanoparticle etc.;
Hydrophilic cargo: 1:1-1000:1 lipid:NP wt:wt; mRNA, siRNA, CRISPR/Cas9 system, cisplatin, crystal violet, BSA, DQ-BSA, other hydrophilic therapeutics, adjuvants, or molecules;
Ultrasonic bath: 100-3500W, 50/60 hz, 0° C.-37° C.; and
Flow characteristics: 1:1-100:1 aqueous:lipid vol/time:vol/time; 50 μL/min-50 mL/min total.

### Example 4

In at least certain embodiments, the AuNSVs comprise the following components, and are made using an apparatus constructed according to the following parameters shown in Table 1 and Table 2:

TABLE 1
Exemplary operating parameter values

| Design | | | | |
|---|---|---|---|---|
| parameter | Units | Broad range | Medial range | Narrow range |
| Functional lipid | mg/mL | 0.01-10 | 0.1-10 | 1-3.5-6 |
| concentration | | | | |
| Functional lipid | wt % | 0-80 | 10-50 | 25-35-40 |
| wt % | | | | |
| Structural lipid | mg/mL | 0.01-10 | 0.1-10 | 1-4-7 |
| concentration | | | | |
| Structural lipid | wt % | 0-80 | 10-75 | 25-40-50 |
| wt % | | | | |
| Surfactant | mg/mL | 0.01-1000 | 0.1-10 | 1-2.5-5 |
| concentration | | | | |
| Surfactant wt % | wt % | 0.5-50 | 5.0-35 | 15-25-30 |
| Polymer-lipid | mg/mL | 0.001-10 | 0.01-2 | 0.1-0.5-1 |
| conjugate | | | | |
| concentration | | | | |
| Polymer-lipid | wt % | 0.01-10 | 0.05-2 | 0.05-0.1-1 |
| conjugate wt % | | | | |
| Fluorescently | mg/mL | 0.001-10 | 0.01-2 | 0.1-0.5-1 |
| labeled-lipid | | | | |
| conjugate | | | | |
| concentration | | | | |
| Fluorescently | wt % | 0.01-10 | 0.05-2 | 0.05-0.1-1 |
| labeled-lipid | | | | |
| conjugate wt % | | | | |
| Hydrophobic | mg/mL | 0.001-10 | 0.1-5 | 0.5-1.5-2 |
| cargo | | | | |
| concentration | | | | |
| Hydrophobic | N/A | 1:1-10,000:1 | 1:1-25:1 | 1:1-2.5:1-5:1 |
| cargo lipid:cargo | | | | |
| wt ratio | | | | |
| Aqueous buffer | pH | 0-10 | 2-6 | 3-4-5 |
| pH | | | | |
| Inorganic | μg/mL | 0.01-10,000 | 1-1000 | 10-140-200 |
| nanoparticle | | | | |
| concentration | | | | |
| Inorganic | N/A | 1:1-250:1 | 1:1-25:1 | 1:1-2.5:1-5:1 |
| nanoparticle | | | | |
| lipid:NP wt ratio | | | | |
| Hydrophilic | μg/mL | 0.1-100 | 1-50 | 10-26-40 |
| cargo | | | | |
| concentration | | | | |
| Hydrophilic | N/A | 1:1-1000:1 | 1:1-100:1 | 1:1-25:1-50:1 |
| cargo lipid:cargo | | | | |
| wt ratio | | | | |
| Aqueous:Lipid | N/A | 1:1-100:1 | 3:1-25:1 | 5:1-9:1-15:1 |
| stream flow ratio | | | | |
| Combined | mL/min | 0.1-100 | 1-10 | 3-5-7 |
| reactant stream | | | | |
| flow rate | | | | |
| Reactant stream | N/A | 1-2500 | 5-1000 | 50-90-150 |
| Reynolds | | | | |
| number | | | | |

TABLE 2
Exemplary apparatus parameter values

| Apparatus | | | | |
|---|---|---|---|---|
| element | Unit | Broad range | Medial range | Narrow range |
| 2c Aqueous | mL | 0.5-100 | 1-25 | 5-10-20 |
| stream syringe | | | | |
| volume | | | | |
| 2b Lipid stream | mL | 0.5-100 | 1-20 | 1-5-10 |
| syringe volume | | | | |
| 3b Reactant feed | cm | 1-100 | 2-25 | 5-10-15 |
| tube length | | | | |
| 3b Reactant feed | mm | 0.1-3 | 0.25-1 | 0.5-0.3048-0.75 |
| tube inner | | | | |
| diameter | | | | |
| 3b Reactant feed | mm | 0.5-5 | 1-3.5 | 1-1.5875-2 |
| tube outer | | | | |
| diameter | | | | |
| 3a Tube adapter | cm | 0.5-5 | 1-4 | 2-2.5-3 |
| length | | | | |
| 3a Tube adapter | mm | 0.1-5 | 0.25-1.5 | 0.3-0.5-1 |
| inner diameter | | | | |
| (narrow) | | | | |
| 3a Tube adapter | mm | 1-10 | 1.5-5 | 2-3.8-4 |
| inner diameter | | | | |
| (wide) | | | | |
| 3a Tube adapter | mm | 0.1-3.0 | 0.5-1.5 | 0.75-0.9-1.2 |
| outer diameter | | | | |
| (narrow) | | | | |
| 3a Tube adapter | mm | 1-10 | 1.5-6.0 | 3-4.2-5 |
| outer diameter | | | | |
| (wide) | | | | |
| 4a Y-junction | cm | 1-10 | 2-4 | 2-2.5-3 |
| length | | | | |
| 4a Y-junction | mm | 1-15 | 2-7.5 | 2.5-3.175-4 |
| inner diameter | | | | |
| 4a Y-junction | mm | 1-15 | 2-7.5 | 2.5-3.5-5 |
| outer diameter | | | | |
| 4a Y-junction | cm | 0.5-5 | 1-2.5 | 1-1.25-2 |
| outer width | | | | |
| 4a Y-junction | ° | 10-180 | 30-120 | 50-60-80 |
| angle | | | | |
| 4b Bushing | mm | 1-50 | 2-15 | 4-8 |
| length | | | | |
| 4b Bushing inner | mm | 0.5-3.5 | 1-2.5 | 1-1.5875-2 |
| diameter | | | | |
| 4b Bushing outer | mm | 1-15 | 2-5 | 2.5-3.175-4 |
| diameter | | | | |
| 5a Nanoparticle | cm | 50-1000 | 100-500 | 200-260-300 |
| synthesis tube | | | | |
| length | | | | |
| 5a Nanoparticle | mm | 0.1-3.0 | 0.25-1.5 | 0.5-0.784-1 |
| synthesis tube | | | | |
| inner diameter | | | | |
| 5a Nanoparticle | mm | 0.5-3.5 | 1-2.5 | 1-1.5875-2 |
| synthesis tube | | | | |
| outer diameter | | | | |
| 6a Synthesis | cm | 1-100 | 5-25 | 7.5-10-15 |
| tube helical | | | | |
| winding coil | | | | |
| diameter | | | | |
| 6a Synthesis | cm | 0.5-10 | 1-5 | 1.5-2-3 |
| tube helical | | | | |
| winding coil | | | | |
| height | | | | |
| 6b stabilizing | cm | 2-50 | 5-20 | 7.5-10-15 |
| float length | | | | |
| 6b stabilizing | cm | 2-50 | 5-20 | 7.5-9-12 |
| float width | | | | |
| 6b stabilizing | cm | 1-15 | 2-10 | 2.5-3-5 |
| float height | | | | |
| 6c float brace | cm | 10-100 | 15-50 | 20-28-35 |
| length | | | | |
| 6c float brace | cm | 0.1-20 | 0.25-5 | 0.25-0.5-2 |
| width | | | | |
| 6c float brace | cm | 0.2-20 | 0.25-5 | 0.25-0.5-2 |
| height | | | | |
| 6d Ultrasonic | W | 50-5000 | 150-500 | 200-280-350 |
| bath power | | | | |
| 6d Ultrasonic | ° C. | 0-50 | 0-37 | 0-25 |
| bath operating | | | | |
| temperature | | | | |
| 7a Membrane | kDa | 5-1,000 | 50-500 | 200-300-400 |
| filtration system | | | | |
| cutoff | | | | |
| 7b Product outlet | cm | 0-1000 | 5-100 | 10-20-50 |
| tube length | | | | |
| 7b Product outlet | mm | 0.1-50 | 0.5-15 | 1-5-10 |
| tube inner | | | | |
| diameter | | | | |
| 7b Product outlet | mm | 0.1-50 | 0.7-15 | 2.5-8-12 |
| tube outer | | | | |
| diameter | | | | |
| 8a Filtrate tube | cm | 50-1000 | 5-500 | 100-260-350 |
| length | | | | |
| 8a Filtrate tube | mm | 0.1-50 | 0.25-13 | 0.5-0.784-5 |
| inner diameter | | | | |
| 8a Filtrate tube | mm | 0.5-50 | 0.7-15 | 1-1.5875-5 |
| outer diameter | | | | |

The apparatus and parameters can be scaled up in size for commercial levels of production. For example an apparatus can be constructed using a 1 cm diameter tube and a flow rate of 5 L/min. This results in a Reynold's number of approximately 6,760, which is a sufficiently turbulent flow to induce mixing, so that agitation with an ultrasonic water bath is not necessary for formation of the AuNSVs.

In one embodiment, to maintain laminar flow, the highest usable Reynold's number is approximately 2300, which enables a 5 L/min flow rate with a tube diameter of 3 cm. Under these conditions, ultrasonic mixing in a water bath is necessary. The syringe pump could be replaced with alternative pumping devices.

It will be understood from the foregoing description that various modifications and changes may be made in the various embodiments of the present disclosure without departing from their true spirit. The description provided herein is intended for purposes of illustration only and is not intended to be construed in a limiting sense, except where specifically indicated. Thus, while the present disclosure has been described herein in connection with certain non-limiting embodiments so that aspects thereof may be more fully understood and appreciated, it is not intended that the present disclosure be limited to these particular embodiments. On the contrary, it is intended that all alternatives, modifications, and equivalents are included within the scope of the present disclosure as defined herein. Thus, the examples described above, which include particular embodiments, will serve to illustrate the practice of the present disclosure, it being understood that the particulars shown are by way of example and for purposes of illustrative discussion of particular embodiments only and are presented in the cause of providing what is believed to be a useful and readily understood description of procedures as well as of the principles and conceptual aspects of the inventive concepts. Changes may be made in the formulation of the various components and compositions described herein, the methods described herein, or in the steps or the sequence of steps of the methods described herein, without departing from the spirit and scope of the present disclosure.

1. A method of encapsulating therapeutics, the method comprising the steps of:
providing a hydrophobic fluid stream comprising hydrophobic structural materials and hydrophobic cargo molecules;
providing an aqueous stream comprising hydrophilic cargo molecules in aqueous buffer and optionally comprising hydrophilic structural materials;
combining the hydrophobic fluid stream with the aqueous stream to form a mixed feed stream;
passing the mixed feed stream through a millifluidic tubing which is disposed in an ultrasonic bath at a temperature in a range from about 0° C. to about 37° C., wherein the cargo molecules and the structural molecules are combined within the millifluidic tubing to form encapsulated therapeutics; and
purifying the encapsulated therapeutics.

2. The method of claim 1, wherein the hydrophobic structural materials are lipids.

3. The method of claim 1, wherein the hydrophilic structural materials are polymers.

4. The method of claim 1, wherein the hydrophilic cargo compound is an RNA molecule selected from the group consisting of messenger ribonucleic acid (mRNA), microRNA (miRNA), small interfering RNA (siRNA), packaging RNA (pRNA), and transfer RNA (tRNA).

5. The method of claim 1, wherein the hydrophobic fluid stream comprises a solvent, a surfactant, and at least one of a long-chained cationic lipid and cholesterol, and optionally a fluorescently labeled lipid, a polymer-conjugated lipid, and hydrophobic cargo molecules comprising a therapeutic and/or diagnostic compound.

6. The method of claim 1, wherein the hydrophobic fluid stream comprises 25 wt % polysorbate-80, 40 wt % cholesterol, and 35 wt % dimethyldioctadecylammonium bromide (DDAB).

7. The method of claim 1, wherein the encapsulated therapeutics are non-ionic surfactant vehicles (NSVs), wherein the hydrophobic structural materials comprise a lipid composition with a non-ionic surfactant.

8. The method of claim 1, wherein the encapsulated therapeutics are gold nanoparticle-doped non-ionic surfactant vehicles (AuNSVs), wherein the hydrophobic structural materials comprise a lipid composition with a non-ionic surfactant, wherein the hydrophilic structural materials comprise gold nanoparticles (AuNPs).

9. A gold nanoparticle-doped non-ionic surfactant vehicle (AuNSV) made by the method of claim 8.

10. A gold nanoparticle-doped non-ionic surfactant vehicle (AuNSV) comprising a therapeutic agent and a gold nanoparticle (AuNP); wherein the AuNSV comprises an amorphous hydrophobic core containing cationic lipids, structural lipids, and a non-ionic surfactant; wherein the AuNSV has a substantially spherical geometry.

11. The AuNSV of claim 10, wherein the therapeutic agent is a biologic or a chemotherapeutic agent.

12. The AuNSV of claim 10, wherein the therapeutic agent is a messenger ribonucleic acid (mRNA), small interfering ribonucleic acid (siRNA), micro ribonucleic acid (miRNA), protein, a chemotherapeutics (doxorubicin, cisplatin), a small hydrophobic drug, or a CRISPR/Cas9 system.

13. The method of claim 10, wherein the AuNP is a 20 nm AuNP.

14. The AuNSV of claim 10, wherein the stability of the AuNSV is substantially maintained through freeze-thaw cycles, the stability of the AuNSV is substantially maintained through lyophilization, the AuNSV has reduced lysosomal co-localization in comparison to non-gold nanoparticle-doped NSV, the AuNSV has enhanced protein expression in comparison to non-gold nanoparticle-doped NSV, the AuNSV has enhanced vector uptake in comparison to non-gold nanoparticle-doped NSV, the AuNSV has activation of a shift in uptake pathway in comparison to non-gold nanoparticle-doped NSV, the AuNSV has inactivation of trafficking protein Rab7 in comparison to non-gold nanoparticle-doped NSV, the AuNSV has inactivation of uptake regulator PP2A in comparison to non-gold nanoparticle-doped NSV, and/or the AuNSV has improved cancer phenotypes in therapeutic delivery in vitro and in vivo.

15. The AuNSV of claim 10, wherein the AuNSV does not contain an ionizable lipid.

16. The AuNSV of claim 10, wherein the AuNSV is comprised in a solution of 5% sucrose.

17. The AuNSV of claim 10, wherein the AuNSV has a hydrodynamic diameter of about 150 nm as determined by DLS.

18. The AuNSV of claim 10, wherein the AuNSV do not have a lipid bilayer or multilamellar morphology.

19. A method of delivering a therapeutic agent to a patient in need thereof, the method comprising administering to the patient a therapeutically effective amount of the AuNSV of claim 10.

20. A method of treating a disease or disorder in a patient in need thereof, the method comprising administering to the patient a therapeutically effective amount of the AuNSV of claim 10.
